(19) United States
(12) Patent Application Publication
(10) Pub. No.: US 2026/0014176 A1
(43) Pub. Date: Jan. 15, 2026

(54) Structurally and Functionally Repaired Endothelial Glycocalyx

(71) Applicant: Northeastern University, Boston, MA (US)

(72) Inventors: Eno Essien Ebong, Milton, MA (US); Solomon A. Mensah, Malden, MA (US); Ronodeep Mitra, Boston, MA (US)

(21) Appl. No.: 19/267,491

(22) Filed: Jul. 11, 2025

Related U.S. Application Data
(60) Provisional application No. 63/670,827, filed on Jul. 12, 2024.

(51) Int. Cl. A61K 31/661; A61K 31/727; A61K 47/64; A61P 9/10

(52) U.S. Cl. CPC A61K 31/661; A61K 31/727; A61K 47/643; A61P 9/10

(57) ABSTRACT

Disclosed herein are compositions comprising a complex, wherein the complex comprises sphingosine-1-phosphate, a derivative thereof, or a pharmaceutically acceptable salt thereof, bound to a protein; and heparin, a derivative thereof, or a pharmaceutically acceptable salt thereof; related kits; and methods of making and using such compositions.

### RELATED APPLICATION

This application claims the benefit of U.S. Provisional Application No. 63/670,827, filed on Jul. 12, 2024. The entire teachings of the above application are incorporated herein by reference.

### GOVERNMENT SUPPORT

This invention was made with government support under Grant Number K01 HL125499 and under Grant Number R03 HL155244 awarded by the National Institutes of Health. The Government has certain rights in the invention.

### BACKGROUND

The inner lining of blood vessels is comprised of endothelial cells. On the surface of endothelial cells is a complex sugar mesh coat, called the endothelial glycocalyx. This structure shields the blood vessel wall from harmful molecules and cells that are flowing in the blood and which can infiltrate the wall and cause plaque formation. The endothelial glycocalyx is prone to degradation due to complex geometrical shapes of our blood vessels, including bifurcations, and due to chemical factors produced when the body is ill, such as reactive oxygen species. Damage to the endothelial glycocalyx represents a loss of blood vessel protection and has been attributed to the onset of blood vessel diseases, including atherosclerosis. Atherosclerosis is a precursor to many cardiovascular events, including heart attack and stroke. Even though there have been many improvements in detecting and treating such diseases in recent years, it is still one of the leading causes of death worldwide.

### SUMMARY

Many treatments currently available for vascular diseases (e.g., heart disease), such as statins or surgical intervention, target later stages of the diseases and there are a limited number of treatments that target early stages of the diseases. Furthermore, there are currently no pharmaceuticals that target the endothelial glycocalyx, even though it acts as a first line of defense against vascular disease.

Endothelial cell (EC) glycocalyx (GCX) shedding, e.g., from disturbed blood flow and chemical factors, leads to low-density lipoprotein infiltration, reduced nitric oxide synthesis, vascular dysfunction and atherosclerosis. In one embodiment, this disclosure relates to therapies combining sphingosine-1-phosphate (S1P) and heparin (heparan sulfate derivative). As described herein, in some embodiments, the disclosure relates to heparin/S1P co-treatment for repair of mechanically damaged EC GCX in disturbed flow (DF) regions and for restoration of anti-atherosclerotic mechanotransduction, e.g., to treat cardiovascular disease.

As disclosed herein, a parallel-plate flow chamber was used to simulate flow conditions in vitro and in a partial carotid ligation mouse model to mimic DF in vivo. Heparin and albumin-bound S1P were administered to assess their reparative effects on the endothelial GCX. Fluorescent staining, confocal microscopy, and ultrasound were used to evaluate endothelial cell function and endothelial-dependent vascular function. Barrier functionality was assessed via macrophage uptake. Heparin/S1P mechanism-of-action insights were gained through fluid dynamics simulations and staining of a GCX synthesis enzyme and S1P receptor. Statistical analyses validated the results.

The in vitro data showed that heparin/S1P therapy improves DF-conditioned ECs by restoring GCX and elevating vasodilator eNOS (endothelial-type nitric oxide synthase) expression. In vivo studies confirmed GCX degradation, vessel inflammation, hyperpermeability, and wall thickening in a partially ligated left carotid artery in a mouse model. Heparin/S1P treatment restored GCX thickness and coverage, reduced inflammation and hyperpermeability, and inhibited vessel wall thickening.

This work introduces a new approach to regenerating the EC GCX and restoring its function in ECs under DF conditions, offering a groundbreaking solution for preventing cardiovascular diseases like atherosclerosis.

In one embodiment, disclosed herein is a composition comprising a complex, wherein the complex comprises sphingosine-1-phosphate, a derivative thereof, or a pharmaceutically acceptable salt thereof, bound to a protein; and heparin, a derivative thereof, or a pharmaceutically acceptable salt thereof.

In one embodiment disclosed herein is a method of regenerating glycocalyx of a cell or inhibiting glycocalyx degradation of a cell, comprising contacting the cell with: a complex, wherein the complex comprises sphingosine-1-phosphate, a derivative thereof, or a pharmaceutically acceptable salt thereof, bound to a protein; and heparin, a derivative thereof, or a pharmaceutically acceptable salt thereof.

In another embodiment disclosed herein is a method of treating a disease or disorder, e.g., a vascular disease, in a subject in need thereof, comprising administering to the subject a composition comprising: a complex, wherein the complex comprises sphingosine-1-phosphate, a derivative thereof, or a pharmaceutically acceptable salt thereof, bound to a protein; and heparin, a derivative thereof, or a pharmaceutically acceptable salt thereof.

In another embodiment disclosed herein are a kits comprising a composition or complex of the present disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings are not necessarily to scale, emphasis instead being placed upon illustrating embodiments.

FIGS. 1A-1D: A dose-response study with heparin sodium was conducted to evaluate HS glycosaminoglycan (GAG) expression (green) on human coronary artery endothelial cells (HCAECs) after 12 hours under static conditions. The findings from this disclosure suggest that higher concentrations of heparin sodium can negatively impact the GCX structure, specifically the HS GAG component. Hence, a lower dosage of heparin (2 U/mL) was utilized throughout the in vitro studies based on these findings. Representative 63× images of HCAECs in all experimental groups are shown. Blue represents cell nuclei labeled with DAPI. FIG. 1A) No heparin treatment group. FIG. 1B) 2 U/mL heparin treated HCAECs. FIG. 1C) 100 U/mL heparin treated HCAECs. FIG. 1D) Normalized mean fluorescent intensity (MFI) data (comparing experimental conditions to no treatment) for the HS GAG component (green) component of the GCX, with n=2.

FIGS. 2A-2K: To establish a working immunocytochemistry protocol for active eNOS (p-eNOS; green) in HCAECs, a positive control, using L-arginine (L-Arg), which is a potent vasodilator and is the substrate for eNOS to generate nitric oxide (NO), was used. Furthermore, a negative control, using NG-Nitro-L-Arginine Methyl Ester (L-NAME), which is a non-selective NO synthase inhibitor, was also used. As suspected, L-Arg increased p-eNOS expression and L-NAME decreased p-eNOS expression, which suggests a working immunocytochemistry protocol for p-eNOS. Representative 63× images of HCAECs in all experimental groups are shown. Blue represents cell nuclei labeled with DAPI. The scale bar is 20 μm. FIG. 2A) p-eNOS expression in HCAECs with no L-Arg or L-NAME (baseline) at 30 minutes. FIG. 2B) p-eNOS expression in HCAECs with no L-Arg or L-NAME (baseline) at 1 hr. FIG. 2C) p-eNOS expression in ECs exposed to L-NAME (5 mM) for 30 minutes. FIG. 2D) p-eNOS expression in ECs exposed to L-NAME (5 mM) for 1 hr. FIG. 2E) p-eNOS expression in ECs exposed to L-Arg (5 mM) for 30 minutes. FIG. 2F) p-eNOS expression in ECs exposed to L-Arg (5 mM) for 1 hr. FIG. 2G) p-eNOS expression in ECs exposed to L-NAME (10 mM) for 30 minutes. FIG. 2H) p-eNOS expression in ECs exposed to L-NAME (10 mM) for 1 hr. FIG. 2I) p-eNOS expression in ECs exposed to L-Arg (10 mM) for 30 minutes. FIG. 2J) p-eNOS expression in ECs exposed to L-Arg (10 mM) for 1 hr. FIG. 2K) Graph shows the mean±SEM of percent area fraction of p-eNOS amongst different experimental groups (n=3). Statistical analysis was performed using two-way ANOVA. Significance is denoted by asterisks: *p<0.05.

FIG. 3: Dynamic light scattering (DLS) data that was utilized to determine effective diameter (nm) and polydispersity for fluid and particle flow simulations to determine residence time and concentrations in atheroprone DF region and atheroprotective UF region of in vitro parallel plate flow chamber.

FIGS. 4A-4H: Effect of co-treatment on WGA-labeled whole GCX expression in HCAECs after 12 hours of flow at 12 dynes/cm². Shown are 63× representative images of HCAECs in each experimental group. Fluorescent staining of wheat germ agglutinin (WGA; green), a lectin that stains multiple components of the GCX, was used to provide a comprehensive view to assess the percent area coverage of the whole structural GCX. WGA-labeled whole GCX expression increases in all conditions when co-treatment is administered with HCAECs. Blue represents cell nuclei labeled with DAPI. The scale bar is 20 μm. FIG. 4A) HCAECs in the no treatment group under static conditions. FIG. 4B) HCAECs in the no treatment group under atheroprone conditions. FIG. 4C) HCAECs in the no treatment group under atheroprotective conditions. FIG. 4D) HCAECs in the co-treatment group under static conditions. FIG. 4E) HCAECs in the co-treatment group under atheroprone conditions. FIG. 4F) HCAECs in the co-treatment group under atheroprotective conditions. FIG. 4G) Mean±SEM of percent area coverage of WGA staining normalized to the control (0 hr static condition) for no treatment group only. FIG. 4H) Mean±SEM of percent area coverage of WGA staining normalized to the control (0 hr static condition) for all cohorts (n=6). Statistical analysis was performed using a one or two-way ANOVA. Significance is denoted by asterisks: *p<0.05, **p<0.01, ***p<0.001, and ****p<0.0001. (See Table 3 for complete statistical analysis, and FIGS. 5D-5I and FIG. 5M for WGA-labeled whole GCX expression in HCAECs that were treated with albumin-bound S1P or heparin separately.)

FIGS. 5A-5N: Effect of different treatment groups on wheat germ agglutinin (WGA) expression in HCAECs after 12 hours of flow at 12 dynes/cm². Illumination of WGA (green), a lectin that stains multiple components of the GCX, was used to provide a comprehensive view and assessment of the percent area coverage of the whole structural GCX. Blue represents cell nuclei labeled with DAPI. The scale bar is 20 μm. These 63× confocal images with oil immersion and z-stacking are representative sections of cells in each group. FIG. 5A) 63× image with oil immersion and z-stacking of HCAECs in the no treatment group under static conditions. FIG. 5B) 63× image with oil immersion and z-stacking of HCAECs in the no treatment group in atheroprone conditions. FIG. 5C) 63× image with oil immersion and z-stacking of HCAECs in the no treatment group under atheroprotective conditions. FIG. 5D) 63× image with oil immersion and z-stacking of HCAECs in the heparin treated group under static conditions. FIG. 5E) 63× image with oil immersion and z-stacking of HCAECs in the heparin treated group in atheroprone conditions. FIG. 5F) 63× image with oil immersion and z-stacking of HCAECs in the heparin treated group in atheroprotective conditions. FIG. 5G) 63× image with oil immersion and z-stacking of HCAECs in the albumin-bound S1P treated group in static conditions. FIG. 5H) 63× image with oil immersion and z-stacking of HCAECs in the albumin-bound S1P treated group in atheroprone flow conditions. FIG. 5I) 63× image with oil immersion and z-stacking of HCAECs in the albumin-bound S1P treated group in atheroprotective flow conditions. FIG. 5J) 63× image with oil immersion and z-stacking of HCAECs in the co-treatment group in static conditions. FIG. 5K) 63× image with oil immersion and z-stacking of HCAECs in the co-treatment group in atheroprone flow conditions. FIG. 5L) 63× image with oil immersion and z-stacking of HCAECs in the co-treatment group in atheroprotective flow conditions. FIG. 5M) Mean±SEM of percent area coverage of WGA staining normalized to the control (0 hr static condition). FIG. 5N) Mean±SEM of percent area coverage of WGA staining normalized to the control (0 hr static condition) of only the no treatment group. The mean and error bars representing the SEM are included for each condition with n=6. Statistical analysis was performed using either one-way or two-way ANOVA. Significance is denoted by asterisks: *p<0.05, **p<0.01, ***p<0.001, and ****p<0.0001.

FIGS. 6A-6H: Effect of co-treatment on WGA-labeled whole GCX expression in murine carotid arteries. Shown are 40× representative images of carotid arteries from each cohort of mice. Fluorescent staining of wheat germ agglutinin (WGA; red), a lectin that stains multiple components of the GCX, was used to provide a comprehensive view of the whole structural GCX on the luminal side of the carotid arteries. The percent area coverage and the thickness of WGA increased following a single dose of the co-treatment administered via retro-orbital injection. Blue represents cell nuclei labeled with DAPI. Green represents elastin. The scale bar is 20 μm. FIG. 6A) Ligated left carotid artery (LCA) from the no vehicle group; arrow depicts degraded GCX. FIG. 6B) Control Right carotid artery (RCA) from the no vehicle group; arrow depicts intact GCX. FIG. 6C) Ligated LCA from the vehicle only group; arrow depicts degraded GCX. FIG. 6D) Control RCA in the vehicle only group; arrow depicts intact GCX. FIG. 6E) Ligated LCA from the co-treatment group; arrow depicts restored GCX. FIG. 6F) Control RCA from the co-treatment group; arrow depicts intact GCX. FIG. 6G) Graph shows the mean±SEM of percent area coverage of WGA staining normalized to the control (RCA of each mouse; n=6). FIG. 6H) Graph shows the mean±SEM of WGA transversal thickness normalized to the control (RCA of each mouse; n=6). Statistical analysis was performed using a one-way ANOVA. Significance is denoted by asterisks: *p<0.05, **p<0.01, ***p<0.001, and ****p<0.0001.

FIGS. 7A-7H: Effect of co-treatment on HS expression in HCAECs after 12 hours of flow at 12 dynes/cm². Shown are 63× representative images of HCAECs in each experimental group. Fluorescent staining of HS (green), a key GAG component of the GCX, increased in both the atheroprone and atheroprotective flow conditions with the co-treatment. Blue represents cell nuclei labeled with DAPI. The scale bar is 20 μm. FIG. 7A) HCAECs in the no treatment group under static conditions. FIG. 7B) HCAECs in the no treatment group under atheroprone conditions. FIG. 7C) HCAECs in the no treatment group under atheroprotective conditions. FIG. 7D) HCAECs in the co-treatment group under static conditions. 7E) HCAECs in the co-treatment group under atheroprone conditions. 7F) HCAECs in the co-treatment group under atheroprotective conditions. FIG. 7G) Mean±SEM of the MFI of HS staining normalized to the control (0 hr static condition) for no treatment group only. FIG. 7H) Mean±SEM of the MFI of HS staining normalized to the control (0 hr static condition) for all cohorts (n=6). Statistical analysis was performed using a one or two-way ANOVA. Significance is denoted by asterisks: *p<0.05, **p<0.01, ***p<0.001, and ****p<0.0001. (See Table 4 for complete statistical analysis, and FIG. 8D-8I and FIG. 8M for HS expression in HCAECs that were treated with albumin-bound S1P or heparin separately.)

FIGS. 8A-8N: Effects of co-treatment on HS expression in HCAECs after 12 hours of flow at 12 dynes/cm². Illumination of HS (green), a key component of the GCX, was used to assess the mean fluorescent intensity (MFI) of the GCX. The MFI of HS increases in both the atheroprone and atheroprotective conditions when treatment, particularly the co-treatment, is introduced to the flow system. Blue represents cell nuclei labeled with DAPI. The scale bar is 20 m. These 63× confocal images with oil immersion and z-stacking are representative sections of cells in each group. FIG. 8A) 63× image with oil immersion and z-stacking of HCAECs in the no treatment group under static conditions. FIG. 8B) 63× image with oil immersion and z-stacking of HCAECs in the no treatment group in atheroprone conditions. FIG. 8C) 63× image with oil immersion and z-stacking of HCAECs in the no treatment group under atheroprotective conditions. FIG. 8D) 63× image with oil immersion and z-stacking of HCAECs in the heparin treated group under static conditions. FIG. 8E) 63× image with oil immersion and z-stacking of HCAECs in the heparin treated group in atheroprone conditions. FIG. 8F) 63× image with oil immersion and z-stacking of HCAECs in the heparin treated group in atheroprotective conditions. FIG. 8G) 63× image with oil immersion and z-stacking of HCAECs in the albumin-bound S1P treated group in static conditions. FIG. 8H) 63× image with oil immersion and z-stacking of HCAECs in the albumin-bound S1P treated group in atheroprone flow conditions. FIG. 8I) 63× image with oil immersion and z-stacking of HCAECs in the albumin-bound S1P treated group in atheroprotective flow conditions. FIG. 8J) 63× image with oil immersion and z-stacking of HCAECs in the co-treatment group in static conditions. FIG. 8K) 63× image with oil immersion and z-stacking of HCAECs in the co-treatment group in atheroprone flow conditions. FIG. 8L) 63× image with oil immersion and z-stacking of HCAECs in the co-treatment group in atheroprotective flow conditions. FIG. 8M) Graph shows the mean±SEM of MFI of HS staining normalized to the control (0 hr static condition). FIG. 8N) Graph shows the mean±SEM of MFI of HS staining normalized to the control (0 hr static condition) of only no treatment group. The mean and error bars representing the SEM are included for each condition with n=6. Statistical analysis was performed using either one-way or two-way ANOVA. Significance is denoted by asterisks: *p<0.05, **p<0.01, ***p<0.001, and ****p<0.0001.

FIGS. 9A-9G: Effect of co-treatment on SDC1 (green), a GCX core protein, expression in HCAECs after 12 hours of flow at 12 dynes/cm². Shown are 63× representative images of HCAECs in each experimental group. SDC1 expression increases in all conditions when the co-treatment is introduced to HCAECs. Blue represents cell nuclei labeled with DAPI. The scale bar is 20 μm. FIG. 9A) HCAECs in the no treatment group under static conditions. FIG. 9B) HCAECs in the no treatment group under atheroprone conditions. FIG. 9C) HCAECs in the no treatment group under atheroprotective conditions. FIG. 9D) HCAECs in the co-treatment group under static conditions. FIG. 9E) HCAECs in the co-treatment group under atheroprone conditions. FIG. 9F) HCAECs in the co-treatment group under atheroprotective conditions. FIG. 9G) Graph shows the mean±SEM of percent area coverage of SDC1 staining normalized to the control (0 hr static condition; n=4). Statistical analysis was performed using a two-way ANOVA. Significance is denoted by asterisks: *p<0.05, **p<0.01, ***p<0.001, and ****p<0.0001.

FIGS. 10A-10H: Effect of co-treatment on SDC1 (red) expression in murine carotid arteries. Shown are representative 40× images of carotid arteries from each cohort. The percent area coverage and thickness of SDC1 increased in the co-treatment group following a single dose of the co-treatment administered via retro-orbital injection. Blue represents cell nuclei labeled with DAPI. Green represents elastin. The scale bar is 20 μm. FIG. 10A) Ligated LCA from the no vehicle group; SDC1 shedding is depicted by the arrow. FIG. 10B) Control RCA from the no vehicle group; intact SDC1 is depicted by the arrow. FIG. 10C) Ligated LCA from the vehicle only group; significant SDC1 shedding is depicted by the arrow. FIG. 10D) Control RCA from the vehicle only group; intact SDC1 is depicted by the arrow. FIG. 10E) Ligated LCA from the co-treatment group; newly expressed SDC1 is shown by the arrow. FIG. 10F) Control RCA from the co-treatment group; intact SDC1 is depicted by the arrow. FIG. 10G) Graph shows the mean±SEM of percent area coverage of SDC1 staining normalized to the control (RCA of each mouse; n=6). FIG. 10H) Graph shows the mean±SEM of SDC1 thickness normalized to the control (RCA of each mouse; n=6). Statistical analysis was performed using one-way ANOVA. Significance is denoted by asterisks: *p<0.05, **p<0.01, ***p<0.001, and ****p<0.0001.

FIGS. 11A-11G: Effect of co-treatment on GPC1 (green), a GCX core protein, expression in HCAECs after 12 hours of flow at 12 dynes/cm². Shown are representative 63× images of HCAECs in all experimental groups. The percent area coverage of GPC1 increased in atheroprone and atheroprotective conditions when co-treatment is introduced to the flow system. Blue represents cell nuclei labeled with DAPI. The scale bar is 20 μm. FIG. 11A) HCAECs in the no treatment group under static conditions. FIG. 11B) HCAECs in the no treatment group under atheroprone conditions. FIG. 11C) HCAECs in the no treatment group under atheroprotective conditions. FIG. 11D) HCAECs in the co-treatment group under static conditions. FIG. 11E) HCAECs in the co-treatment group under atheroprone conditions. FIG. 11F) HCAECs in the co-treatment group under atheroprotective conditions. FIG. 11G) Graph shows the mean±SEM of percent area coverage of GPC1 staining normalized to the control (0 hr static condition; n=4). Statistical analysis was performed using a two-way ANOVA. Significance is denoted by asterisks: *p<0.05, **p<0.01, ***p<0.001, and ****p<0.0001.

FIGS. 12A-12H: Effect of co-treatment on GPC1 (red) expression in murine carotid arteries. Shown are representative 40× images of carotid arteries of mice in all experimental cohorts. GPC1 percent area fraction and thickness increased in the co-treatment group following a single dose of the co-treatment administered via retro-orbital injection. Blue represents cell nuclei labeled with DAPI. Green represents elastin. The scale bar is 20 μm. FIG. 12A) Ligated LCA from the no vehicle group; GPC1 shedding is shown by the arrow. FIG. 12B) Control RCA from the no vehicle group; intact GPC1 is depicted by the arrow. FIG. 12C) Ligated LCA from the vehicle only group; GPC1 shedding is depicted by the arrow. FIG. 12D) Control RCA from the vehicle only group; intact GPC1 is depicted by the arrow. FIG. 12E) Ligated LCA from the co-treatment group; GPC1 synthesis is highlighted by the arrow. FIG. 12F) Control RCA from the co-treatment group; intact GPC1 is depicted by the arrow. FIG. 12G) Graph shows the mean±SEM of percent area fraction of GPC1 staining normalized to the control (RCA of each mouse; n=6). FIG. 12H) Graph shows the mean±SEM of GPC1 thickness normalized to the control (RCA of each mouse; n=6). Statistical analysis was performed using one-way ANOVA. Significance is denoted by asterisks: *p<0.05, **p<0.01, ***p<0.001, and ****p<0.0001.

FIGS. 13A-13H: In vitro cellular remodeling as indicated by EC morphology via cell alignment in relation to flow. Representative phase contrast images of HCAECs at 10× are shown for all cohorts. FIG. 13A) HCAECs exposed to static condition with no treatment. FIG. 13B) HCAECs exposed to atheroprone region with no treatment. FIG. 13C) HCAECs exposed to atheroprotective region with no treatment. FIG. 13D) HCAECs exposed to static condition with co-treatment. FIG. 13E) HCAECs exposed to atheroprone region with co-treatment. FIG. 13F) HCAECs exposed to atheroprotective region with co-treatment. All static and dynamic conditions (12 dynes/cm²) were for 12 hours. FIG. 13G) Cell alignment was analyzed using Object Orientation Parameter (OOP), which determines cell alignment in relation to flow. FIG. 13H) Cell alignment was analyzed using Median Pairwise Alignment (MPA), which determines cell alignment in relation to other ECs. Each data point on the graph represents the mean individual OOP or MPA from a single flow experiment, with cohort sizes ranging from n=16 to 20. Box and whisker plot in FIG. 13G for OOP shows the minimum, first quartile, median, third quartile, and maximum values for each condition. MPA in FIG. 13H shows the mean and error bars which represent the SEM. Statistical analysis was performed using a two-way ANOVA. Significance is denoted by asterisks: *p<0.05, **p<0.01, ***p<0.001, and ****p<0.0001.

FIGS. 14A-14G: Effect of co-treatment on activated eNOS (p-eNOS; green) expression in HCAECs after 12 hours of flow at 12 dynes/cm². Shown are representative 63× images of HCAECs from each experimental group. The percent area coverage of p-eNOS increased when the co-treatment is administered to the flow system. Blue represents cell nuclei labeled with DAPI. The scale bar is 20 μm. FIG. 14A) HCAECs in the no treatment group under static conditions. FIG. 14B) HCAECs in the no treatment group under atheroprone conditions. FIG. 14C) HCAECs in the no treatment group under atheroprotective conditions. FIG. 14D) HCAECs in the co-treatment group under static conditions. FIG. 14E) HCAECs in the co-treatment group under atheroprone conditions. FIG. 14F) HCAECs in the co-treatment group under atheroprotective conditions. FIG. 14G) Graph shows the mean±SEM of percent area coverage of p-eNOS staining normalized to the control (0 hr static condition; n=6). Statistical analysis was performed using a two-way ANOVA. Significance is denoted by asterisks: *p<0.05, **p<0.01, ***p<0.001, and ****p<0.0001. (See FIGS. 16D-16I and FIG. 16M for p-eNOS expression in HCAECs that were treated with either albumin-bound S1P or heparin separately.)

FIGS. 15A-15G: Effects of co-treatment on total eNOS (green) expression in HCAECs after 12 hours of flow at 12 dynes/cm². Shown are 63× representative images of total eNOS in HCAECs in different experimental groups. No changes were present in the percent area coverage of total eNOS with or without co-treatment. Blue represents cell nuclei labeled with DAPI. The scale bar is 20 μm. FIG. 15A) HCAECs in the no treatment group under static conditions. FIG. 15B) HCAECs in the no treatment group under atheroprone conditions. FIG. 15C) HCAECs in the no treatment group under atheroprotective conditions. FIG. 15D) HCAECs in the co-treatment group under static conditions. FIG. 15E) HCAECs in the co-treatment group under atheroprone conditions. FIG. 15F) HCAECs in the co-treatment group under atheroprotective conditions. FIG. 15G) Graph shows the mean±SEM of percent area coverage of total eNOS staining normalized to the control (0 hr static condition; n=6). Statistical analysis was performed using a two-way ANOVA. Significance is denoted by asterisks: *p<0.05, **p<0.01, ***p<0.001, and ****p<0.0001. (Note: FIGS. 23D-I and FIG. 23M contain results that show total eNOS expression in HCAECs that were treated with either albumin-bound S1P or heparin separately.)

FIGS. 16A-16N: Effect of different treatments on activated eNOS (p-eNOS) expression in HCAECs after 12 hours of flow at 12 dynes/cm². Illumination of p-eNOS (green), an enzyme that precedes the vasodilator eNO and contributes to vascular tone, was used to assess functional GCX. The percentage area fraction of p-eNOS increases in the atheroprone condition when various treatments, particularly co-treatment, is introduced to the flow system. Blue represents cell nuclei labeled with DAPI. The scale bar is 20 μm. These 63× confocal images with oil immersion and z-stacking are representative images of cells in each group. FIG. 16A) 63× image with oil immersion and z-stacking of HCAECs in the no treatment group under static conditions. FIG. 16B) 63× image with oil immersion and z-stacking of HCAECs in the no treatment group in atheroprone conditions. FIG. 16C) 63× image with oil immersion and z-stacking of HCAECs in the no treatment group under atheroprotective conditions. FIG. 16D) 63× image with oil immersion and z-stacking of HCAECs in the heparin treated group under static conditions. FIG. 16E) 63× image with oil immersion and z-stacking of HCAECs in the heparin treated group in atheroprone conditions. FIG. 16F) 63× image with oil immersion and z-stacking of HCAECs in the heparin treated group in atheroprotective conditions. FIG. 16G) 63× image with oil immersion and z-stacking of HCAECs in the albumin-bound S1P treated group in static conditions. FIG. 16H) 63× image with oil immersion and z-stacking of HCAECs in the albumin-bound S1P treated group in atheroprone flow conditions. FIG. 16I) 63× image with oil immersion and z-stacking of HCAECs in the albumin-bound S1P treated group in atheroprotective flow conditions. FIG. 16J) 63× image with oil immersion and z-stacking of HCAECs in the co-treatment group in static conditions. FIG. 16K) 63× image with oil immersion and z-stacking of HCAECs in the co-treatment group in atheroprone flow conditions. FIG. 16L) 63× image with oil immersion and z-stacking of HCAECs in the co-treatment group in atheroprotective flow conditions. FIG. 16M) Graph shows the mean±SEM of percent area fraction of p-eNOS normalized to the control (0 hr static condition) for all cohorts. FIG. 16N) Graph shows the mean±SEM of percent area fraction of p-eNOS normalized to the control (0 hr static condition) of only no treatment group. The mean and error bars representing the SEM are included for each condition with n=6. Statistical analysis was performed using either one-way or two-way ANOVA. Significance is denoted by asterisks: *p<0.05, **p<0.01, ***p<0.001, and ****p<0.0001.

FIGS. 17A-17D: FIG. 17A) Representative image of a carotid artery in B-mode in longitudinal axis and how different parameters were collected. FIG. 17B) Effect of co-treatment on vascular tone as indicated by vessel wall thickness (mm) via ultrasound images in vivo for experimental cohorts. FIG. 17C) Effect of co-treatment on vascular tone as indicated by systolic vessel diameter (mm) via ultrasound images in vivo for experimental cohorts. FIG. 17D) Effect of co-treatment on vascular tone as indicated by diastolic vessel diameter (mm) via ultrasound images in vivo for experimental cohorts. Co-treatment was able to provide vessel remodeling after a single retro-orbital IV injection in mice shown by reduction in vessel wall thickness; however, other parameters were not impacted. These data points were collected 5 days after ligation of LCA and mice were either given no vehicle, vehicle only, or co-treatment. Each data point on the graphs represents the mean individual for mouse in each cohort, where each cohort has an n=6. The mean and error bars representing the SEM. Statistical analysis was performed using a two-way ANOVA. Significance is denoted by asterisks: *p<0.05, **p<0.01, ***p<0.001, and ****p<0.0001.

FIGS. 18A-18G: Effect of co-treatment on CD68 expression in murine carotid arteries. Shown are 40× representative carotid arteries of mice in different experimental cohorts. Staining of CD68 (yellow), a protein expressed highly by circulating macrophages, was used to evaluate barrier permeability and macrophage uptake into the carotid arteries. CD68 uptake within vessel walls decreased in the co-treatment group following a single dose of the co-treatment administered via retro-orbital injection. Green represents elastin. The scale bar is 20 m. FIG. 18A) Ligated LCA from the no vehicle group; significant macrophage uptake within the vessel wall is depicted by the arrow. FIG. 18B) Control RCA from the no vehicle group; there is minimal macrophage uptake within the vessel wall. FIG. 18C) Ligated LCA arterial ring from the vehicle only group; significant macrophage uptake within the vessel wall is depicted by the arrow. FIG. 18D) Control RCA arterial ring from the vehicle only group; there is minimal macrophage uptake within the vessel wall. FIG. 18E) Ligated LCA from the co-treatment group; reduction in macrophage uptake within the vessel wall as shown by the arrow. FIG. 18F) Control RCA from the co-treatment group; macrophage uptake is undetectable within the vessel wall. FIG. 18G) Graph shows the mean±SEM of percent area fraction of CD68 staining within vessel walls of the LCA and RCA (n=6). Statistical analysis was performed using a two-way ANOVA. Significance is denoted by asterisks: *p<0.05, **p=0.01, ***p<0.001, and ****p<0.0001.

FIGS. 19A-19C: Fluid and particle dynamic simulations were conducted on parallel-plate flow chamber with co-treatment circulating the in vitro apparatus. FIG. 19A) Computational flow field around the step in the microfluidic device. Simulations show a clear region of recirculating flow downstream of the step. Black arrows illustrate the velocity vector field in the recirculating region. FIG. 19B) Computational particle analysis of co-treatment (red particles) circulating parallel-plate flow chamber. FIG. 19C) Particle transport simulations demonstrate that particles released within the recirculating flow region reside there for longer periods of time, causing a greater particle concentration relative to particles released outside of the recirculating region (snapshot from t=0.2 s following release). The black rectangle indicates the regions where particles were released. Note that a coarser mesh is used for visualization purposes only.

FIGS. 20A-20G: Effect of co-treatment on S1PR1 (green) expression in HCAECs after 12 hours of flow at 12 dynes/cm². Shown are representative 63× images of S1PR1 expression in HCAECs amongst different experimental groups. S1PR1, a G-protein coupled receptor found specifically on ECs, was used to determine its involvement in the therapeutic mechanism of action, and its expression increased in the atheroprotective and atheroprone regions when the co-treatment is introduced to the flow system, signifying activation. Blue represents cell nuclei labeled with DAPI. The scale bar is 20 μm. FIG. 20A) HCAECs in the no treatment group under static conditions. FIG. 20B) HCAECs in the no treatment group under atheroprone conditions. FIG. 20C) HCAECs in the no treatment group under atheroprotective conditions. FIG. 20D) HCAECs in the co-treatment group under static conditions. FIG. 20E) HCAECs in the co-treatment group under atheroprone conditions. FIG. 20F) HCAECs in the co-treatment group under atheroprotective conditions. FIG. 20G) Graph shows the mean±SEM of percent area coverage of S1PR1 staining normalized to the control (0 hr static condition; n=4). Statistical analysis was performed using a two-way ANOVA. Significance is denoted by asterisks: *p<0.05, **p<0.01, ***p<0.001, and ****p<0.0001. (See Table 5 for complete statistical analysis.)

FIGS. 21A-21G: Effect of the co-treatment on S1PR1 (red) expression in murine carotid arteries. Shown are 40× representative images of carotid arteries from mice from all experimental cohorts. S1PR1 (red), a G-protein coupled receptor found specifically on ECs, was used to explore a potential mechanism of action for the co-treatment. The percent area fraction of S1PR1 increases in the co-treatment group following a single dose of co-treatment administered via retro-orbital injection, signifying its activation. Green represents elastin. Blue represents cell nuclei. The scale bar is 20 μm. FIG. 21A) Ligated LCA from the no vehicle group; minimal S1PR1 expression as depicted by the arrow. FIG. 21B) Control RCA from the no vehicle group; arrow points to expressed S1PR1. FIG. 21C) Ligated LCA from the vehicle only group; minimal S1PR1 expression as depicted by the arrow. FIG. 21D) Control RCA from the vehicle only group; arrow points to expressed S1PR1. FIG. 21E) Ligated LCA from the co-treatment group; S1PR1 is restored and depicted by the arrow. FIG. 21F) Control RCA from the co-treatment group; arrow points to expressed S1PR1. FIG. 21G) Graph shows the mean±SEM of percent area fraction of S1PR1 staining normalized to the control (RCA of each mouse). The mean and error bars representing the SEM are included for each group, with n=3. Statistical analysis was performed using a two-way ANOVA. Significance is denoted by asterisks: *p<0.05, **p=0.01, ***p<0.001, and ****p<0.0001.

FIGS. 22A-22G: Effect of the co-treatment on EXTL3 (red) expression in murine carotid arteries. Shown are 40× representative images of carotid arteries from mice from all experimental cohorts. EXTL3 (red), which encodes the glycosyltransferases responsible for the biosynthesis of glycosaminoglycan heparan sulfate, was used to explore a potential mechanism of action for the co-treatment. The percent area fraction of EXTL3 increases in the co-treatment group following a single dose of co-treatment administered via retro-orbital injection, signifying its activation. Green represents elastin. Blue represents cell nuclei. The scale bar is 20 μm. FIG. 22A) Ligated LCA from the no vehicle group; low level of EXTL3 is depicted by the arrow. FIG. 22B) Control RCA from the no vehicle group; expressed EXTL3 is shown by the arrow. FIG. 22C) Ligated LCA from the vehicle only group; low level of EXTL3 is highlighted by the arrow. FIG. 22D) Control RCA from the vehicle only group; expressed EXTL3 is depicted by the arrow. FIG. 22E) Ligated LCA from the co-treatment group; expressed EXTL3 is depicted by the arrow. FIG. 22F) Control RCA from the co-treatment group; expressed EXTL3 is shown by the arrow. FIG. 22G) Graph shows the mean±SEM of percent area fraction of EXTL3 staining normalized to the control (RCA of each mouse). The mean and error bars representing the SEM are included for each group with n=6. Statistical analysis was performed using a two-way ANOVA. Significance is denoted by asterisks: *p<0.05, **p=0.01, ***p<0.001, and ****p<0.0001.

FIGS. 23A-23N: Effect of various treatments on total eNOS (active and inactive) expression in HCAECs after 12 hours of flow at 12 dynes/cm2. Illumination of total eNOS (green), an enzyme that precedes the vasodilator eNO and contributes to vascular tone, was used to assess functional GCX. No changes in percent area fraction was present in total eNOS expression with and without various treatments. Blue represents cell nuclei labeled with DAPI. The scale bar is 20 μm. These 63× confocal images with oil immersion and z-stacking are representative images of cells in each group. FIG. 23A) 63× image with oil immersion and z-stacking of HCAECs in the no treatment group under static conditions. FIG. 23B) 63× image with oil immersion and z-stacking of HCAECs in the no treatment group in atheroprone conditions. FIG. 23C) 63× image with oil immersion and z-stacking of HCAECs in the no treatment group under atheroprotective conditions. FIG. 23D) 63× image with oil immersion and z-stacking of HCAECs in the heparin treated group under static conditions. FIG. 23E) 63× image with oil immersion and z-stacking of HCAECs in the heparin treated group in atheroprone conditions. FIG. 23F) 63× image with oil immersion and z-stacking of HCAECs in the heparin treated group in atheroprotective conditions. FIG. 23G) 63× image with oil immersion and z-stacking of HCAECs in the albumin-bound S1P treated group in static conditions. FIG. 23H) 63× image with oil immersion and z-stacking of HCAECs in the albumin-bound S1P treated group in atheroprone flow conditions. FIG. 23I) 63× image with oil immersion and z-stacking of HCAECs in the albumin-bound S1P treated group in atheroprotective flow conditions. FIG. 23J) 63× image with oil immersion and z-stacking of HCAECs in the co-treatment group in static conditions. FIG. 23K) 63× image with oil immersion and z-stacking of HCAECs in the co-treatment group in atheroprone flow conditions. FIG. 23L) 63× image with oil immersion and z-stacking of HCAECs in the co-treatment group in atheroprotective flow conditions. FIG. 23M) Graph shows the mean±SEM of percent area fraction of total eNOS normalized to the control (0 hr static condition) for all cohorts. FIG. 23N) Graph shows the mean±SEM of percent area fraction of total eNOS normalized to the control (0 hr static condition) of only no treatment group. The mean and error bars representing the SEM are included for each condition with n=6. Statistical analysis was performed using either one-way or two-way ANOVA. Significance is denoted by asterisks: *p<0.05, **p<0.01, ***p<0.001, and ****p<0.0001.

### DETAILED DESCRIPTION

A description of example embodiments follows.

### Definitions

As used herein, the singular forms “a,” “an” and “the” include plural referents unless the context clearly dictates otherwise. Thus, for example, reference to “a” cell can include a plurality of cells. Further, the plurality can comprise more than one of the same cell or a plurality of different cells. Reference to an endothelial cell thus includes a plurality of endothelial cells, as make up the endothelium, for example. Accordingly, any of the methods described herein comprising endothelial cells can also be performed on endothelium.

As used herein, the term “subject” refers to an animal. Typically, the animal is a mammal. Examples of subjects include, for example, primates (e.g., humans), cows, sheep, goats, horses, dogs, cats, rabbits, rats, mice, fish, birds and the like. In some embodiments, the subject is a human.

As used herein, a subject (e.g., a human subject) is “in need of” a treatment if the subject has, or is at risk for developing, a disease or condition described herein (e.g., a vascular disease). Typically, a subject in need of treatment would benefit biologically, medically or in quality of life from such treatment A skilled medical professional (e.g. physician) can readily determine whether a subject has, or is at risk for developing, a disease or condition described herein.

As used herein, “treat”, “treating” or “treatment” means inhibiting or relieving a disease or disorder. For example, treatment can include a postponement of development of the symptoms associated with a disease or disorder, and/or a reduction in the severity of such symptoms that will, or are expected, to develop with said disease. The terms include ameliorating existing symptoms, preventing additional symptoms, and ameliorating or preventing the underlying causes of such symptoms. Thus, the terms denote that a beneficial result is being conferred on at least some of the mammals, e.g., human patients, being treated. Many medical treatments are effective for some, but not all, patients that undergo the treatment. “Treating,” as used herein, includes taking steps to deliver a therapy to a subject, such as a mammal, in need thereof (e.g., as by administering to a mammal one or more therapeutic agents). “Treating” includes preventing a disease or condition (e.g., a cardiovascular insult) from occurring in a subject, for example, when the subject is predisposed to the disease or condition, even if the subject has not yet been diagnosed with having the disease or condition; inhibiting the disease or condition (e.g., as by slowing or stopping its progression or causing regression of the disease or condition); and relieving the symptoms resulting from the disease or condition. In some embodiments, treatment includes improvement of a symptom in a cell, tissue or subject in comparison to the symptom in an untreated cell, tissue or subject (e.g., compared with a different subject, or with the same subject before and after treatment).

The terms “administer,” “administering,” or “administration,” in connection with any of the therapeutic agents described herein (e.g., heparin, a derivative, or a pharmaceutically acceptable salt thereof; sphingosine-1-phosphate, or a pharmaceutically acceptable salt thereof) refer to implanting, absorbing, ingesting, injecting, inhaling, or otherwise introducing a therapeutic agent(s), or a composition thereof, in, on or to a cell, tissue or subject (e.g., human).

An “effective amount” is an amount effective, at dosages and for periods of time necessary, to achieve a desired result (e.g., a desired therapeutic result). A “therapeutically effective amount” is an amount effective, at dosages and for periods of time necessary, to achieve a desired therapeutic result (e.g., treatment, healing, inhibition or amelioration of physiological response or condition, etc.). The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of a subject, mode of administration and the ability of a therapeutic, or combination of therapeutics, to elicit a desired response in a subject.

As used herein, the term “pharmaceutically acceptable salt” refers to those salts which are, within the scope of sound medical judgment, suitable for use in contact with the tissues of a subject without undue toxicity, irritation, allergic response and the like, and are commensurate with a reasonable benefit/risk ratio. Pharmaceutically acceptable salts are well known in the art. For example, S. M. Berge et al. describe pharmaceutically acceptable salts in detail in J. Pharmaceutical Sciences, 1977, 66, 1-19, the relevant teachings of which are incorporated herein by reference in their entirety. Pharmaceutically acceptable salts include salts derived from suitable inorganic and organic acids and inorganic and organic bases. Typically, a pharmaceutically acceptable salt of heparin will be a salt derived from a suitable inorganic or organic base, or a base addition salt.

As used herein, “a complex” is formed via non-covalent interactions between two or more moieties (such as a molecule and a protein), wherein the two or more moieties associate through intermolecular forces including, but not limited to, hydrogen bonding, electrostatic interactions, van der Waals forces, or hydrophobic interactions.

As used herein, “a derivative” refers to a compound that retains a core structural feature or functional group characteristic of a parent compound. For example, heparan sulfate is a derivative of heparin that differs in its degree and pattern of sulfation, but maintains a similar glycosaminoglycan backbone.

As used herein, “heparin” and “heparin sodium” are used interchangeably.

Atherosclerosis is a cardiovascular disease characterized by plaque development and rupture within arterial walls. Atherosclerosis in the context of coronary artery disease, which involves plaque formation in heart arteries, accounts for 75% of heart attacks and remains the leading cause of death both globally and in the United States [1]. Notably, 75% of these deaths occur in low-income and underserved regions [1,2]. Thus, developing novel, accessible, and cost-effective therapeutics is critical.

Endothelium, located between flowing blood and vessel tissue, facilitates vascular homeostasis [3-7], regulating selective permeability, inflammatory processes, and smooth muscle cell proliferation [3,6]. These functions rely on the endothelial glycocalyx (GCX), a negatively charged, hydrated polysaccharide plexus primarily located on the apical surface of endothelial cells (ECs) [8-10]. The GCX is comprised of glycoproteins, glycolipids, and proteoglycans like syndecan-1 (SDC1) and glypican-1 (GPC1), with glycosaminoglycans (GAGs) such as heparan sulfate (HS), hyaluronic acid, and chondroitin sulfate [3,11-13].

An intact GCX is vital for normal endothelial function, including regulation of vascular permeability and tone via mechanotransduction from undisturbed, uniform, laminar blood flow [4,5,11,14]. Disturbed blood flow patterns from complex vasculature structures can initiate GCX damage and shedding [3,5], resulting in disrupted biochemical signaling [15], increased vascular permeability and consequent inflammation [3,5,8], and reduced vascular tone [11,16,17]. For instance, GCX shedding promotes leukocyte and platelet adhesion [18-20], and GCX shedding upregulates intercellular adhesion molecule-1 (ICAM-1) and vascular cell adhesion molecule-1 (VCAM-1). This increases leukocyte adhesion and macrophage infiltration, triggering inflammation and macrophage infiltration into vessel walls, which ultimately leads to an inflammatory cascade response [21-23]. Furthermore, a direct consequence of EC GCX shedding is impaired vascular tone, where endothelial nitric oxide synthase (eNOS), a precursor to vasodilator nitric oxide (NO), decreases in activity, which reduces NO bioavailability and causes vasoconstriction [10,24,25]. This impairment is linked to diabetes and hypertension, causing insulin resistance and high blood pressure, respectively [26,27]. Additionally, atherosclerosis onset is often attributed to non-uniform blood flow patterns [3,11,12], which cause GCX degradation and subsequent endothelial dysfunction [17]. Thus, targeting the GCX is a crucial first step for preventing and treating cardiovascular disease.

It has been shown that a combined treatment of exogenous HS and sphingosine-1-phosphate (SIP) repaired GCX structure and restored vasculoprotective EC-to-EC communication function in rat fat pad ECs that had experienced enzymatically induced damage to their GCX and intercellular communication interruption [28]. HS is the predominant GAG component of the GCX on ECs (>70%) [29]. S1P is a bioactive lipid mediator that stabilizes the vascular system and GCX by enhancing intercellular junction strength via zonula occluden-1 (ZO-1) and connexin, thereby regulating transendothelial permeability [28,30-36].

In this disclosure, the ability of derivatives of HS and S1P to effectively repair damaged GCX structurally and functionally in the initial stages of atherosclerosis in both in vitro and in vivo conditions was tested. The co-treatment was modified with a more affordable and commercially available derivative to exogenous HS, heparin sodium. Heparin is a widely used anticoagulant that protects and synthesizes endothelial GCX [37]. Specifically, heparin has been indicated to prevent and treat blood clots in patients at risk for thrombotic events and during certain medical procedures [38,39]. It has also been shown to inhibit the shedding of endothelial GCX components into the circulation in disease-free rats [40]. Albumin, a naturally occurring chaperone, was utilized to stabilize S1P in vitro and in vivo [41,42]. The albumin chaperone bound to S1P improves S1P stability and bioactivity [33].

This new and modified co-treatment was utilized in more relevant in vitro and in vivo endothelial dysfunction models. One model was achieved by using human coronary artery endothelial cells (HCAECs) cultured in vitro using a parallel-plate flow chamber in which disturbed flow (DF) was generated adjacent to undisturbed, uniform, laminar blood flow [11,16]. An in vivo model implemented a partial ligation surgery of the left carotid artery (LCA) in a murine model [3,5,43]. Both the in vitro and in vivo models contained neighboring atherosclerotic-prone DF regions and atheroprotective uniform flow (UF) regions. Upon administration of the co-treatment of albumin-bound S1P (in vitro and in vivo: 1.5 uM) and heparin (in vitro: 2 U/mL; in vivo: 500 U/kg) it was determined whether, in early atherosclerosis, GCX damage could be reversed and proper endothelial function (eNOS expression, minimized vascular remodeling, and blocked inflammatory cell infiltration) could be restored. To do this, endothelial functions, including barrier function and vascular tone, were assessed. The co-treatment's potential mechanism(s) of action, including the possibility of a mechanical mechanism, was also investigated. Finally, the possibility of a biochemical mechanism, either involving targeting of an S1P receptor, or involving chemical synthesis via upregulation of exostosin-like glycosyltransferase-3 (EXTL3) that synthesizes HS in the Golgi, was probed.

The disclosure described herein provides evidence of the potential of HS- and S1P-derived co-treatment to effectively reverse GCX damage and restore proper endothelial function. Moreover, underlying mechanisms governing its therapeutic effect are elucidated. These findings are pivotal for advancing the translation of this treatment into clinical practice, offering a promising avenue for improving cardiovascular disease management and ultimately enhancing patient outcomes.

Embodiments of the present disclosure relate to a composition comprising:
a complex, wherein the complex comprises sphingosine-1-phosphate (S1P), a derivative thereof, or a pharmaceutically acceptable salt thereof, bound to a protein; and
heparin, a derivative thereof, or a pharmaceutically acceptable salt thereof.

In some embodiments, a pharmaceutically acceptable salt of heparin is sodium heparin, calcium heparin, potassium heparin, or magnesium heparin. In some embodiments, the pharmaceutically acceptable salt of heparin is heparin sodium (e.g., heparin sodium from porcine intestinal mucosa). Heparin is a polymer classified as a mucopolysaccharide or a glycosaminoglycan, and is biosynthesized and stored in mast cells of various mammalian tissues, including liver, lung, and mucosa [93].

S1P is a bioactive sphingolipid metabolite that enhances cellular responses including cell growth, survival, migration, decrease in endothelial layer permeability, and preventing syndecan-1 shedding. It works by binding to a specific subfamily of G-protein-coupled receptors [94]. S1P may be bound to a chaperone (e.g., a protein such as albumin) to increase circulation time. In some embodiments, the protein bound to sphingosine-1-phosphate, a derivative thereof, or a pharmaceutically acceptable salt thereof is naturally-occurring or synthetic. For example, S1P-binding proteins may include, but are not limited to, apolipoprotein A-I, apolipoprotein A-IV, high-density lipoprotein (HDL)-associated proteins, low-density lipoprotein (LDL)-associated proteins, gelsolin, al-acid glycoprotein, ceruloplasmin, transferrin, and clusterin. In some embodiments, the protein bound to sphingosine-1-phosphate, a derivative thereof, or a pharmaceutically acceptable salt thereof is apolipoprotein M or albumin. In some embodiments, the protein is a fusion protein (e.g., apolipoprotein M fused to a fragment crystallizable (Fc) region of an antibody). The complex of apolipoprotein M fused to a Fc region and S1P has been shown to restore GCX and nitric oxide synthase. In some embodiments, the protein is apolipoprotein M. In other embodiments, the protein is albumin.

Sphingosine-1-phosphate (S1P), a derivative thereof, or a pharmaceutically acceptable salt thereof, may be bound to a protein by hydrophobic or hydrophilic interactions.

In some embodiments, a derivative of heparin is heparan sulfate. In some embodiments, the composition comprises: the complex; and heparin or a pharmaceutically acceptable salt of heparin (e.g., heparin potassium, heparin sodium, etc.). In some embodiments, the composition comprises: a complex (e.g., a protein-S1P complex); and heparin sodium.

In some embodiments, a complex has a concentration of about 0.1 nM to about 10 mM (e.g., about 0.1 nM to about 1 mM, about 1 nM to about 1 mM, about 10 nM to about 1 mM, about 10 nM to about 0.1 mM, about 10 nM to about 10 μM, about 0.1 μM to about 10 μM, about 0.1 μM to about 5 μM, about 1 μM to about 10 μM, about 0.1 μM to about 1 μM, about 1 M to about 5 μM, etc.). In some embodiments, the complex has a concentration of about 0.1 M to about 10 μM. In some embodiments, the complex has a concentration of about 0.1 μM to about 5 μM. In some embodiments, the complex has a concentration of about 1.5 μM.

In some embodiments, heparin, a derivative thereof, or a pharmaceutically acceptable salt thereof, has a concentration of about 0.01 U/mL to about 20 U/mL (e.g., about 0.01 U/mL to about 15 U/mL, about 0.01 U/mL to about 10 U/mL, about 0.05 U/mL to about 10 U/mL, about 0.1 U/mL to about 10 U/mL, about 0.1 U/mL to about 5 U/mL, about 0.5 U/mL to about 5 U/mL, about 1 U/mL to about 5 U/mL, etc.). In some embodiments, heparin, a derivative thereof, or a pharmaceutically acceptable salt thereof, has a concentration of about 0.1 U/mL to about 10 U/mL. In some embodiments, heparin, a derivative thereof, or a pharmaceutically acceptable salt thereof, has a concentration of about 1 U/mL to about 5 U/mL. In some embodiments, heparin, a derivative thereof, or a pharmaceutically acceptable salt thereof, has a concentration of about 2 U/mL.

In some embodiments, heparin, a derivative thereof, or a pharmaceutically acceptable salt thereof, is present in an amount of about 1 U/kg to about 10000 U/kg (e.g., about 1 U/kg to about 9000 U/kg, about 1 U/kg to about 8000 U/kg, about 1 U/kg to about 7000 U/kg, about 1 U/kg to about 6000 U/kg, about 1 U/kg to about 5000 U/kg, about 1 U/kg to about 4000 U/kg, about 1 U/kg to about 3000 U/kg, about 1 U/kg to about 2000 U/kg, about 1 U/kg to about 1000 U/kg, etc.) based on a subject's weight. In some embodiments, heparin, a derivative thereof, or a pharmaceutically acceptable salt thereof, is present in an amount of about 100 U/kg to about 1000 U/kg based on a subject's weight. In some embodiments, heparin, a derivative thereof, or a pharmaceutically acceptable salt thereof, is present in an amount of about 400 U/kg to about 600 U/kg based on a subject's weight.

The appropriate dosage for increased efficacy of a composition may be dependent on blood volume and the seriousness of the pathology. In some embodiments, a composition comprises about 1.5 μM of a protein-bound SIP complex and about 2 U/mL of heparin sodium. In some embodiments, a composition comprises about 1.5 μM of a protein-bound SIP complex and about 500 U/kg of heparin or a pharmaceutically acceptable salt thereof based on a subject's weight.

In some embodiments disclosed herein is a composition comprising heparin or heparin sodium and glycocalyx protector sphingosine-1-phosphate (SIP). In some embodiments, SIP is chaperoned by albumin (albumin-SIP). Albumin-S1P provides existing pre-clinical and clinical use, the ability to ensure in vivo stability of the formulation, and cost advantage. Binding S1P to albumin improved its stability in solution and allowed a ˜10-fold reduction in effective concentration compared to unbound S1P (previously used at 10 μM).

Embodiments of the present disclosure also relate to conjugates comprising: sphingosine-1-phosphate, a derivative thereof, or a pharmaceutically acceptable salt thereof; and heparin, a derivative thereof, or a pharmaceutically acceptable salt thereof.

In some embodiments, heparin sodium is conjugated with a protein (e.g., albumin) to enable co-delivery.

In some embodiments, a conjugate comprises sphingosine-1-phosphate, a derivative thereof, or a pharmaceutically acceptable salt thereof, conjugated to heparin, a derivative thereof, or a pharmaceutically acceptable salt thereof, through a linker. In some embodiments, the linker is a triazole linker. Click chemistry may be employed to form a conjugate of the present disclosure (e.g., via azide/alkyne modifications to covalently link azide-modified heparin sodium with alkyne-modified S1P).

In some embodiments, the composition comprises sphingosine-1-phosphate, a derivative thereof, or a pharmaceutically acceptable salt thereof, bound to a protein; and heparin, a derivative thereof, or a pharmaceutically acceptable salt thereof.

In some embodiments, the conjugate is

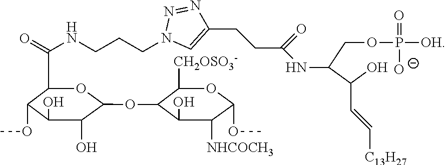

In some embodiments, a conjugate is a micelle. In some embodiments, micelles are formed with protein-bound (e.g., albumin-bound) sphingosine-1-phosphate (S1P) and chemical groups (e.g., hydrophilic groups) are introduced to the protein-S1P complex. In some embodiments, the chemical groups are hydrophilic, which promote electrostatic interactions with negatively charged salts of heparin such as heparin sodium.

In some embodiments, a composition comprises a conjugate of the present disclosure.

### Methods

In some embodiments, compositions, complexes, compounds, and conjugates of the present disclosure can be used for the repair of degraded endothelial glycocalyx and/or to restore proper endothelial cell function. The compositions, complexes, compounds, and conjugates may delay the onset of atherosclerosis and other (e.g., related) cardiovascular disease conditions. Compositions, complexes, compounds, and conjugates of the present disclosure may be delivered through various routes (orally, intravenously, etc.), and applied to carriers including circulating nanoparticle or immobilized biomaterial drug carriers, and to be used as a coating on stents and catheters.

The technology described herein targets the front-line issue in cardiovascular disease: glycocalyx degradation and endothelial cell dysfunction. Currently, therapeutic approaches target symptoms and late stage disease. Statins are used in a non-targeted manner and are associated with undesirable off target effects. Surgery is invasive and can lead to repeated incidence of vascular disease.

In some embodiments, albumin-bound S1P and heparin co-treatment enhances endothelial glycocalyx integrity, assisting in glycocalyx regeneration. In some embodiments, reduces vascular wall hyperpermeability and improves vessel tone, assisting in endothelial function restoration. In some embodiments, co-treatment is potent in atheroprone regions due to higher concentration and residence times. In some embodiments, co-treatment increases S1P receptor 1 levels and upregulates heparan sulfate synthesis enzyme EXTL3.

In some embodiments, the protein-bound S1P and heparin are administered separately, (either sequentially or simultaneously) e.g., in separate compositions and/or through separate routes of administration. In some embodiments they are administered in the same composition.

In some embodiments, the methods, compositions, complexes, compounds, and conjugates of the present disclosure possess the ability to replace lost heparan sulfate and other components from the endothelial glycocalyx. In some embodiments, they possess the ability to restore glycocalyx-dependent barrier function; glycocalyx-mediated endothelial cell mechanotransduction through production of nitric oxide; and/or other key endothelial cell functions. In some embodiments, they possess the ability to recover glycocalyx-dependent cell-to-cell communication.

In some embodiments, the components are biologically derived, so minimal host response is expected. Sulodexide contains heparin and dermatan sulfate and is used as an anticoagulant but the latter component is not naturally present in blood vessel endothelial cells. GlycoFix-2 uses exogenous heparin (derivative of heparin sodium) alone and in combination with no foreign polysaccharide. GlycoFix-2 simultaneously replaces endothelial glycocalyx components and achieves restoration of complicated cellular functions. Heparin and chaperone bound S1P restores barrier function and production of nitric oxide, and repairs cell-to-cell communication function, which are difficult to recover and are important parts of endothelial function. Nutraceuticals arterosil and endocalyx have insufficient levels of clinical data and no peer-reviewed publications. Hence, they are not currently FDA approved, which can have limited capability of reaching and impacting patients.

In some embodiments, the compositions, complexes, compounds, and conjugates of the present disclosure can be administered, e.g., orally or intravenously administered, to subjects (e.g., patients) to prevent glycocalyx loss due to increased inflammation by disease or during surgeries. In some embodiments, they can be administered, e.g., orally administered, to subjects (e.g., patients) suffering from excessive shedding of glycocalyx due to increased inflammation by the presence of a disease or during surgeries. In some embodiments, they are used as a device (e.g., stent) coating to aid in vascular endothelial cell recovery, e.g., after device deployment. In some embodiments, they are incorporated with circulating nanoparticle or immobilized carriers, e.g., biomaterial drug carriers.

In some embodiments disclosed herein are methods of regenerating glycocalyx (e.g., endothelial glycocalyx) of a cell, comprising contacting the cell with (e.g., contacting the cell with an effective amount of):
a complex, wherein the complex comprises sphingosine-1-phosphate, a derivative thereof, or a pharmaceutically acceptable salt thereof, bound to a protein; and ii) heparin, a derivative thereof, or a pharmaceutically acceptable salt thereof,
or a conjugate of the present disclosure.

Glycocalyx regeneration includes increasing (e.g., restoring to baseline or physiologically normal or beneficial levels) the thickness of the glycocalyx, increasing (e.g., restoring to baseline or physiologically normal or beneficial levels) the coverage of the glycocalyx on a cell, increasing (e.g., restoring to baseline or physiologically normal or beneficial levels) expression of a connexin by a cell, increasing (e.g., restoring to baseline or physiologically normal or beneficial levels) stability of the plasma membrane of a cell, improving (e.g., restoring to baseline or physiologically normal or beneficial levels) gap junction function of a cell, and increasing (e.g., restoring to baseline or physiologically normal or beneficial levels) intercellular communication between two or more cells.

In some embodiments disclosed herein are methods of increasing expression of a connexin by a cell, comprising contacting the cell with:
a complex, wherein the complex comprises sphingosine-1-phosphate, a derivative thereof, or a pharmaceutically acceptable salt thereof, bound to a protein; and ii) heparin, a derivative thereof, or a pharmaceutically acceptable salt thereof,
or a conjugate of the present disclosure.

In some embodiments disclosed herein are methods of increasing stability of the plasma membrane of a cell, comprising contacting the cell with:
a complex, wherein the complex comprises sphingosine-1-phosphate, a derivative thereof, or a pharmaceutically acceptable salt thereof, bound to a protein; and ii) heparin, a derivative thereof, or a pharmaceutically acceptable salt thereof,
or a conjugate of the present disclosure.

In some embodiments disclosed herein are methods of improving gap junction function of a cell, comprising contacting the cell with:
a complex, wherein the complex comprises sphingosine-1-phosphate, a derivative thereof, or a pharmaceutically acceptable salt thereof, bound to a protein; and ii) heparin, a derivative thereof, or a pharmaceutically acceptable salt thereof,
or a conjugate of the present disclosure.

In some embodiments disclosed herein are methods of increasing intercellular communication between two or more cells, comprising contacting at least one of the two or more cells with:
a complex, wherein the complex comprises sphingosine-1-phosphate, a derivative thereof, or a pharmaceutically acceptable salt thereof, bound to a protein; and ii) heparin, a derivative thereof, or a pharmaceutically acceptable salt thereof,
or a conjugate of the present disclosure.

In some embodiments disclosed herein are methods of inhibiting glycocalyx degradation of a cell, comprising contacting the cell with:
a complex, wherein the complex comprises sphingosine-1-phosphate, a derivative thereof, or a pharmaceutically acceptable salt thereof, bound to a protein; and ii) heparin, a derivative thereof, or a pharmaceutically acceptable salt thereof,
or a conjugate of the present disclosure.

Glycocalyx degradation includes decreasing (e.g., to below baseline or physiologically normal or beneficial levels) the thickness of the glycocalyx, decreasing (e.g., to below baseline or physiologically normal or beneficial levels) the coverage of the glycocalyx on a cell, decreasing (e.g., to below baseline or physiologically normal or beneficial levels) expression of a connexin by a cell, decreasing (e.g., to below baseline or physiologically normal or beneficial levels) stability of the plasma membrane of a cell, decreasing (e.g., to below baseline or physiologically normal or beneficial levels) gap junction function of a cell, and decreasing (e.g., to below baseline or physiologically normal or beneficial levels) intercellular communication between two or more cells. Inhibition of glycocalyx degradation includes slowing (e.g., the rate of glycocalyx degradation), halting and reversing (e.g., regenerating, for example, to baseline or physiologically normal or beneficial levels) glycocalyx degradation.

In some embodiments disclosed herein are methods of treating a vascular disease in a subject in need thereof, comprising administering to the subject (e.g., a therapeutically effective amount of) a composition comprising: a complex, wherein the complex comprises sphingosine-1-phosphate, a derivative thereof, or a pharmaceutically acceptable salt thereof, bound to a protein; and heparin, a derivative thereof, or a pharmaceutically acceptable salt thereof. In some embodiments, the subject is a human subject.

In some embodiments disclosed herein are methods of regenerating glycocalyx of a cell or inhibiting glycocalyx degradation of a cell, comprising contacting the cell with: a complex, wherein the complex comprises sphingosine-1-phosphate, a derivative thereof, or a pharmaceutically acceptable salt thereof, bound to a protein; and heparin, a derivative thereof, or a pharmaceutically acceptable salt thereof.

In some embodiments, the cell is a mammalian cell. In some embodiments, the cell is an endothelial cell, epithelial cell, red blood cell, cancer cell, or immune cell (e.g., lymphocyte, monocyte). In some embodiments, the cell is an epithelial cell. In some embodiments, the cell is an endothelial cell.

In some embodiments disclosed herein are methods of treating a vascular disease in a subject in need thereof, comprising administering to the subject:
a complex, wherein the complex comprises sphingosine-1-phosphate, a derivative thereof, or a pharmaceutically acceptable salt thereof, bound to a protein; and ii) heparin, a derivative thereof, or a pharmaceutically acceptable salt thereof,
or a conjugate of the present disclosure.

In some embodiments, the vascular disease is atherosclerosis, a blood clot, a stroke, peripheral artery disease, an aneurysm, a pulmonary embolism, carotid artery disease, arteriovenous malformation, critical limb ischemia, deep vein thrombosis, chronic venous insufficiency, a varicose vein, coronary artery disease, Raynaud's disease, sepsis, von Willebrand disease or vasculitis. In some embodiments, the vascular disease is atherosclerosis.

In some embodiments, the vascular disease is an early stage of vascular disease.

In some embodiments, the heparin, the derivative thereof, or the pharmaceutically acceptable salt thereof, is derived from an exogenous source. In some embodiments, the exogenous source is porcine intestinal mucosa. In some embodiments, the derivative of heparin is heparan sulfate. In some embodiments, the pharmaceutically acceptable salt of heparin is heparin sodium.

### Preparation and Administration

Compositions described herein can be prepared by any suitable method known in the art of pharmacology. In general, such preparatory methods include bringing a complex (e.g., protein-bound S1P), a conjugate, and/or one or more compounds described herein (e.g., heparin, a derivative thereof, or a pharmaceutically acceptable salt thereof) into association with a carrier or excipient, and/or one or more other accessory ingredients, and then, if necessary and/or desirable, shaping, and/or packaging the product into a desired single- or multi-dose unit. In some embodiments, the compositions are adapted for oral administration.

In some embodiments disclosed herein are methods of forming a composition, the methods comprising: combining a complex with heparin, a derivative thereof, or a pharmaceutically acceptable salt thereof, thereby forming the composition, wherein the complex comprises sphingosine-1-phosphate, a derivative thereof, or a pharmaceutically acceptable salt thereof, bound to a protein (e.g., albumin), and wherein the composition comprises the complex and heparin, the derivative thereof, or the pharmaceutically acceptable salt thereof.

Compositions (e.g., pharmaceutical compositions) can be prepared, packaged, and/or sold in bulk, as a single unit dose, and/or as a plurality of single unit doses. A “unit dose” is a discrete amount of the composition comprising a predetermined amount of the therapeutic agent (i.e., a complex (e.g., protein-bound S1P), a conjugate, and/or one or more compounds described herein (e.g., heparin, a derivative thereof, or a pharmaceutically acceptable salt thereof)). In some embodiments, the therapeutic agent comprises protein-bound S1P (e.g., albumin-bound S1P) and heparin sodium. The amount of the therapeutic agent is generally equal to the dosage of the therapeutic agent which would be administered to a subject and/or a convenient fraction of such a dosage, such as one-half or one-third of such a dosage.

Relative amounts of the therapeutic agent (e.g., a complex, heparin, a derivative thereof, or a pharmaceutically acceptable salt thereof, a conjugate), the pharmaceutically acceptable carrier or excipient, and/or any additional ingredients in a pharmaceutical composition described herein will vary, depending, for example, upon the identity, size, and/or condition of the subject treated and upon the route by which the composition is to be administered. The composition may comprise between 0.1% and 100% (w/w) therapeutic agent.

Pharmaceutically acceptable excipients used in the manufacture of compositions disclosed herein include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Compositions provided herein can be orally administered in any orally acceptable dosage form including, but not limited to, capsules, tablets, pills, powders, granules, aqueous suspensions, dispersions and solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions and/or emulsions are required for oral use, the therapeutic agent can be suspended or dissolved in an oily phase and combined with emulsifying and/or suspending agents. If desired, certain sweetening, flavoring or coloring agents may also be added.

Examples of excipients or carriers include sodium citrate or dicalcium phosphate and/or (a) fillers or extenders such as starches, lactose, sucrose, glucose, mannitol, and silicic acid, (b) binders, such as carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium salts, (g) wetting agents, such as acetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Liquid dosage forms for oral administration include pharmaceutically acceptable emulsions, microemulsions, solutions, suspensions, syrups, and elixirs. In addition to the therapeutic agent(s), the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, or mixtures thereof. Besides inert diluents, the oral compositions can also include adjuvants, such as wetting agents, emulsifying and suspending agents, sweetening, flavoring, coloring, perfuming, and preservative agents.

Compositions suitable for buccal or sublingual administration include tablets, lozenges and pastilles, wherein the therapeutic agent(s) is formulated with a carrier such as sugar and acacia, tragacanth, or gelatin and glycerin.

Solid compositions of a similar type may also be employed as fillers in soft and hard-filled gelatin capsules using excipients such as lactose or milk sugar, as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the therapeutic agent(s) only, or preferentially, in a certain part of the intestinal tract, optionally, in a delayed manner. Examples of embedding compositions that can be used include polymeric substances and waxes.

A composition disclosed herein can also be in micro-encapsulated form. In such solid dosage forms, the therapeutic agent(s) can be admixed with at least one inert diluent such as sucrose, lactose or starch. Such dosage forms can also comprise, as is normal practice, additional substances other than inert diluents, e.g., tableting lubricants and other tableting aids such a magnesium stearate and microcrystalline cellulose.

Compositions for oral administration may be designed to protect the therapeutic agent(s) against degradation as it passes through the alimentary tract, for example, by an outer coating of the formulation on a tablet or capsule.

In another embodiment, a composition can be provided in an extended (or “delayed” or “sustained”) release composition. This delayed-release composition comprises the therapeutic agent(s) in combination with a delayed-release component. Such a composition allows targeted release of a provided agent into the lower gastrointestinal tract, for example, into the small intestine, the large intestine, the colon and/or the rectum. In certain embodiments, a delayed-release composition further comprises an enteric or pH-dependent coating, such as cellulose acetate phthalates and other phthalates polyvinyl acetate phthalate, methacrylates (Eudragits)). Alternatively, the delayed-release composition provides controlled release to the small intestine and/or colon by the provision of pH-sensitive methacrylate coatings, pH-sensitive polymeric microspheres, or polymers which undergo degradation by hydrolysis. The delayed-release composition can be formulated with hydrophobic or gelling excipients or coatings, Colonic delivery can further be provided by coatings which are digested by bacterial enzymes such as amylose or pectin, by pH-dependent polymers, by hydrogel plugs swelling with time (Pulsincap), by time-dependent hydrogel coatings and/or by acrylic acid linked to azoaromatic bonds coatings.

Compositions described herein can also be administered subcutaneously, intraperitoneally or intra-venously. Compositions described herein for intravenous, subcutaneous, or intraperitoneal injection may contain an isotonic vehicle such as sodium chloride injection, Ringer's injection, dextrose injection, dextrose and sodium chloride injection, lactated Ringer's injection, or other vehicles known in the art.

Compositions described herein can also be administered in the form of suppositories for rectal administration. These can be prepared by mixing the therapeutic agent(s) with a suitable non-irritating excipient that is solid at room temperature but liquid at rectal temperature and, therefore, will melt in the rectum to release the drug. Such materials include cocoa butter, beeswax and polyethylene glycols.

Compositions described herein can also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Transdermal patches can also be used for topical applications.

For other topical applications, the compositions can be formulated in a suitable ointment containing the therapeutic agent(s) suspended or dissolved in one or more carriers, Carriers for topical administration include, but are not limited to, mineral oil, liquid petrolatum, white petrolatum, propylene glycol, polyoxyethylene, polyoxypropylene compound, emulsifying wax and water and penetration enhancers. Alternatively, compositions can be formulated in a suitable lotion or cream containing the therapeutic agent(s) suspended or dissolved in one or more pharmaceutically acceptable carriers. Alternatively, the composition can be formulated with a suitable lotion or cream containing the therapeutic agent(s) suspended or dissolved in a carrier with suitable emulsifying agents. In some embodiments, suitable carriers include, but are not limited to, mineral oil, sorbitan monostearate, polysorbate 60, cetyl esters wax, cetearyl alcohol, 2-octyldodecanol, benzyl alcohol and water. In other embodiments, suitable carriers include, but are not limited to, mineral oil, sorbitan monostearate, polysorbate 60, cetyl esters wax, cetearyl alcohol, 2-octyldodecanol, benzyl alcohol, water and penetration enhancers.

For ophthalmic use, compositions can be formulated as micronized suspensions in isotonic, pH-adjusted sterile saline, or, preferably, as solutions in isotonic, pH-adjusted sterile saline, either with or without a preservative such as benzylalkonium chloride. Alternatively, for ophthalmic use, the compositions can be formulated in an ointment such as petrolatum.

Compositions can also be administered by nasal aerosol or inhalation, Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents.

The compositions can be in the form of a sterile injectable preparation, for example, as a sterile injectable aqueous or oleaginous suspension. This suspension can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable solvents that can be employed are mannitol, water, Ringer's solution and isotonic sodium chloride solution. In addition, sterile, fixed oils are conventionally employed as a solvent or suspending medium. For this purpose, any bland fixed oil can be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms can also be used for the purposes of formulation.

The amount of a therapeutic agent(s) that can be combined with the carrier materials to produce a composition in a single dosage form will vary depending upon the host treated, the particular mode of administration and the activity of the agent employed. Preferably, compositions should be formulated so that a dosage of from about 0.01 mg/kg to about 100 mg/kg body weight/day of the agent can be administered to a subject receiving the composition.

The desired dose may conveniently be administered in a single dose or as multiple doses administered at appropriate intervals such that, for example, the agent is administered 1, 2, 3, 4, 5, 6 or more times per day. The daily dose can be divided, especially when relatively large amounts are administered, or as deemed appropriate, into several, for example 2, 3, 4, 5, 6 or more, administrations.

It should also be understood that a specific dosage and treatment regimen for any particular subject will depend upon a variety of factors, including the activity of the specific agent employed, the age, body weight, general health, sex, diet, time of administration, rate of excretion, drug combination, the judgment of the treating physician and the severity of the particular disease being treated. The amount of a therapeutic agent(s) in the composition will also depend upon the particular therapeutic agent(s) in the composition.

The exact amount of a therapeutic agent in a composition required to achieve an effective amount may vary from subject (e.g., a human subject) to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses may include different or substantially the same amounts of a therapeutic agent, such as a compound described herein. In certain embodiments, when multiple doses are administered to a subject or applied to a tissue or cell, the frequency of administering the multiple doses to the subject or applying the multiple doses to the tissue or cell is three doses per day, two doses per day (e.g. BID), one dose per day (e.g., QD), one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

Compositions, complexes and agents described herein may, for example, be administered parenteral or nonparenteral means, e.g., by injection, intravenously, intraarterially, intraocularly, intravitreally, subdermally, orally, buccally, nasally, transmucosally, topically, in an ophthalmic preparation, or by inhalation, in a dosage ranging from about 0.5 mg/kg to about 100 mg/kg of body weight or, alternatively, in a dosage ranging from about 1 mg/dose to about 1000 mg/dose, every 4 to 120 hours, or according to the requirements of the particular therapeutic agent. In some embodiments, a composition is administered from about 1 to about 6 (e.g., 1, 2, 3, 4, 5 or 6) times per day or, alternatively, as an infusion (e.g., a continuous infusion) or bolus. The amount of therapeutic agent that may be combined with a carrier material to produce a single dosage form may vary depending upon the host treated and the particular mode of administration. In some embodiments, a composition comprises from about 5% to about 95% therapeutic agent(s) (w/w). In other embodiments, a composition comprises from about 20% to about 80% therapeutic agent(s) (w/w).

In some embodiments, dose ranges described herein provide guidance for the administration of provided compositions to an adult. The amount to be administered to, for example, a child or an adolescent, may be determined by a medical practitioner or person skilled in the art and may be lower or the same as that administered to an adult.

In some embodiments, a method of the present disclosure (e.g., a method of treating a vascular disease in a subject in need thereof) comprises administering to a subject in need thereof a) sphingosine-1-phosphate, a derivative thereof, or a pharmaceutically acceptable salt thereof, bound to a protein, and b) heparin, a derivative thereof, or a pharmaceutically acceptable salt thereof.

### Kits

Also encompassed by the disclosure are kits (e.g., pharmaceutical packs). In some embodiments, a kit comprises a composition of the present disclosure. The kits provided may comprise: a complex, wherein the complex comprises sphingosine-1-phosphate, a derivative thereof, or a pharmaceutically acceptable salt thereof, bound to a protein; and heparin, a derivative thereof, or a pharmaceutically acceptable salt thereof (e.g., heparin sodium), or a conjugate of the present disclosure.

In some embodiments, a kit comprises: the complex; and heparin or a pharmaceutically acceptable salt of heparin. In some embodiments, a kit comprises: the complex; and heparin sodium.

The kits may further comprise a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound contained in the kit. In some embodiments, the pharmaceutical composition or compound described herein provided in the first container and the second container are combined to form a unit dosage form.

Thus, in one aspect, provided are kits including a first container comprising:
a complex, wherein the complex comprises sphingosine-1-phosphate, a derivative thereof, or a pharmaceutically acceptable salt thereof, bound to a protein; and heparin, a derivative thereof, or a pharmaceutically acceptable salt thereof,
or a conjugate of the present disclosure.

In certain embodiments, the kits are useful in one or more of the methods described herein, for example, for treating a disease (e.g., a vascular disease) in a subject (e.g., a human subject) in need thereof. In certain embodiments, the kits are useful for preventing a disease in a subject in need thereof. In certain embodiments, the kits are useful for reducing the risk of developing a disease in a subject in need thereof.

A kit described herein may include one or more additional therapeutic agents described herein as a separate composition or in a combination comprising a complex (e.g., a complex comprising sphingosine-1-phosphate, a derivative thereof, or a pharmaceutically acceptable salt thereof, bound to a protein) and/or a compound (e.g., heparin, a derivative thereof, or a pharmaceutically acceptable salt thereof) of the present disclosure, a conjugate of the disclosure, or pharmaceutical composition thereof. In some embodiments the components of a kit are packaged in separate containers. In some embodiments, at least some of the components are combined in one or more containers.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information.

A composition (or formulation) disclosed herein may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

### EXAMPLES

### Example 1

### Abbreviations

AF—Alexa Fluor; Ang—Angiopoietin; BS—Blocking solution; BSA—Bovine serum albumin; CD68—Cluster of differentiation 68; CILS—Chemical Imaging of Living Systems; DAPI—4′,6-diamidino-2-phenylindole; DF—Disturbed flow; EC—Endothelial cell; EDG—G protein-coupled cell surface receptors; eNOS—Endothelial-type nitric oxide synthase; EXTL3—Exostosin-like glycosyltransferase-3; GA—Glutaraldehyde; GAGs—Glycosaminoglycans; GCX—Glycocalyx; Gi—Inhibitory guanine nucleotide regulatory protein; GPC1—Glypican-1; HCAECs—Human coronary artery endothelial cells; HS—Heparan sulfate; ICAM-1—Intercellular adhesion molecule-1; IV—Intravenous injection; KLF2—Krüppel-like Factor 2; KLF4—Krüppel-like Factor 4; LCA—Left carotid artery; MFI—Mean fluorescent intensity; MPA—Median pairwise alignment; NO—Nitric oxide; NU-IACUC—Northeastern University Institutional Animal Care and Use Committee; OOP—Object orientation parameter; PBS—Phosphate buffered solution; p-eNOS—Phosphorylated endothelial nitric oxide synthase; PFA—Paraformaldehyde; PI3K—Phosphoinositide 3-kinase; RCA—Right carotid artery; S1P—Sphingosine-1-phosphate; S1PR1—S1P receptor-1; SDC1—Syndecan-1; SEM—Standard error of the mean; shRNA—Short hairpin RNA; TNF-α—Tumor necrosis factor-alpha; UF—Uniform flow; VCAM-1—Vascular cell adhesion molecule-1; WGA—Wheat germ agglutinin; ZO-1—Zonula occluden-1

### Materials & Methods

### Overview

Studies presented in this disclosure involve both in vitro and in vivo experiments that each utilize unique complex models that incorporate two flow regions. There is an atheroprone DF region characterized by recirculation and stagnation. In addition, there is an atheroprotective UF region characterized by unidirectional laminar shear stress. The in vitro and in vivo effects of DF and UF on endothelial GCX expression and endothelial mechanotransduction were assessed, with the expectation of impaired GCX and endothelial function in DF compared to UF conditions. Co-treatment of albumin-bound S1P and heparin were administered in vitro and in vivo to test therapeutic efficacy in reversing GCX damage and restoring proper endothelial function, including barrier function and vascular tone. The mechanism of action of the co-treatment was investigated with a focus on potential mechanical (fluid and particle simulation) and chemical (receptor targeting and biosynthesis of glycosaminoglycans) factors.

### Disturbed Flow (DF) Models:

Both in vitro and in vivo atherosclerotic-prone DF models were used to accelerate endothelial dysfunction, a hallmark and initiator of atherosclerotic disease [17]. Specifically, a parallel-plate flow chamber cell culture model [11,16] and a partial ligation of left carotid artery (LCA) murine model [3,5,44] were used to induce acute DF patterns in vitro and in vivo, respectively. Both models also have adjacent atheroprotective UF patterns to mitigate sample preparation and the number of cell culture and animal samples.

Parallel-Plate Flow Chamber (Shear Stress Apparatus) In Vitro: Human coronary arterial ECs (HCAECs) purchased from PromoCell (Heidelberg, Germany) were cultured between passages 4 and 8 in PromoCell Endothelial Cell Growth Medium MV2 supplemented with growth factors, fetal calf serum, and antibiotic penicillin streptomycin. ECs were grown and maintained in a sterile humidified incubator at 37° C. with 5% CO₂. For shear stress experimental use, HCAECs were seeded at a density between 21,000-31,000 cells/cm² on human protein-derived fibronectin (Gibco™) (60 g/mL) coated glass coverslips. HCAECs were then transferred to the flow chamber system with PromoCell Endothelial Cell Growth Medium MV2 supplemented with 0.5% bovine serum albumin (BSA) circulating through the chamber. This exposed the HCAECs to dynamic shear stress [11,16,44]. The chamber was designed to simulate atheroprone DF patterns upstream, characterized by fluid recirculation, stagnation, and directional gradient, with an eventual downstream recovery to unidirectional 12 dynes/cm² atheroprotective UF patterns. 12-hour flow conditioned HCAECs were compared to HCAECs at 0-hour baseline conditions (these cells were acclimated in BSA-containing experimental medium for 30 minutes) and HAECs left in static conditions for concurrent 12 hours. Therefore, three different cell culture groups were examined: 1) 0-hour static (baseline EC behavior to which data was normalized); 2) 12-hour static; and 3) 12-hour flow (includes both UF and DF conditions).

Partial Carotid Ligation Murine Model In Vivo: All animal studies were conducted under protocol 23-0716R, approved by the Northeastern University Institutional Animal Care and Use Committee (NU-IACUC). 10-12-week-old male C57Bl/6 mice were obtained from Jackson Laboratories and were housed in Northeastern University's animal facility in a standard laboratory environment with an ambient temperature of 22-25° C., humidity of 55-65%, and a 12/12 hour light/dark cycle. Animals were acclimated to these conditions and fed a regular chow diet and water ad libitum for at least 1 week as recommended by NU-IACUC. For experiments, all mice were subjected to partial ligation surgery of their left carotid artery (LCA) to induce acute disturbed blood flow patterns and accelerate endothelial dysfunction and GCX degradation, as previously described [3,5,43-45]. The right carotid artery (RCA) was left intact to provide a reference vessel in each mouse. A single subcutaneous injection of Meloxicam (5 mg/mL) was administered immediately before surgery for pain relief. Upon post-surgery recovery, mice were returned to the animal care facility, where standard chow diet and water were provided ad libitum. Mice were monitored daily to ensure proper recovery. For experiments, the groups examined were: 1) ligated LCA (for DF conditions); versus 2) non-ligated RCA (for UF conditions).

### HS and Albumin-Bound S1P Co-Treatment Administration:

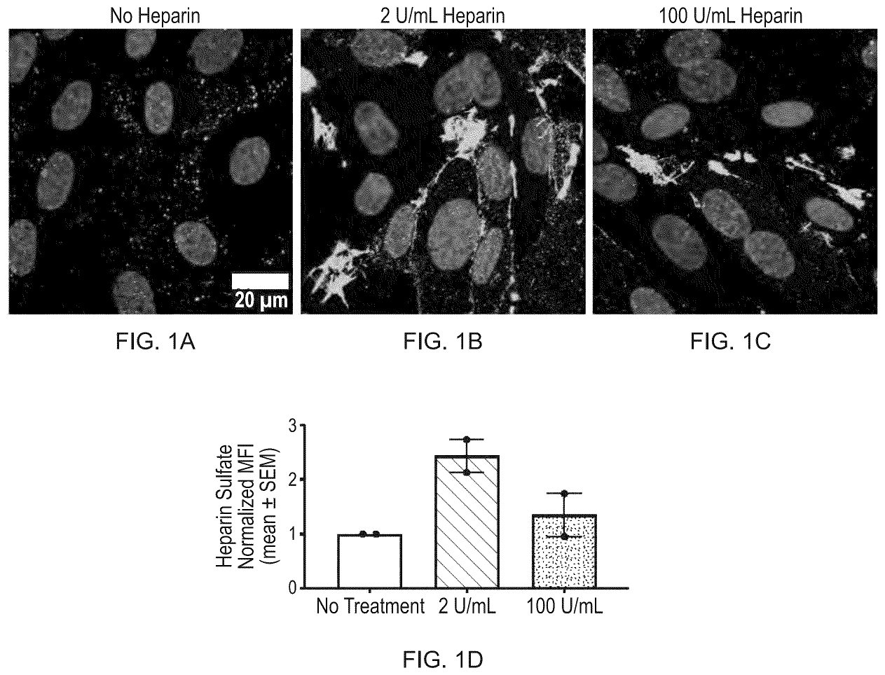

In Vitro Co-Treatment Administration: HCAECs were either not treated or exposed to co-treatment for the 12-hour duration of the flow. The treatment included heparin (2 U/mL as shown in FIG. 1B; Sigma Aldrich) and albumin-bound S1P (1.5 uM; Avanti Polar Lipids), supplemented into MV2 media with 0.5% BSA. As prior literature suggests that heparin can impair GCX barrier properties [46], an investigation was conducted to identify a safe and effective dosage. It was determined that a lower dose of heparin (2 U/mL) has therapeutic benefits in comparison to a higher dose of heparin (100 u/mL) (see FIG. 1), leading to selection of 2 U/mL of heparin for the in vitro studies. Albumin-bound S1P was prepared using the protocol suggested by Avanti Polar Lipids. C17-S1P (1 mg; Avanti Polar Lipids) was resuspended in 13.4 mL of methanol (organic solvent) for 12 hours and dried under vacuum. Fresh albumin-bound S1P solution was made before each experiment by adding 0.4% solution of PBS-Albumin (fatty acid free; Sigma) to dried S1P and subjected to a quick bath sonication.

In Vivo Co-Treatment Administration: 5 days post-LCA ligation surgery (as described above), the mice were either not treated or administered vehicle only (saline) or co-treatment (albumin-bound S1P combined with heparin). A 15 μmol/L stock solution of C17-S1P (Avanti Polar Lipids) was prepared with 4% mouse serum albumin in sterile saline, as previously described [33]. A mixture of 1.5 μmol/L albumin-bound S1P (˜150 μL) and 500 U/kg Heparin (˜10-12 μL) was created and delivered to the mouse via intravenous retro-orbital injection with a sterile 31-gauge ultra-fine needle U100 insulin syringe. Due to the approximate 15-minute half-life of albumin-bound S1P and the approximate 30-minute half-life of heparin [33,47], at 30 minutes after therapy injection, the mice were imaged via ultrasound and sacrificed to collect their vessel tissue for further processing.

### Antibody Details and Specifications

Details of all antibodies presented in this disclosure are described in Table 1. Table 1: Comprehensive summary of all antibodies used in this disclosure.

| Antibody or Lectin | | | | | |
|---|---|---|---|---|---|
| Secondary/Tertiary/Quaternary Labels | Use | Supplier | Catalog No. | Clone | Dilution |
| CD68 rat anti-mouse IgG₂ₐ | macrophage | Bio-Rad | MCA1957GA | FA-11 | 1: 250 |
| (monoclonal) ^{h, b, j} | in vivo | | | | |
| eNOS rabbit IgG (monoclonal) e | total eNOS | Cell | 32027S | D9A5L | 1: 500 |
| | label in vitro | Signaling | | | |
| | | Tech | | | |
| EXTL3 rabbit anti-human IgG | EXTL3 label | Fisher | PIPA5112818 | n/a | 1: 100 |
| (polyclonal) ^{f, j} | in vivo | Scientific | | | |
| Glypican-1 mouse IgG₁ | GPC1 in | Santa Cruz | SC-365000 | A-10 | 1: 100 |
| (monoclonal) ^{c, a} | vitro | Biotech | | | |
| Glypican 1 rabbit IgG | GPC1 in | Invitrogen | PA5-28055 | n/a | 1: 200 |
| (polyclonal) ^{f, j} | vivo | | | | |
| Heparan Sulfate mouse IgM | heparan | Amsbio | 370255-1 | F58- | 1: 100 |
| (monoclonal) ^{d} | sulfate in | | | 10E4 | |
| | vitro | | | | |
| Phosphorylated eNOS (Serine | p-eNOS in | Cell | 9571S | n/a | 1: 500 |
| 1171) rabbit ^{e} | vitro | Signaling | | | |
| | | Tech | | | |
| S1PR1 mouse anti-human IgG₃ | S1PR1 in | Santa Cruz | SC-48356 | A-6 | 1: 100 |
| (monoclonal) ^{c, a} | vitro | Biotech | | | |
| S1PR1 rat anti-mouse IgG₂ₐ | S1PR1 in | R&D | MAB7089 | 713412 | 1: 200 |
| (monoclonal) ^{g, j} | vivo | Systems | | | |
| Syndecan-1/CD138 rabbit IgG | SDC1 in | Novus | NBP2-67174 | JM11- | 1: 100 |
| (monoclonal) ^{e} | vitro and in | Biologicals | | 21 | |
| | vivo | | | | |
| Wheat Germ Agglutinin | whole GCX | VectorLabs | B10255 | n/a | 1: 200 (in |
| (WGA), Biotinylated ^{a} | in vitro and | | | | vivo) |
| | in vivo | | | | 1: 50 (in |
| | | | | | vivo) |

| | | | Catalog | |
|---|---|---|---|---|
| | Secondary/Tertiary/Quaternary Labels | Supplier | Number | Dilution |
| a | Steptavidin, Alexa Flour 488 Conjugated | Jackson | 016-540-084 | 1:400 to 1:500 |
| | | Laboratories | | |
| b | Steptavidin, HRP Conjugated | Thermofisher | N100 | 1:250 to 1:500 |
| | | Scientific | | |
| c | Goat Anti-Mouse IgG, IgM (H + L) | Invitrogen | 31802 | 1:400 |
| | Cross-Absorbed, Biotin Conjugated | | | |
| d | Goat Anti-Mouse IgG, IgM (H + L) | Bio-Rad | MCA1957GA | 1:250 |
| | Alexa Fluor 488 Conjugated | | | |
| e | Goat Anti-Rabbit IgG (Heavy Chain) | Invitrogen | PIA27034 | 1:200 to 1:400 |
| | Superclonal, Alexa Flour 488 | | | |
| | Conjugated | | | |
| f | Goat Anti-Rabbit IgG (H + L), HRP | Invitrogen | 65-6120 | 1:250 |
| | Conjugated | | | |
| g | Goat Anti-Rat IgG (H + L) Secondary | Thermofisher | 31470 | 1:500 |
| | Antibody, HRP Conjugated | Scientific | | |
| h | Goat Anti-Rat IgG Antibody (H + L), | VectorLabs | BA-9400 | 1:200 |
| | Biotin Conjugated | | | |
| i | Rabbit Anti-Rat IgG (H + L) mouse- | VectorLabs | BA-4001 | 1:200 |
| | absorbed, Biotin Conjugated | | | |
| j | TSA Plus, Cyanine 3 Conjugated | Akoya Biosciences | NEL744001KT | 1:100 |

### Staining of Endothelial GCX GAGs, Core Proteins, and S1P Receptor-1:

Immediately after exposure to different stimulus conditions and treatments, HCAECs and mouse tissue were stained to probe different biomarkers and assess response to heparin and albumin-bound S1P co-treatment vs. no treatment conditions. These biomarkers included whole GCX labeled with WGA lectin, which binds to multiple GCX components including sialic acid and N-acetyl-D glucosamine residues of HS and hyaluronic acid [48]. Additional biomarkers included, HS (most abundant GAG of the GCX), GCX core protein syndecan-1 (SDC1; binds to HS and chondroitin sulfate), and GCX core protein glypican-1 (GPC1; binds to HS). The S1P receptor 1 (S1PR1; specifically present on endothelial cells) was also stained.

In situ biomarker staining was chosen as an optimal approach to study EC GCX, as it preserves the structural integrity of the GCX while allowing for the visualization of its distribution, thickness, and other physical characteristics. Protein quantification methods like Western blotting and flow cytometry are unsuitable for this purpose. GCX consists primarily of sugar chains rather than proteins [29,49]. For Western blotting, the sample preparation process extracts these sugar chains along with their associated proteins, resulting in a nonspecific smear across molecular weight ranges. Flow cytometry requires cells to be in suspension, which compromises the cell membrane and disrupts the surface GCX. In contrast, fixing cell cultures and tissue samples, followed by fluorescent labeling, maintains the GCX structure and enables precise quantification of relative expression while avoiding cross-contamination of cells from DF and UF regions, especially in small regions of DF within the cell culture model [11].

In Vitro Methods: Descriptions of methods applied to HCAECs are described in Table 2.

Table 2. Staining Protocols for GCX Components and S1PR1 (in vitro). Additional abbreviations described herein are as follows: Ab—antibody, AF—Alexa Fluor, BS—blocking serum, BSA—bovine serum albumin, GA—glutaraldehyde, PBS—phosphate buffered saline, PFA—paraformaldehyde, WGA—wheat germ agglutinin. Please note that the protocols outlined in this table only apply in vitro and do not apply in vivo.

| | | Permeabil- | Blocking | Primary | | | |
|---|---|---|---|---|---|---|---|
| Biomarker | Fixative | ization | Solution (BS) | Antibody (Ab) | Secondary Ab | Tertiary Ab | Mounting Medium |
| Heparan | 2% PFA + | n/a | 2% goat serum | 1:100 HS | 1:400 AF488 | n/a | VectaShield (Vector |
| Sulfate | 0.1% GA | | in PBS (1 hour) | antibody, | goat anti- | | Labs) anti-fade |
| | (30 minutes) | | | 10e4 epitope | mouse IgM in | | mounting medium |
| | | | | in BS, 4° C. | BS (30 minutes) | | with 4′,6-diamidino- |
| | | | | (2-3 days) | | | 2-phenylindole |
| Whole GCX | | | 3% BSA in | 1:200 | 1:500 AF488 | | (DAPI) |
| (WGA) | | | PBS (1 hour) | Biotinylated | streptavidin in | | |
| | | | | WGA Lectin in | BS (30 minutes) | | |
| | | | | BS (1 hour) | | | |
| Syndecan-1 | | 0.1% Triton | 5% goat serum | 1:100 SDC1 | 1:400 AF488 | | |
| | | in PBS | in PBS (1 hour) | (CD138) anti- | goat anti- | | |
| | | (10 minutes) | | rabbit | rabbit IgG | | |
| | | | | monoclonal | (1:400) in BS | | |
| | | | | in BS, 4° C. | (2 hours) | | |
| | | | | (2-3 days) | | | |
| Glypican-1 | 2% PFA | 0.1% Triton | 5% BSA in | 1:100 GPC1 | 1:400 | 1:400 AF488 | |
| | (30 minutes) | in PBS | PBS (1 hour) | mouse | Biotinylated | Streptavidin | |
| | | (5 minutes) | | monoclonal | goat anti- | in PBS (1 | |
| | | | | antibody in | mouse in BS | hour) | |
| | | | | BS, 4° C. | (1 hour) | | |
| | | | | (3 days) | | | |
| S1PR1 | | | | 1:100 S1PR1 | | 1:400 AF488 | |
| | | | | mouse | | Streptavidin | |
| | | | | monoclonal | | in PBS (1.5 | |
| | | | | antibody in | | hours) | |
| | | | | BS (3 days) | | | |

In Vivo Methods: Before processing the mouse tissue, an extensive process of LCA and RCA tissue dissection, preservation, and cryosectioning was implemented, as previously described [3,5].

Subsequently, to assess GCX integrity in situ, immunohistochemical analysis was performed on sectioned LCA and RCA using WGA lectin and SDC1 antibody, according to a prior published protocol [3].

Similar approaches, with minor modifications, were used to stain GPC1 and S1PR1. Previously perfusion-fixed tissue samples on glass slides were post-fixed in 4% PFA for 10 minutes and then washed thoroughly with PBS. Tissue samples were then permeabilized for 10 min with 0.3% Triton X-100 diluted in PBS. Subsequently, antigen retrieval was achieved by heating samples in 10 mM sodium citrate at a pH of 6.0 in PBS (for 10 min) and allowing them to equilibrate to room temperature. Next, endogenous peroxidase blocking was achieved by using a 1% hydrogen peroxide solution diluted in deionized water for 30 min with rocking. Tissue samples were then introduced to another blocking solution (10% goat serum and 0.3% Triton) for 1 hour in a humidified condition. Slides were then incubated with either polyclonal rabbit GPC1 antibody (Invitrogen; 1:200 in blocking solution) for 3 days or clone #713412 monoclonal rat IgG_{2A} antibody against mouse S1PR1 (a.k.a. S1P₁/EDG-1 antibody; from R&D Systems; 1:200 in blocking solution) for 2 days. Following primary antibody incubation, tissue samples were exposed to either a goat anti-rabbit secondary antibody (for GPC1) or a goat anti-rat secondary antibody (for S1PR1) conjugated to horse radish peroxidase (1:250 dilution in PBS for GPC1; 1:500 in PBS for S1PR1) and incubated for 1 hour in a humidified condition at 4 C. Tissue samples were subsequently washed in a 0.1% Tween 20 (Sigma-Aldrich Co.) solution in PBS. A TSA Cyanine 3 amplification system kit from Akoya Biosciences (Part No. NEL704A001KT) was then used to incubate tissue samples for 5 min. Finally, the slides were washed in 0.1% Tween 20 in PBS.

Stained tissue samples were mounted and sealed using VectaShield anti-fade mounting medium with DAPI for cell nuclei staining (VectorLabs, part number H1200).

### Assessing Co-Treatment Effect on Endothelial Cell and Vascular Function

General Methods: Vascular tone regulation and remodeling, as well as barrier function, were monitored to assess the co-treatment's ability to reverse endothelial dysfunction in vitro and in vivo. To assess vascular tone regulation in vitro, HCAECs were stained for serine 1177 phosphorylated endothelial nitric oxide synthase (p-eNOS; active form) and total eNOS. Furthermore, remodeling of cultured HCAEC morphology was assessed (round versus elongated in the direction of flow). To assess vascular tone and remodeling in vivo, periodic ultrasound data was collected to determine diastolic and systolic lumen diameter and vessel wall thickness of the LCA and RCA. Macrophage uptake via CD68 was assessed for hyperpermeability in vivo. Further details of studies are described below:

Monitoring In Vitro Cellular Remodeling, As Indicated by EC Morphology: Cell orientation and alignment analysis was done using a custom Python script applied to brightfield images of flow-conditioned HCAEC monolayer. Four regions were imaged for each condition, and each region was analyzed to obtain alignment scores. Images underwent Otsu local thresholding (radius=100), and morphological closing (erosion+dilation) to isolate cell blobs, followed by size-based filtering to exclude small specs and background regions. Ellipses were fitted to the identified blobs to extract the cells' centers, lengths, widths, and orientation angles, with 500-1000 cells analyzed per sample. Manual inspection ensured the quality of segmentation and ellipse fitting. To measure alignment, cells with an aspect ratio greater than 2 were retained, and their orientation angles were adjusted for bidirectionality. Alignment was measured in two ways: 1) Object Orientation Parameter (OOP) and 2) median pairwise alignment (MPA). The OOP score is computed as OOP=2 cos²(θ)−1, where θ is the orientation angle. OOP ranges from −1 (perpendicular to flow) to +1 (parallel to flow) [50]. MPA measures alignment of cells to each other and is computed as the median of the absolute dot products of cell orientations, where values range from 0.5 (random orientation) to 1 (perfect alignment).

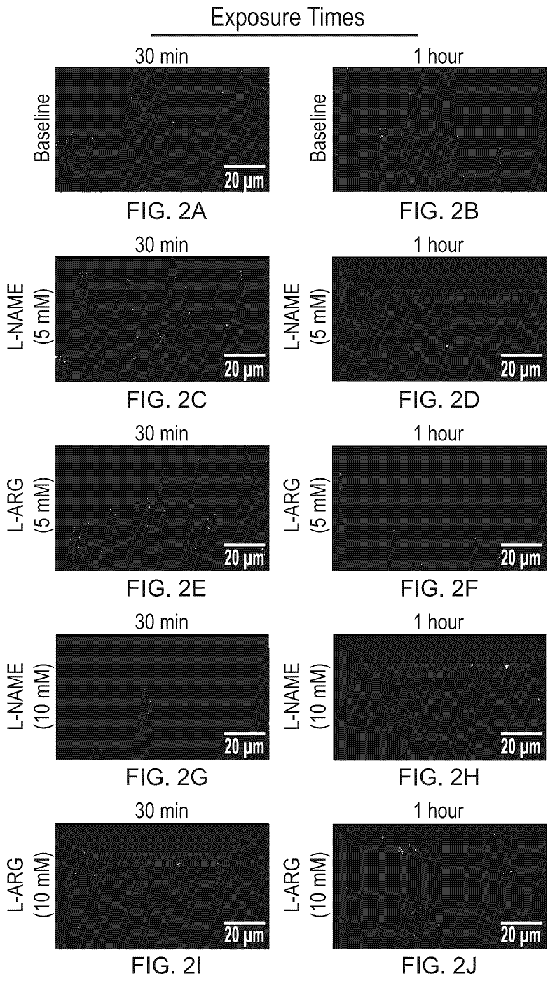

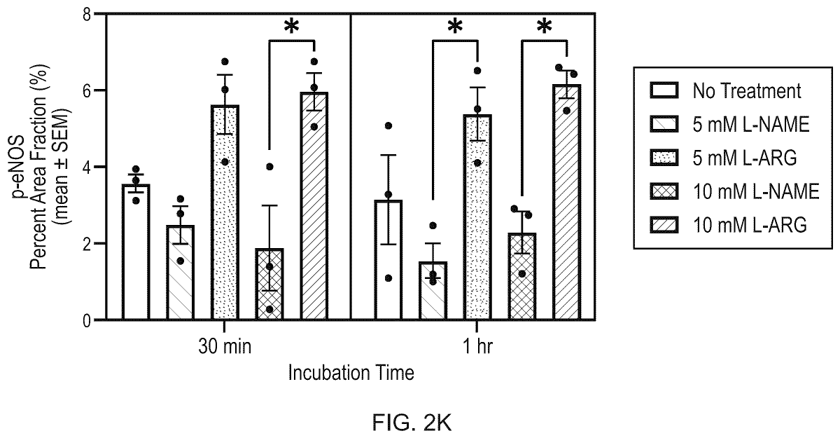

Monitoring EC Control of Vascular Remodeling Through Expression of Vascular Tone Regulators in vitro: HCAECs were first fixed in a 4% PFA solution for 20 minutes. Samples were then permeabilized in 0.5% triton in PBS solution for 15 minutes and blocked in 10% goat serum in PBS for 1 hour at room temperature. The coverslips were incubated with their respective primary antibody at a 1:500 dilution in blocking solution in a humidified chamber for 3 days at 4° C. The antibody used for the p-eNOS targeted eNOS that was phosphorylated at serine 1177, which represents the active form of eNOS. The antibody that was used to target total eNOS was the D9A5L version. After primary antibody incubation and thorough washing, samples were incubated with Alexa Fluor 488 labeled goat anti-rabbit secondary antibody (1:200) in PBS for 1 hour in dark, room temperature conditions. Once staining was completed, samples were mounted and sealed using VectaShield anti-fade mounting medium with DAPI for cell nuclei staining (VectorLabs, H1200). Pilot studies were conducted to demonstrate the accuracy of p-eNOS staining, as shown in FIG. 2.

Monitoring Vascular Remodeling Via High-Resolution Ultrasound: Blood vessel diameter and wall thickness were measured using ultrasound, which was conducted with Fujifilm Visual Sonics Vevo® 3100 LT with MX700 (29-71 MHz; 10 mm scan depth) and MX550S (25-55 MHz; 15 mm scan depth) transducers. All RCAs and LCAs of mice were periodically imaged on day 0 (before LCA ligation), day 3 (post-ligation), and day 5 (post-ligation and 30 minutes after co-treatment injection). Mice were anesthetized with inhaled isoflurane (1-2.5%), and body temperature was maintained on a heated 38° C. platform. Levels of anesthesia, heart rate, temperature, and respiration were continuously monitored during the imaging session. Pulse-wave Doppler Mode, which can detect any blood flow disturbances in the vessel or region of bifurcation, was used to assess blood flow velocity through the carotid artery to ensure partial ligation of the LCA was successful. M-mode was used for vessel dimensions. Calculated parameters include vessel wall thickness and diastolic and systolic lumen diameter. All measurements were gated to an electrocardiogram and respirator.

Monitoring In Vivo Barrier Functionality Through Vessel Wall Infiltration by Macrophages: LCA and RCA infiltration by macrophages was assessed via cluster of differentiation 68 (CD68) staining. The full protocol was previously published [3,5]. Briefly, tissue samples were blocked with 4% goat serum in PBS for 10 minutes, then incubated with primary rat anti-mouse CD68 (Bio-Rad, 1:250) in blocking solution at 4° C. for 1 hour. Subsequently, slides were incubated for 10 minutes with biotinylated goat anti-rat antibody (VectorLabs, 1:200), washed in PBS containing 0.1% Tween 20, and treated with HRP-conjugated streptavidin (1:500) in blocking solution for 1 hour. The TSA Cyanine 3 amplification kit (Akoya Sciences) was applied, followed by washes in PBS with 0.1% Tween 20, and the slides were mounted. Cyanine 3 exhibits excitation/emission maxima of ˜550/570 nm, spanning the orange-red spectrum. To better differentiate the green elastin (vessel wall) from CD68-positive areas, we used a microscope setting that converts the Cyanine 3 red color to a “false” yellow color, as published previously [3,5], ensuring accurate visualization.

### Assessment of Co-Treatment Mechanisms of Action

General Methods: The mechanism of action of the co-treatment was investigated in three ways: 1) by determining the residence time of the therapy in DF via fluid and particle simulations, 2) by determining if the co-treatment acts via the S1P receptor pathway, and 3) by determining whether there is active nascent proteoglycan synthesis via exostosin-like glycosyltransferase-3 (EXTL3).

Simulations to Assess a Potential Mechanical Mechanism of Action: The fluid flow in the millifluidic device was modeled as an incompressible Newtonian fluid with a density of

$${ρ}_{f} = 1.002 \frac{g}{{cm}^{3}}$$

and dynamic viscosity of μ=0.862 mPa·s. The steady-state velocity and pressure fields in the millifluidic device (Ω) were governed by the Navier-Stokes equations: ∇·u=0 in Ω and [u·∇]

$$u = \frac{- 1}{{ρ}_{f}} ∇ p + \frac{μ}{{ρ}_{f}} {∇}^{2} u$$

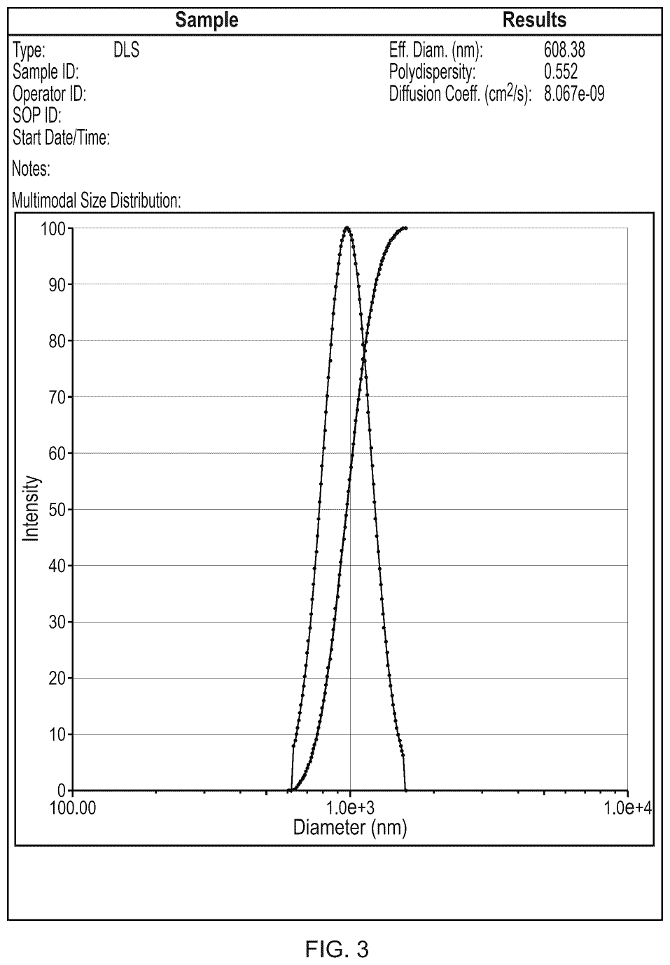

in Ω, where u represents the fluid velocity vector, p represents the pressure, and pf represents fluid density. The steady-state Navier-Stokes equations were solved using the simpleFoam solver through the open-source computational fluid dynamics software OpenFOAM. The millifluidic parallel-plate flow device can hold up to four glass coverslips to increase experimental throughput. The flow chamber consists of 4 individual “steps” to ensure each coverslip is exposed to atheroprone DF conditions upstream of atheroprotective UF conditions. Initial simulations demonstrated identical flow fields over each “step” within the millifluidic device. Thus, it was optimal to simulate fluid and particle transport over only one “step”. The computational domain was discretized into 9.9 million orthogonal elements using OpenFOAM's blockMesh utility. The inlet was assigned a constant flux Dirichlet boundary condition of 102 mL/min, the outlet was assigned a zero-pressure Neumann boundary condition, and the walls were assigned a no-slip boundary condition. Following fluid simulations, the transport of co-treatment of albumin bound S1P and heparin was simulated through OpenFOAM's Lagrangian particle tracking solver, particleFoam. Particles were assumed not to influence the flow field nor interact with other particles. Particles were also assumed to be well-mixed throughout the medium and thus were initialized uniformly throughout the domain of interest. The particle's position is updated through the equation

$${m}_{p} \frac{{du}_{p}}{dt} = {F}_{g} + {F}_{D}$$

where uₚ is the particle velocity, mₚ is the particle mass, F_{g} is the gravitation force, and F_{D} is the drag force. Based on experimental data obtained via dynamic light scattering (see FIG. 3), all particles were assigned to have a diameter of dₚ=730 nm with a density of

$${ρ}_{p} = 2.18 \frac{g}{{cm}^{3}} .$$

Furthermore, all particles were assumed to be spherical. When a particle's center was within one radius of the wall of the microfluidic device, it was assumed to stick to the wall and was no longer tracked.

Assessing Potential Mechanism of Action Involving the S1P Receptor Pathway. To evaluate the potential involvement of S1PR1 pathway activity in vascular ECs subjected to heparin and albumin-bound S1P co-treatment, we stained cultured ECs and tissues from LCA and RCA for S1PR1. See in vitro and in vivo methods described under “Antibody Details and Specifications” and Table 2.

Assessing Potential Mechanism of Action Involving Biochemical Synthesis of GCX Replacement Components: Exostosin-like glycosyltransferase-3 (EXTL3) plays an important role in the chain polymerization of HS and heparin via the Golgi apparatus [51]. Anticipating that the heparin and albumin-bound S1P co-treatment could function through upregulation of EXTL3 activity within vascular ECs, we stained LCA and RCA tissue for EXTL3. Fixed tissue samples were treated with 10% goat blocking serum and 0.3% Triton. Tissue samples were then incubated with EXTL3 polyclonal antibody (diluted 1:100 in blocking solution) for 3 days at 4° C. Following primary antibody incubation, tissue samples were coated with 1:250 dilution in PBS of HRP-conjugated goat anti-rabbit antibody for 1 hour. TSA Cyanine 3 amplification system kit was then used to incubate the tissue samples for 5 minutes. After washing thoroughly in 0.1% Tween 20 solution, all tissue samples were mounted and sealed using VectaShield anti-fade mounting medium with DAPI for cell nuclei staining (VectorLabs, H1200).

While this disclosure incorporates both vessel tissue and cell analysis as complementary approaches, the staining protocol for EXTL3 in cell culture was not included here. Vessel tissue analysis was prioritized to highlight EXTL3 expression in its most physiological context. Future studies can include cell culture-based EXTL3 staining.

### Confocal Microscopy Imaging

All samples (except where otherwise indicated above) were imaged using a LSM 800 confocal microscope (Carl Zeiss Meditec AG, Jena, Germany) housed in the Institute for Chemical Imaging of Living Systems (CILS) core facility at Northeastern University. HCAEC samples were imaged at 63× (with an oil immersion lens) magnification with z-stack, while murine LCA and RCA samples were imaged at 20× tile scans and 40× magnification. An excitation wavelength of 360 nm was used to capture DAPI-labeled cell nuclei, and a 554 nm wavelength showed the Cyanine 3 fluorescently amplified labels of biomarkers in mice tissue samples. Additionally, 488 nm wavelength was used to visualize elastin auto-fluorescence (vessel wall) in mice carotid artery samples and to capture fluorescently tagged biomarkers of interest in HCAECs.

### Image Analysis and Data Acquisition

ImageJ was used to calculate the mean fluorescence intensity (MFI) from 63× en face images to determine overall expression levels of HS in HCAECs in vitro [11]. Percent area fractions were computed to quantify WGA, p-eNOS, total eNOS, GPC1, SDC1, and S1PR1 biomarkers of interest. Three different 63× z-stack images were taken from each sample and were averaged. Data from each run was normalized to their respective zero-hour experimental group. Phase contrast images were captured and analyzed using a customized Python script to quantify cell alignment from six experimental runs from each HCAEC treatment group.

For in vivo experiments, GCX coverage and thickness probed via labeling WGA, SDC1, and GPC1 in the LCA and RCA, as well as barrier function probed by labeling macrophage uptake within vessel walls, were imaged and analyzed via ImageJ as previously described [3,5]. EXTL3 images were analyzed by determining the percent area fraction of expression within the endothelium. For all biomarkers in vivo, three serial tissue rings were quantified and then averaged for one data point per LCA or RCA of each animal. Quantifications of various biomarkers were normalized to their RCA counterpart. Lastly, ultrasound image data was collected and analyzed via software Vevo Lab 5.6.1.

### Statistics

All data sets (raw and normalized data) were represented as mean±standard error of the mean (SEM) with a minimum of 4 independent experiments per condition, upon which significance testing in the accompanying figures were conducted. Comparisons among more than two groups were either conducted using Ordinary one-way ANOVA with Tukey's multiple comparisons test, or two-way ANOVA with Tukey's multiple comparisons test, using GraphPad Prism software (version 9.5.0). The results were also plotted using GraphPad Prism, where an α-value of 0.05 and a 95% confidence interval were used for statistical significance. In data figures, asterisks indicate statistical significance of the data, as follows: *p<0.05, **p<0.01, ***p<0.001, and ****p<0.0001.

### Results

Co-Treatment Impact on EC GCX Integrity: This portion of the results section highlights the impact that co-treatment of albumin-bound S1P and heparin has on EC GCX integrity in vitro and in vivo. The in vitro markers of the EC GCX include WGA, HS, SDC1, and GPC1. The in vivo markers of the EC GCX include WGA, SDC1, and GPC, with HS being omitted because the HS antibody is made in mouse and produces significant non-specific staining.

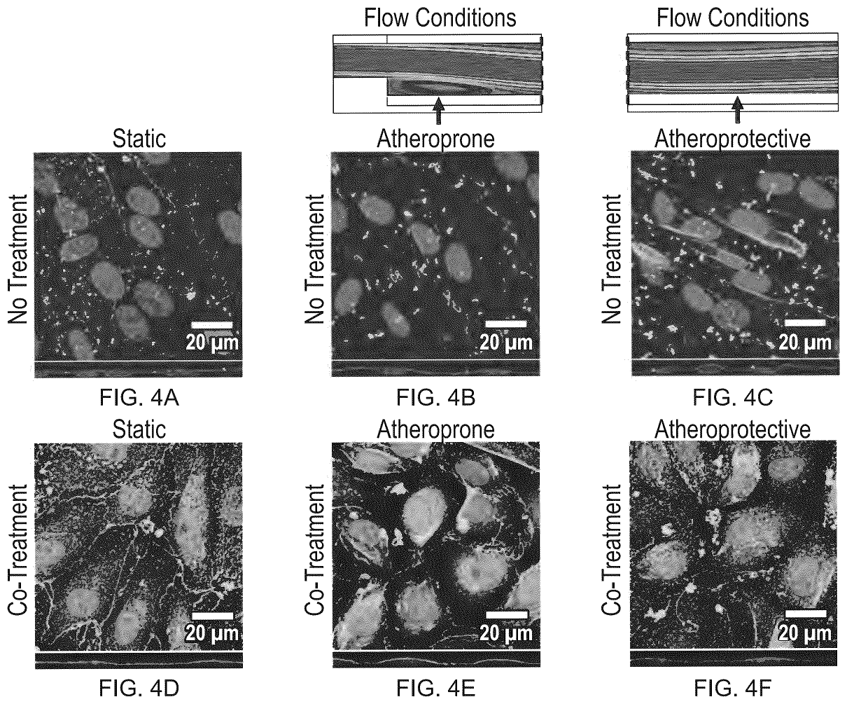

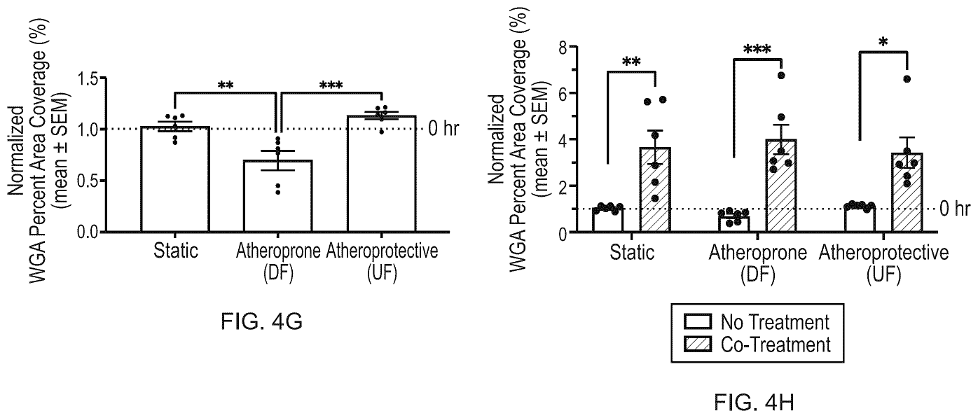

Co-Treatment increases WGA-labeled whole GCX in DF-atheroprone region in vitro and in vivo: FIG. 4 displays representative images of HCAECs stained for WGA (labeled as green) across experimental groups and data corroborating previous studies performed using parallel-plate flow chambers [11,16,44]. We assessed 12-hour flow-induced HCAEC coverage by GCX labeled using WGA lectin. Data was normalized to a 0-hour baseline within each experiment to account for variation. Under static conditions, untreated HCAECs showed a normalized WGA-labeled whole GCX expression of 1.03±0.05. In DF conditions, this decreased to 0.70±0.09; in UF conditions, it increased slightly to 1.13±0.04. DF conditions showed a significant 32% decrease compared to static conditions and a 38% decrease compared to UF conditions. No significant difference was observed between static and UF conditions. Upon exposure to co-treatment, WGA-labeled GCX expression significantly increased. In DF conditions, co-treatment resulted in a normalized WGA-labeled whole GCX expression of 3.96±0.63, compared to 0.70±0.09 for untreated DF conditions. In UF conditions, co-treatment showed a normalized expression of 3.39±0.66, compared to 1.13±0.04 for untreated UF conditions. In static conditions, co-treated HCAECs expressed 3.39±0.66, compared to 1.03±0.05 for untreated conditions. The full statistical analysis for normalized WGA percent area coverage of HCAECs among the different cohorts can be found in Table 3.

Table 3. Full statistical analysis of normalized WGA percent area coverage of HCAECs among the different cohorts. Selected results from this analysis are highlighted in the plots shown in FIG. 4.

| Normalized WGA | | | |
|---|---|---|---|
| Percent Area | | | |
| Coverage | Significance? | Summary | Adjusted P Value |
| Static: No Treatment | Yes | ** | 0.0068 |
| vs. Static: Co- | | | |
| Treatment | | | |
| Static: No Treatment | Yes | ** | 0.0051 |
| vs. DF: No Treatment | | | |
| Static: No Treatment | Yes | ** | 0.0018 |
| vs. DF: Co-Treatment | | | |
| Static: No Treatment | No | ns | >0.9999 |
| vs. UF: No Treatment | | | |
| Static: No Treatment | Yes | * | 0.0165 |
| vs. UF: Co-Treatment | | | |
| Static: Co-Treatment | Yes | ** | 0.0019 |
| vs. DF: No Treatment | | | |
| Static: Co-Treatment | No | ns | 0.9960 |
| vs. DF: Co-Treatment | | | |
| Static: Co-Treatment | Yes | * | 0.0101 |
| vs. UF: No Treatment | | | |
| Static: Co-Treatment | Yes | ns | 0.9992 |
| vs. UF: Co-Treatment | | | |
| DF: No Treatment vs. | Yes | *** | 0.0005 |
| DF: Co-Treatment | | | |
| DF: No Treatment vs. | Yes | *** | 0.0005 |
| UF: Co-Treatment | | | |
| DF: No Treatment vs. | Yes | ** | 0.0047 |
| UF: Co-Treatment | | | |
| DF: Co-Treatment vs. | Yes | ** | 0.0028 |
| UF: Co-Treatment | | | |
| DF: Co-Treatment vs. | No | ns | 0.9569 |
| UF: Co-Treatment | | | |
| UF: No Treatment vs. | Yes | * | 0.0240 |
| UF: Co-Treatment | | | |

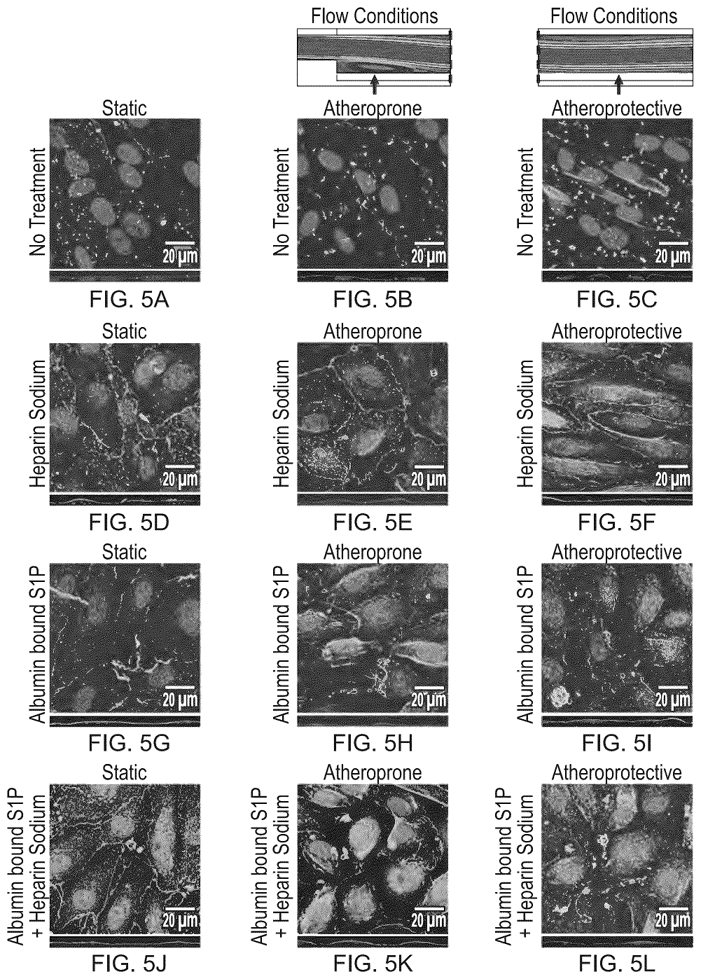

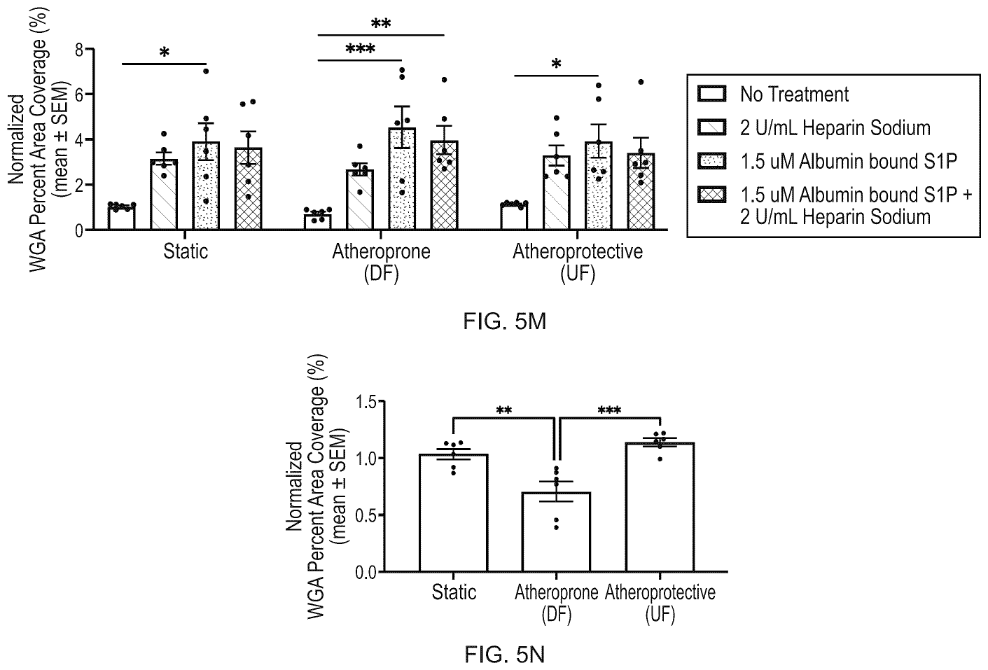

FIG. 5 contains results that show WGA-labeled GCX expression in HCAECs that were treated with albumin-bound S1P or heparin separately.

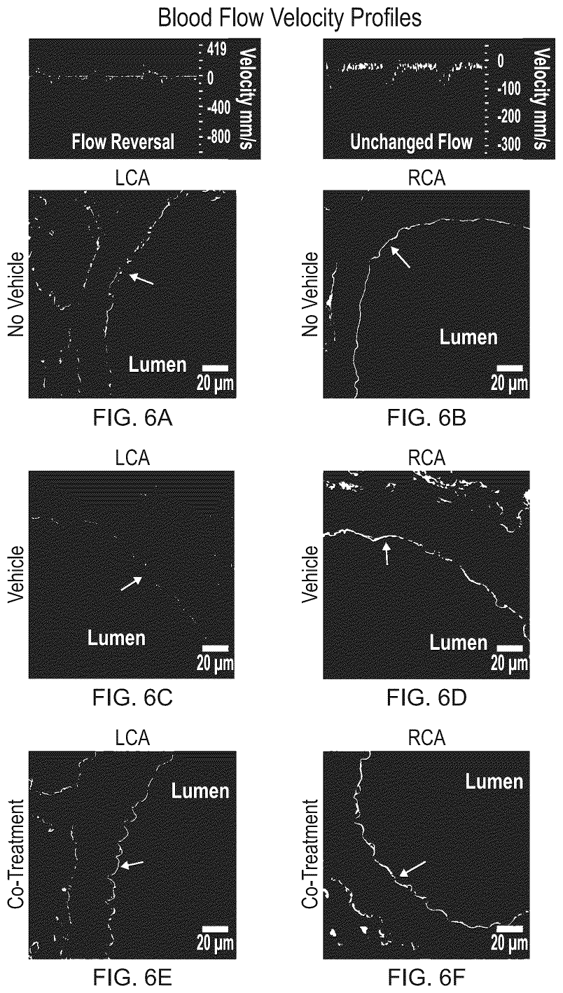

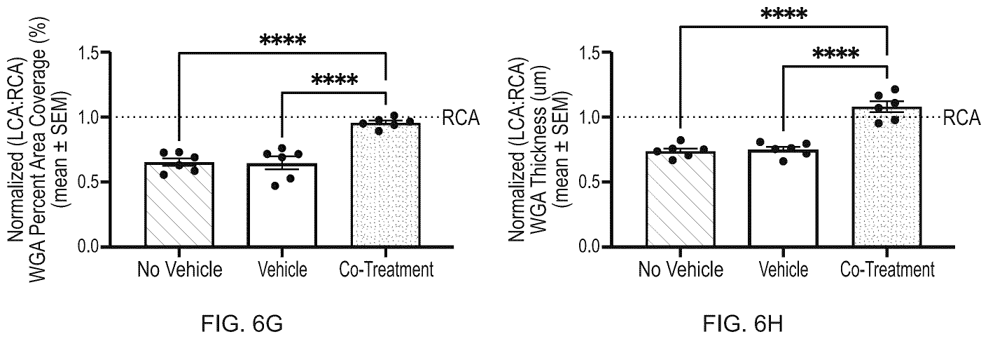

FIG. 6 presents in vivo data validating the in vitro (FIG. 4) findings. As previously published, in untreated conditions the WGA-labeled GCX on the non-ligated RCA in C57BL/6 male mice expresses a WGA fluorescence pattern that spans 76.3±10.2% of the inner blood vessel wall [44], In the present, disclosure, the WGA (labeled as red) fluorescence pattern was found to present even higher RCA coverage of 86.6±1.40% with vehicle treatment, 89.2±1.03% with co-treatment of albumin-bound S1P and Heparin, and 92.2±0.64% in untreated conditions. The ligated LCA, normalized to the RCA per condition, showed a normalized vessel coverage of 0.65±0.03 in untreated conditions, 0.65±0.05 with vehicle-only, and 0.95±0.02 with co-treatment, indicating that albumin-S1P/heparin results in a significant 46% increase compared to untreated and 48% increase compared to vehicle-only conditions. GCX thickness on the RCA wall measured 1.45±0.08 μm in untreated conditions, 1.65±0.08 μm in vehicle-only, and 1.56±0.06 μm with co-treatment, with no significant differences between conditions. In the ligated LCA, thickness ranged from 0.90 μm to 2.05 μm across all conditions. When the data was normalized to the RCA, the readout is 0.74±0.02 for untreated, 0.75±0.02 for vehicle-only, and 1.08±0.04 for co-treatment conditions. Co-treatment induced a significant 46% increase compared to untreated and 44% increase compared to vehicle-only conditions, returning GCX thickness to healthy RCA levels.

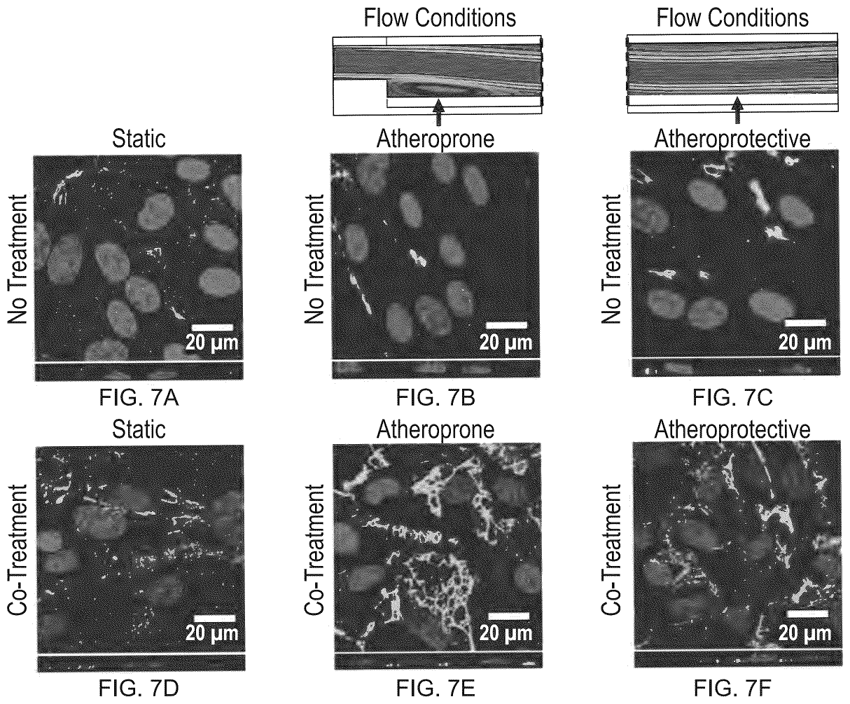

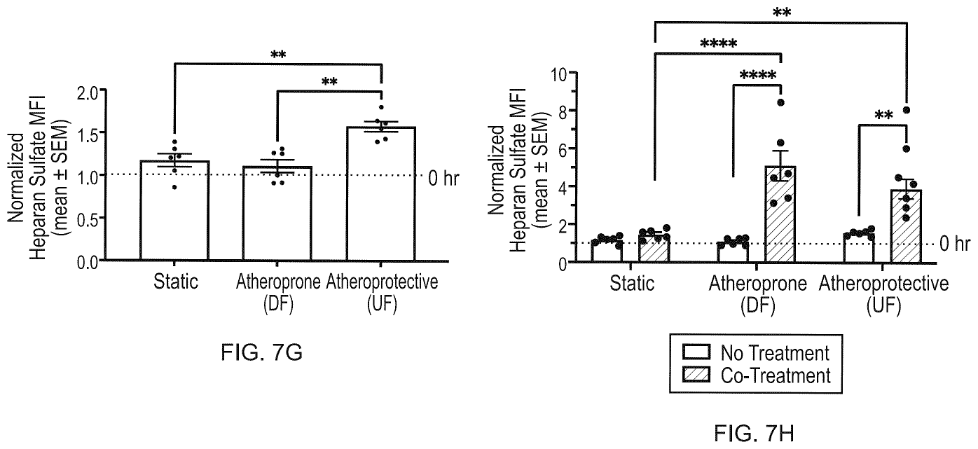

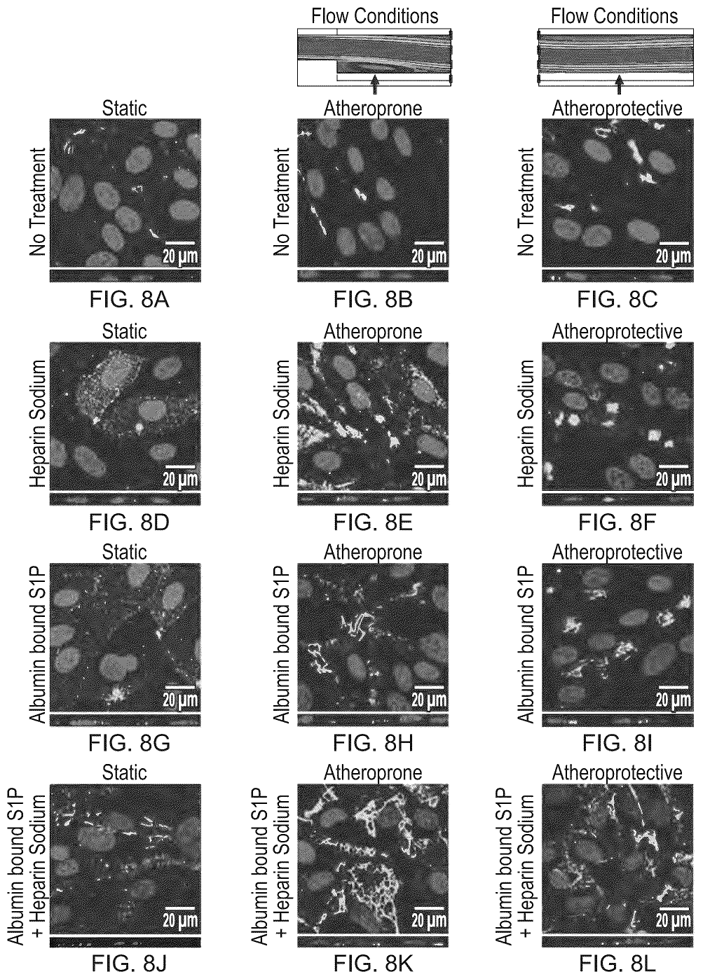

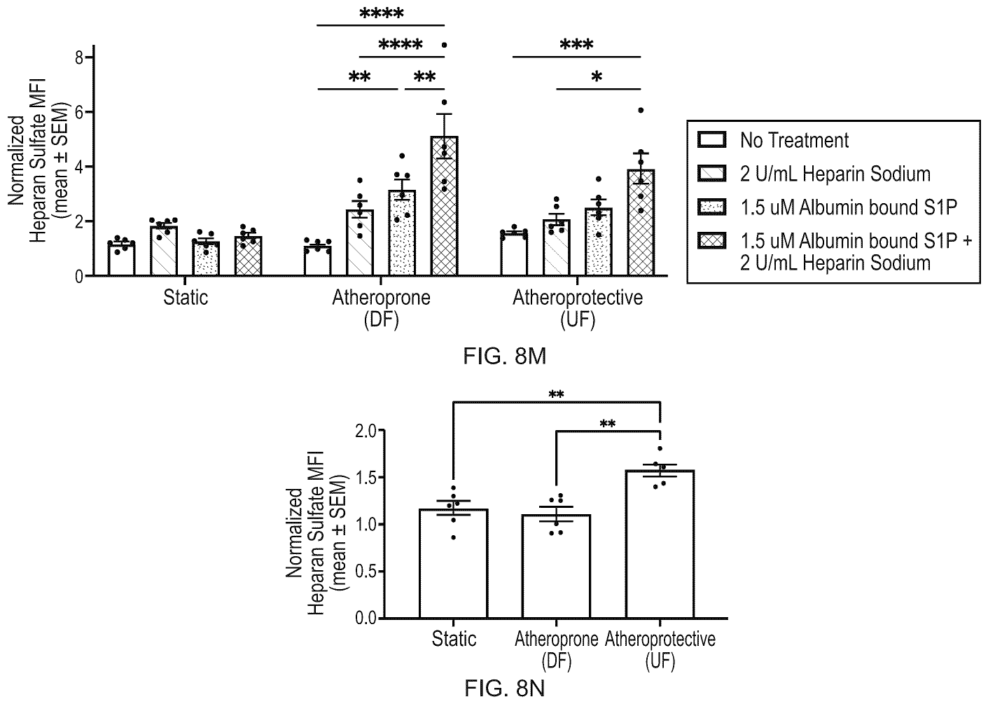

Co-Treatment increases HS expression in DF-atheroprone region in vitro: FIG. 7 shows representative images of stained HS component of the GCX on cultured HCAECs and resembles HS staining in other types of ECs [52,53]. To our knowledge, we are the first to observe HS in HCAECs. Analysis of the images was conducted using mean fluorescence intensity (MFI) quantification [11]. MFI data was normalized to a 0-hour baseline within each experiment to account for variation. Normalized HS MFI in 12-hour static HCAECs was 1.17±0.08, 12-hour DF HCAECs was 1.11±0.08, and 12-hour UF HCAECs was 1.57±0.06, which was statistically significantly high. After establishing HCAEC HS MFI expression in untreated static, DF, and UF conditions, HCAECs were exposed to co-treatment of albumin-bound S1P and heparin to determine the efficacy of HS GAG restoration. The co-treated HCAEC HS MFI showed a normalized level of 1.11±0.08 in static conditions, 5.11±0.81 in DF conditions, and 3.92±0.53 in UF conditions. Notably, the co-treatment effects were statistically significant in DF and UF conditions but not statistically significant in 12-hour static conditions. The full statistical analysis for normalized HS MFI expression by different HCAEC cohorts can be found in Table 4, while FIG. 8 shows HS expression in HCAECs treated with albumin-bound S1P or heparin separately.

Table 4. Full statistical analysis of normalized HS mean fluorescence intensity (MFI) expression by HCAECs among the different cohorts. Selected results from this analysis are highlighted in the plots shown in FIG. 7.

| Normalized Heparan | | | |
|---|---|---|---|
| Sulfate MFI | | | |
| Expression | Significance? | Summary | Adjusted P Value |
| Static: No Treatment | No | ns | >0.9999 |
| vs. Static: Co- | | | |
| Treatment | | | |
| Static: No Treatment | No | ns | >0.9999 |
| vs. DF: No Treatment | | | |
| Static: No Treatment | Yes | **** | <0.0001 |
| vs. DF: Co-Treatment | | | |
| Static: No Treatment | Yes | ** | 0.0034 |
| vs. UF: No Treatment | | | |
| Static: No Treatment | Yes | *** | 0.0006 |
| vs. UF: Co-Treatment | | | |
| Static: Co-Treatment | No | ns | >0.9999 |
| vs. DF: No Treatment | | | |
| Static: Co-Treatment | Yes | **** | <0.0001 |
| vs. DF: Co-Treatment | | | |
| Static: Co-Treatment | No | ns | >0.9999 |
| vs. UF: No Treatment | | | |
| Static: Co-Treatment | Yes | ** | 0.0024 |
| vs. UF: Co-Treatment | | | |
| DF: No Treatment vs. | Yes | **** | <0.0001 |
| DF: Co-Treatment | | | |
| DF: No Treatment vs. | Yes | ** | 0.0010 |
| UF: No Treatment | | | |
| DF: No Treatment vs. | Yes | *** | 0.0004 |
| UF: Co-Treatment | | | |
| DF: Co-Treatment vs. | Yes | **** | <0.0001 |
| UF: No Treatment | | | |
| DF: Co-Treatment vs. | No | ns | 0.5036 |
| UF: Co-Treatment | | | |
| UF: No Treatment vs. | Yes | ** | 0.0040 |
| UF: Co-Treatment | | | |

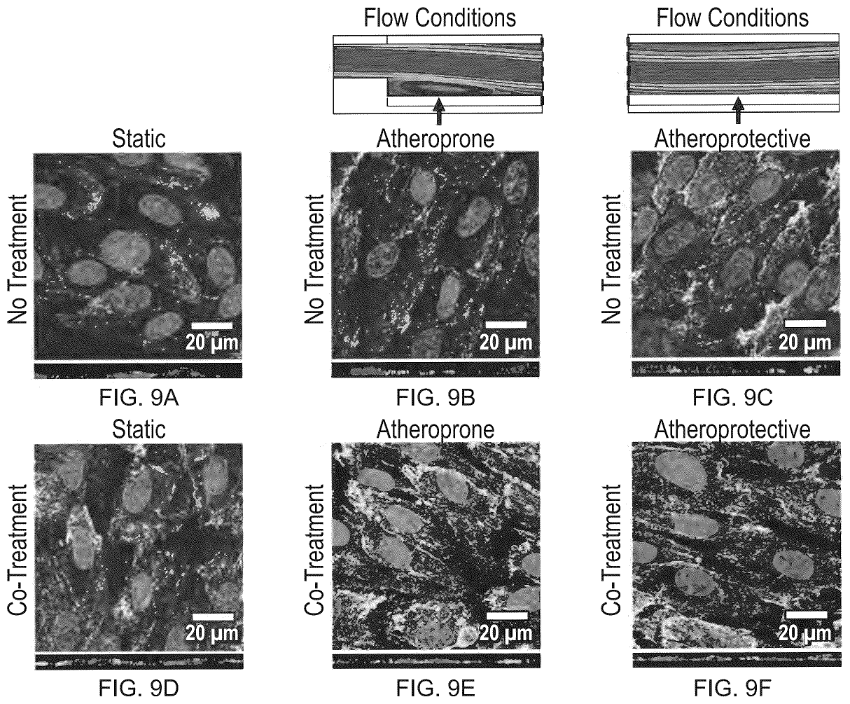

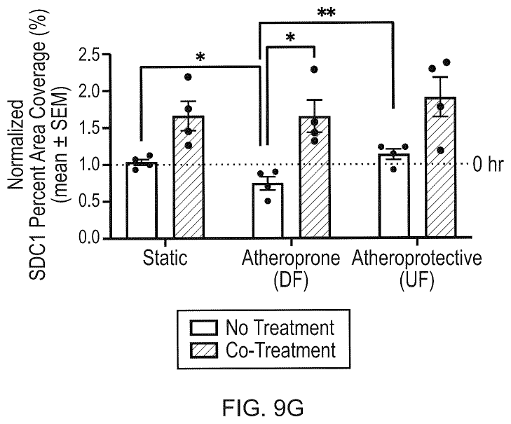

Co-Treatment increases GCX core protein expression in DF-atheroprone region in vitro and in vivo: To further assess the impact of the co-treatment on the HS component of the EC GCX, we probed the in vitro and in vivo expression of the transmembrane and cytoskeleton-linked SDC1 and membrane and lipid-raft-linked GPC1 [54]. SDC1 and GPC1 are core GCX proteins that specifically covalently bind HS [54]. FIG. 9 shows 63× z-stack representative images of SDC1 on HCAECs in vitro in different stimulus environments. Data were normalized to 0-hour baseline conditions. In no treatment conditions, normalized SDC1 percent area coverage was 1.04±0.04 in 12-hour static conditions, 0.75±0.09 in 12-hour DF conditions, and 1.14±0.07 in 12-hour UF conditions. This indicates that SDC1 was statistically significantly impaired by DF conditions compared to static or UF conditions. This is consistent with data previously reported in the literature [55]. In co-treatment conditions, normalized SDC1 percentage area increased to 1.66±0.20 in 12-hour static conditions, 1.65±0.22 in 12-hour DF conditions, and 1.90±0.275 in 12-hour UF conditions.

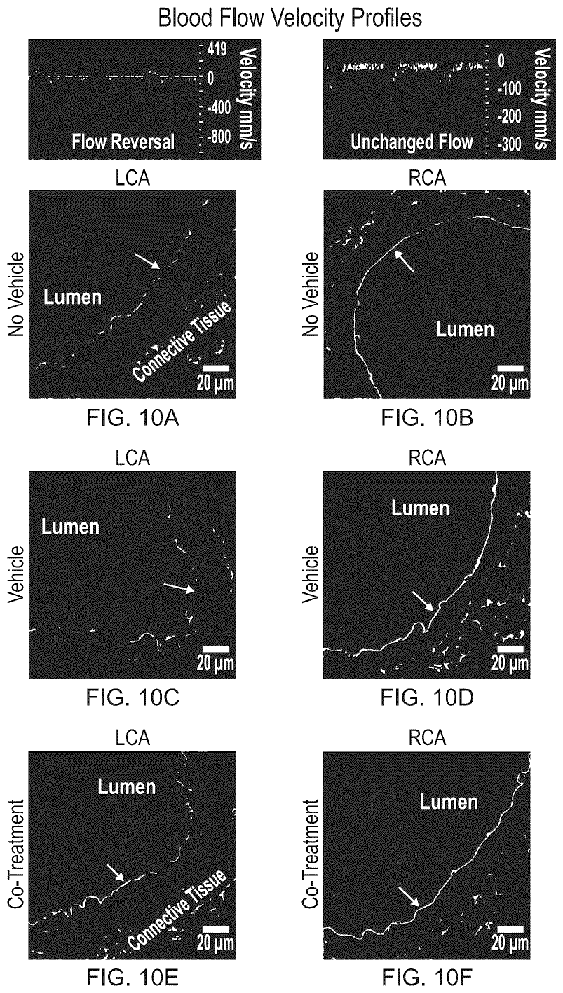

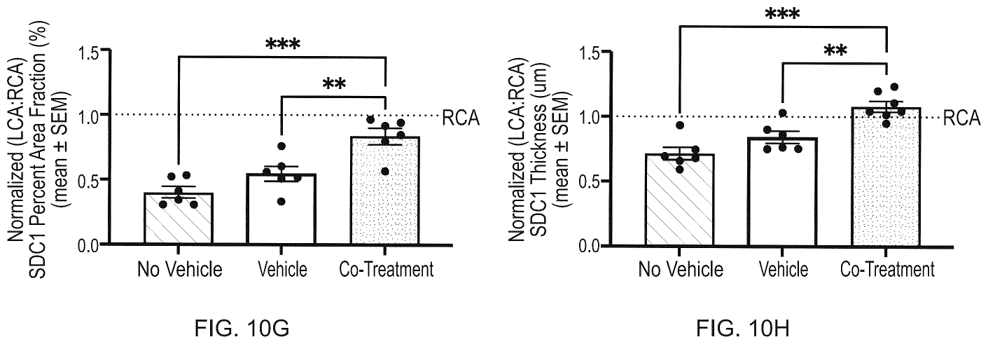

FIG. 10 shows results from the in vivo partially ligated LCA murine model, including representative core protein SDC1 (red) stained images acquired at 40× magnification. Data for ligated LCA was normalized to the RCA per condition. SDC1 percent area fraction was 0.41±0.04 in untreated (no vehicle) conditions, 0.55±0.06 in vehicle-only conditions, and 0.84±0.06 in co-treatment conditions, with co-treatment yielding statistically significantly more SDC1 expression compared to no treatment and vehicle-only. Analysis was also conducted on SDC1 transversal thickness of the ligated LCA normalized by non-ligated RCA for all mice observed: normalized SDC1 thicknesses was 0.72±0.05 in untreated (no vehicle) conditions, 0.85±0.05 in vehicle-only conditions, and most significantly significant 1.09±0.05 in co-treatment conditions.

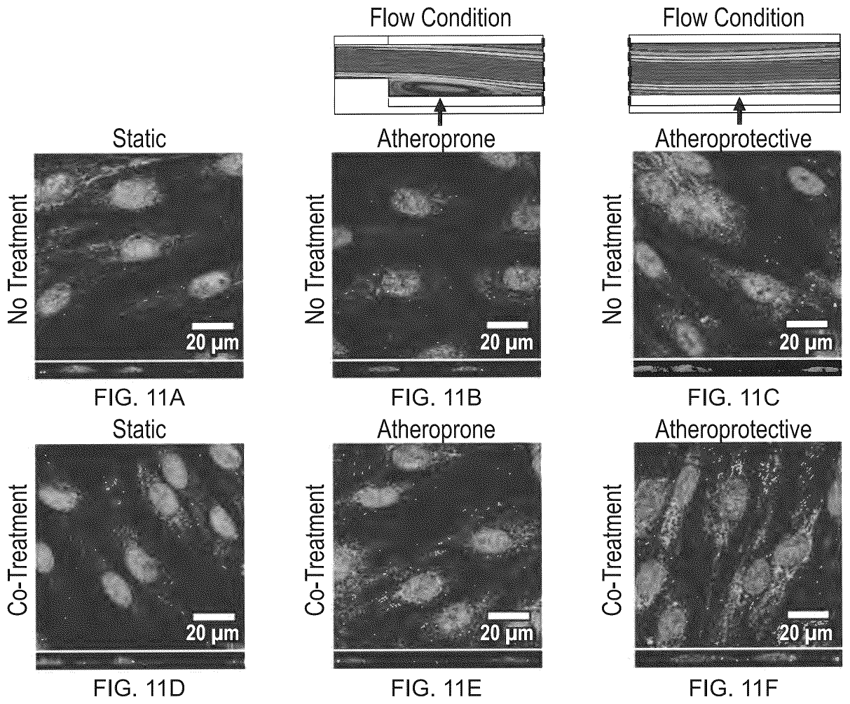

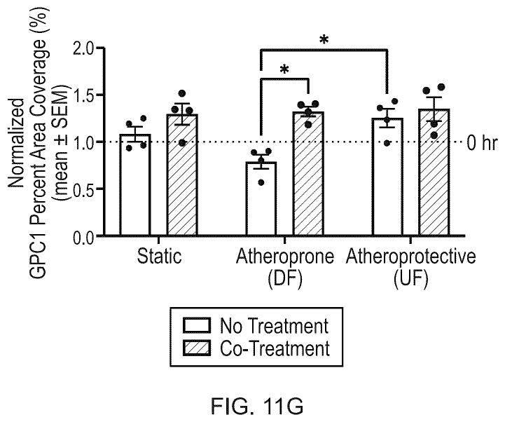

FIG. 11 shows in vitro results for GPC1, including both fluorescent (green for GPC1) images and plotted quantification of the 12-hour results normalized to 0-hour baseline conditions. Normalized GPC1 percent area coverage of untreated cultured HCAEC layers was 1.08±0.08 in 12-hour static conditions, 0.78±0.08 in 12-hour DF conditions, and 1.25±0.10 in 12-hour UF conditions. When co-treatment was introduced, GPC1 coverage at normalized levels was 1.29±0.11 for static conditions, 1.32±0.05 for DF conditions, and 1.35±0.13 for UF conditions. The only statistically significant co-treatment-induced GPC1 enhancement occurred in DF conditions with an approximate 70% increase.

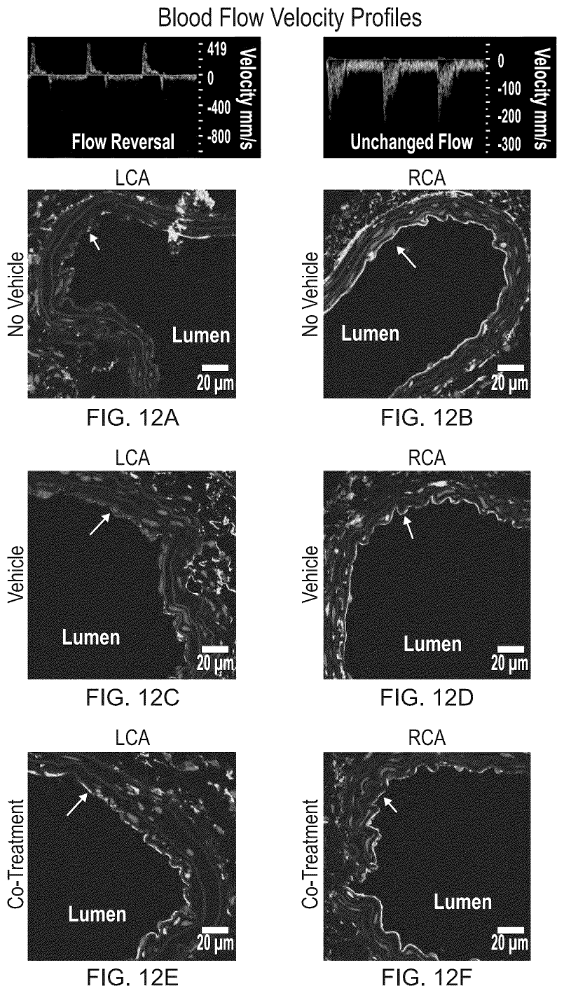

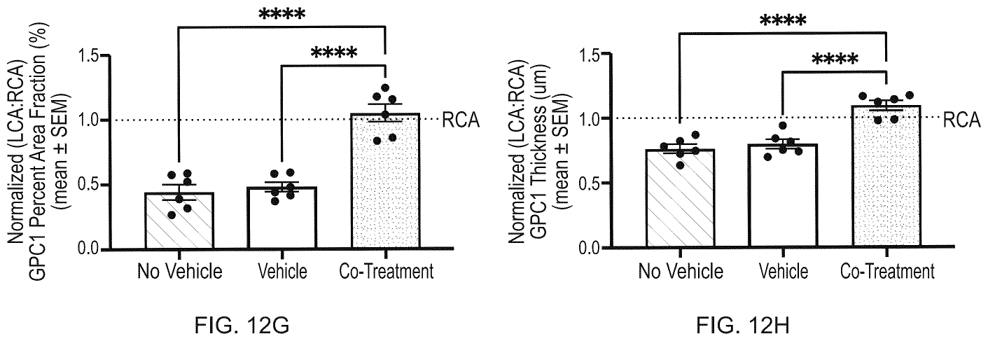

GPC1 data collected from the mouse model is shown in FIG. 12, micrographically and quantitatively. When normalized to the RCA condition, the GPC1 percent area fraction of the inner LCA wall was 0.45±0.06 in untreated (no vehicle) conditions, 0.49±0.04 in vehicle-only conditions, and 1.06±0.07 in co-treatment conditions. The co-treatment of albumin-S1P and heparin resulted in statistically significant restoration of GPC1. Normalized transversal LCA GPC1 thickness measured 0.79±0.03 in untreated (no vehicle) conditions, 0.80±0.04 in vehicle-only conditions, and 1.10±0.04 in co-treatment conditions. The co-treatment statistically significantly boosted GPC1 thickness compared to baseline conditions: 43% compared to the untreated (no vehicle) case and 37% compared to the vehicle-only case.

### Co-Treatment Impact on Endothelial and Vascular Function:

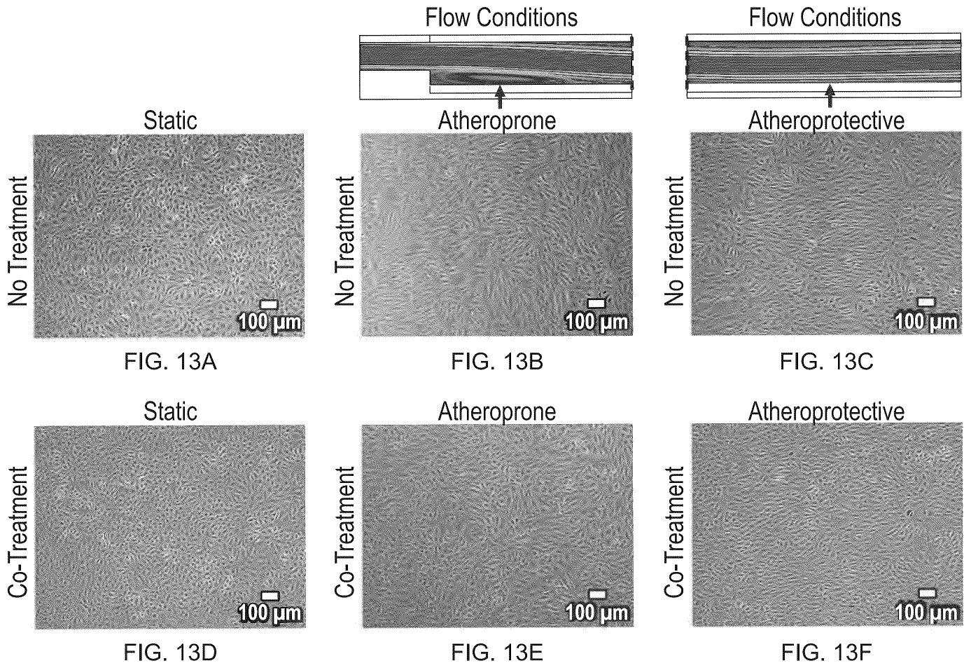

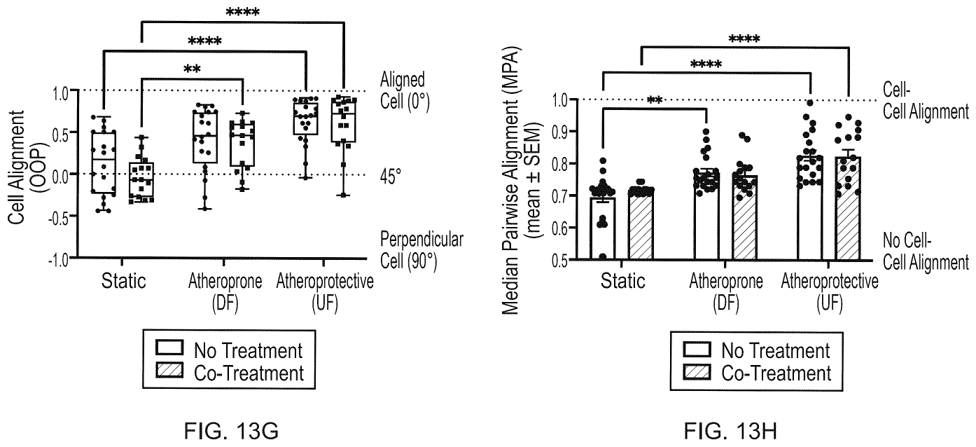

Co-treatment does not affect flow-controlled in vitro cellular remodeling and morphology; cell alignment is not enhanced under any flow condition: Co-treatment with albumin-S1P and heparin does not significantly affect flow-controlled in vitro cellular remodeling or morphology, as cell alignment was not enhanced under any flow condition. Analysis of OOP and MPA metrics (FIG. 13) showed that alignment was primarily influenced by flow conditions. Under DF conditions, HCAECs showed a trend toward alignment in the direction of flow and relative to each other, reflecting moderate organization. In contrast, UF conditions exhibited the highest alignment, with HCAECs more strongly oriented both in the direction of flow and among neighboring cells. Co-treatment did not significantly alter these metrics across any flow condition. These results suggest that within the 12-hour timeframe of this disclosure, cellular elongation and alignment are governed by the type of flow, with DF producing moderate alignment and UF resulting in the strongest alignment, rather than by co-treatment effects.

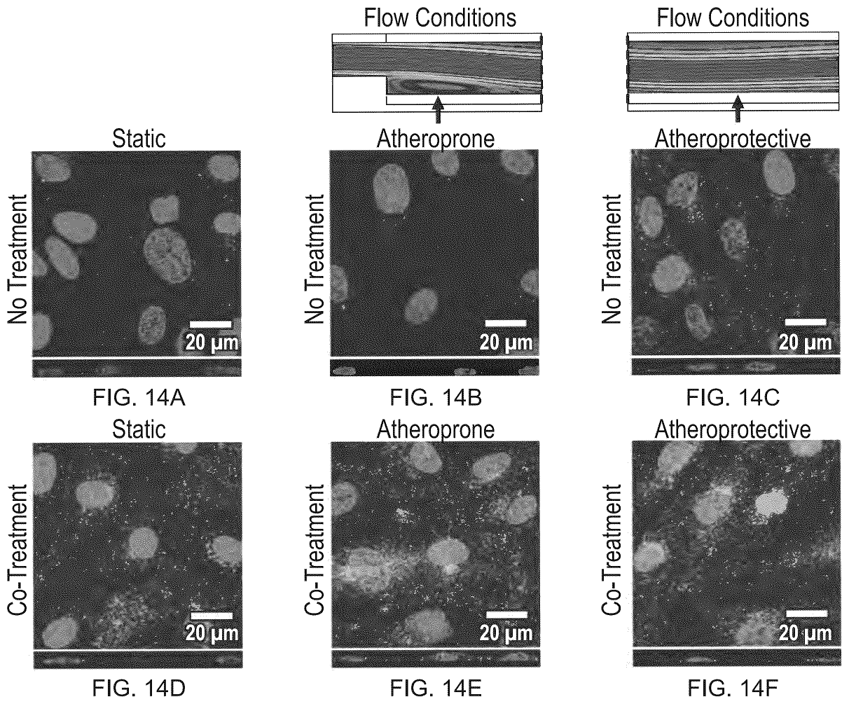

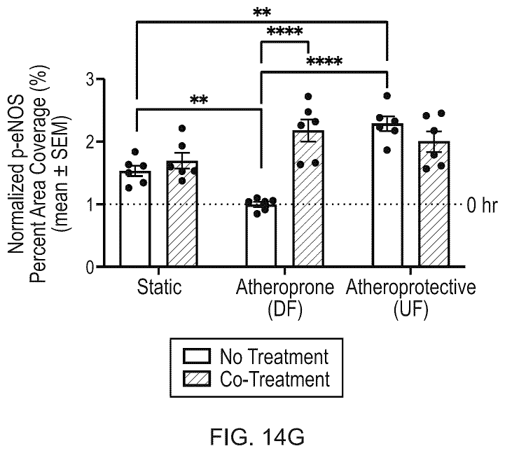

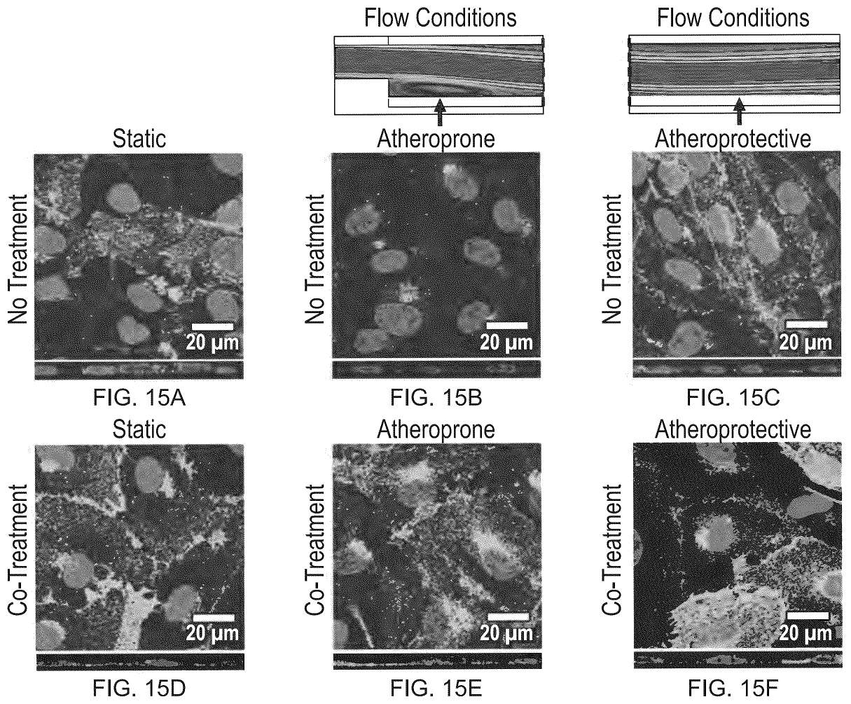

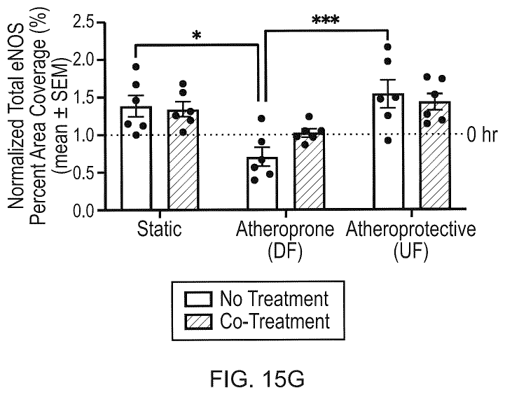

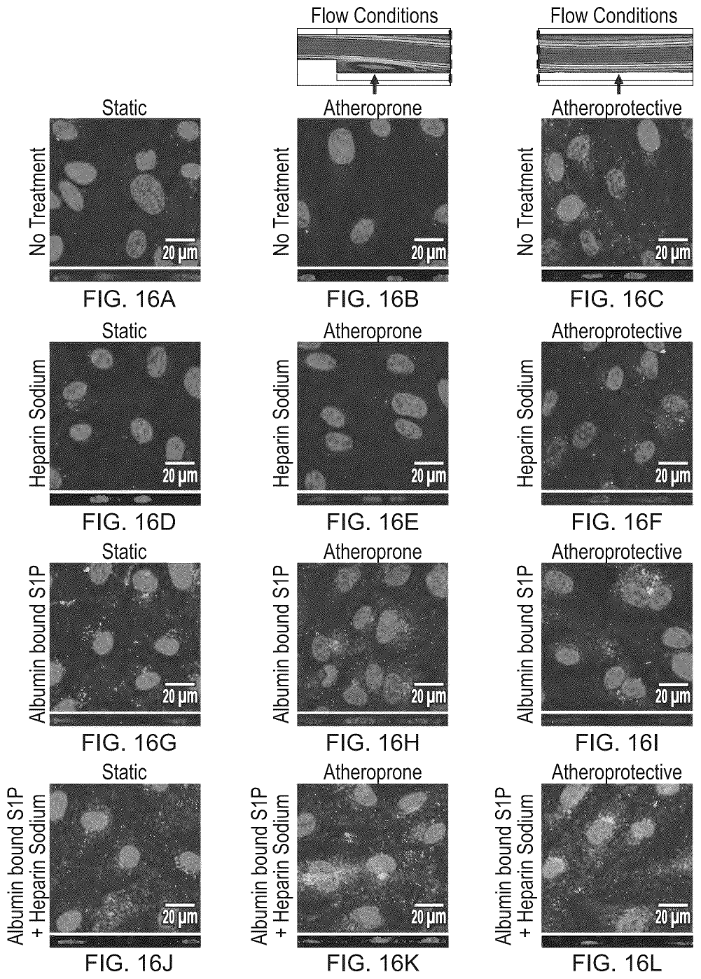

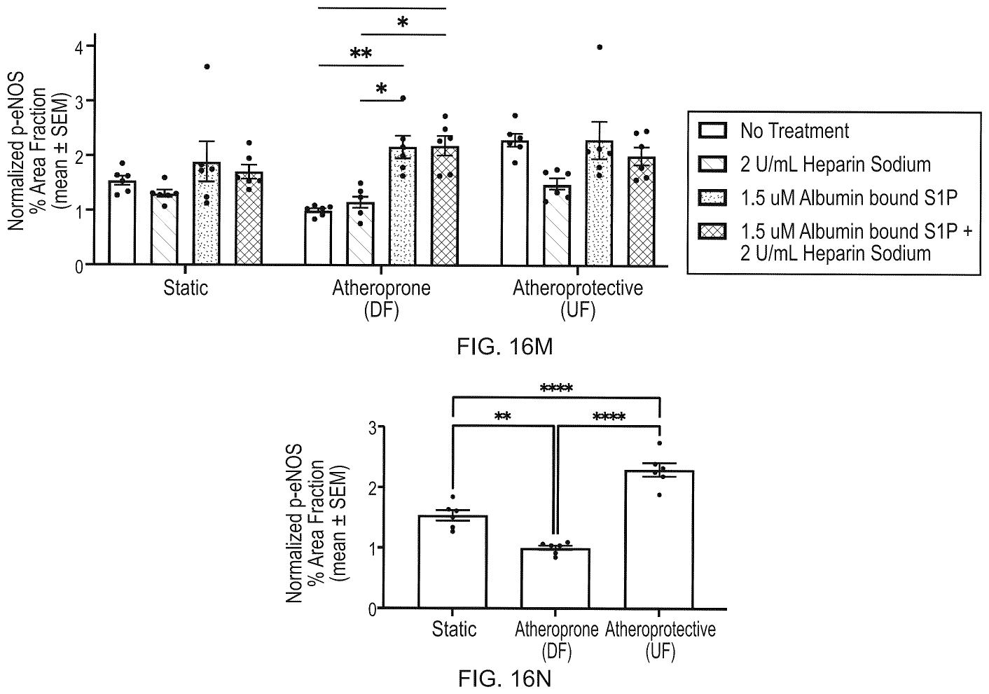

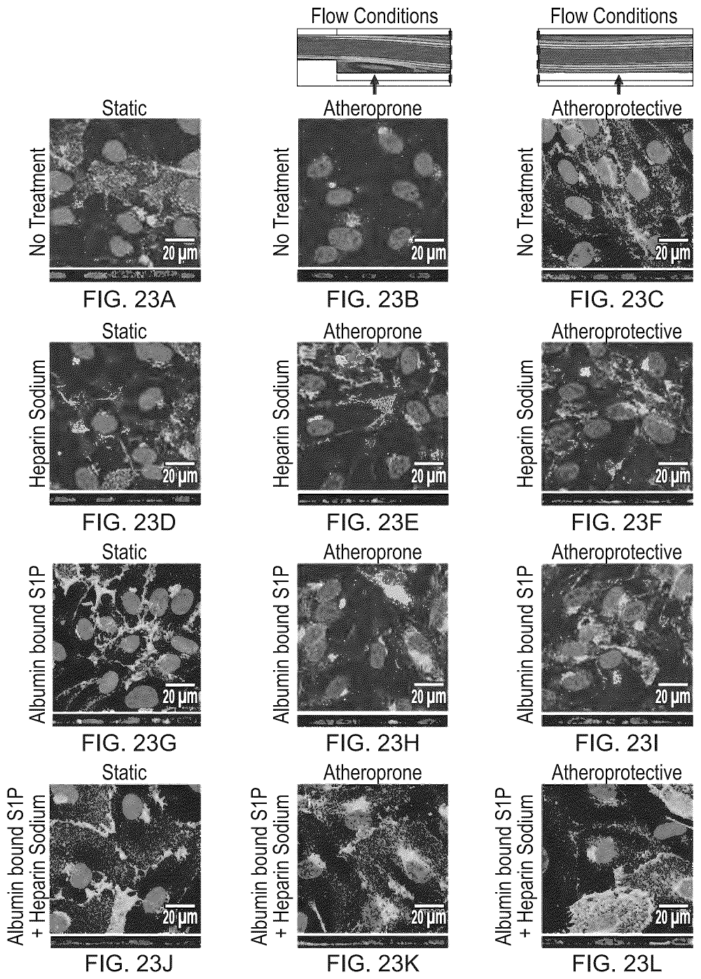

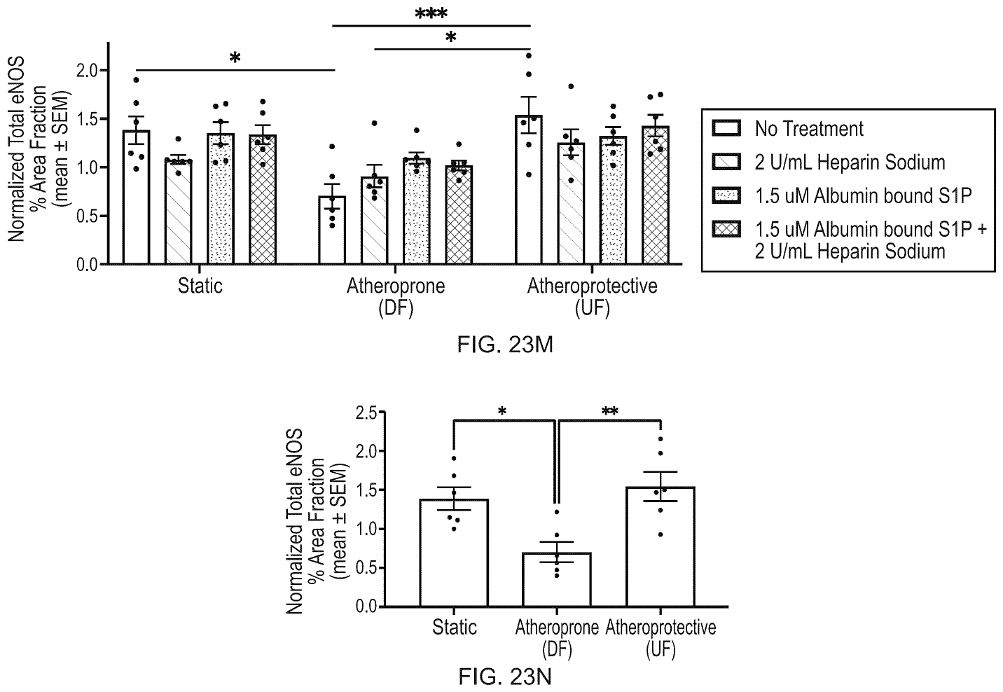

Co-Treatment improves vascular tone via upregulation of p-eNOS and reduction in vessel wall thickness: Vascular tone was studied in vitro by investigating p-eNOS and eNOS. FIG. 14 shows representative images of the percent area fraction of p-eNOS (active eNOS; green) in HCAECs subject to different conditions for 12 hours, while FIG. 15 shows representative images of total eNOS. The data was normalized to 0-hour static baseline conditions. In untreated conditions, normalized p-eNOS expression in static HCAECs was 1.53±0.09, DF HCAECs was 1.00±0.10, and UF HCAECs was 2.29±0.28. Total eNOS expression in static HCAECs was 1.38±0.36, 0.71±0.31 for DF HCAECs, and was 1.54±0.46 for UF HCAECs. These p-eNOS and eNOS trends are expected based on the well-understood relative effects of various flow conditions on p-eNOS and eNOS [11,16,56,57]. Upon exposure to co-treatment with albumin-S1P and heparin, p-eNOS expression by static HCAECs rose to 1.69±0.31, DF HCAECs rose to 2.18±0.45, and UF HCAECs remained nearly consistent at 2.00±0.16 (FIG. 14). The impact of the co-treatment on p-eNOS is the most statistically significant in DF conditions (FIG. 14). No statistical significance was observed for total eNOS in HCAECs in any flow condition (static, DF, or UF) when comparing untreated conditions to co-treatment conditions (FIG. 15). This signifies that the albumin-S1P and heparin co-treatment primarily exerts its impact on p-eNOS. FIGS. 16 and 23 contain results that show p-eNOS and total eNOS expression in HCAECs that were treated with albumin-bound S1P or heparin separately.

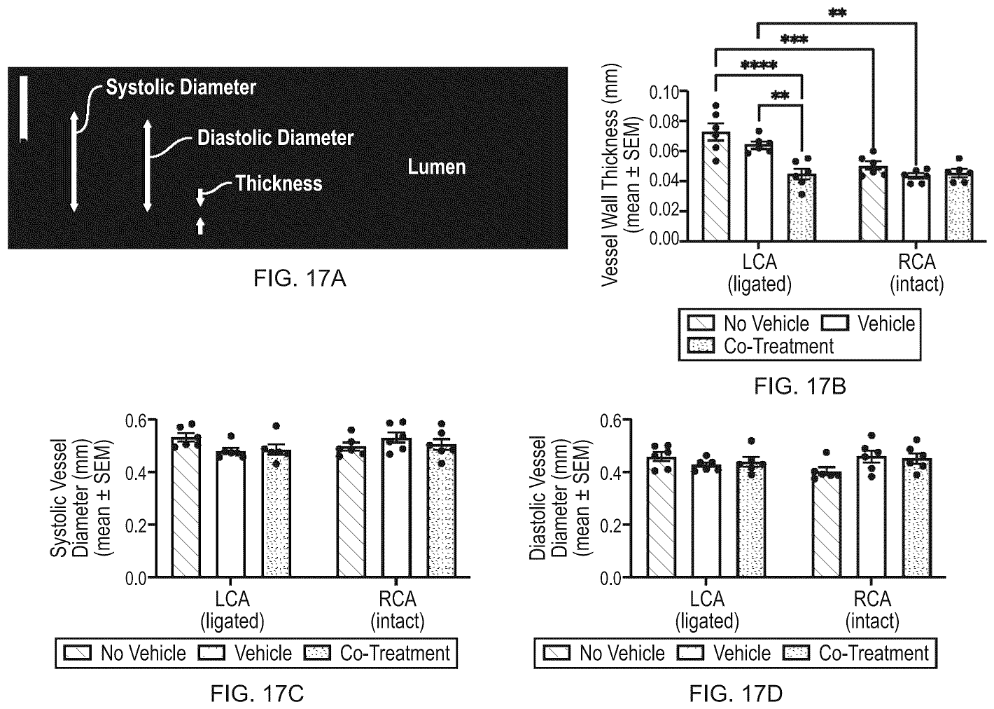

To study vascular tone in vivo, vessel remodeling was investigated by periodically observing vessel wall thickness (mm) and systolic and diastolic vessel diameters (mm) via ultrasound. FIG. 17 presents data collected five days post-ligation of the left carotid artery (LCA) of mice, with the right carotid artery (RCA) left intact to serve as a reference. The untreated LCA wall thickness was 0.07±0.01 mm, while the untreated RCA wall thickness was 0.05±0.003 mm (FIG. 17B). For the vehicle-only group, the LCA wall was 0.06±0.002 mm thick compared to 0.04±0.002 mm for the RCA. In the albumin-S1P and heparin co-treated group, the LCA wall measured 0.05±0.004 mm, with the RCA at 0.05±0.002 mm. These results indicate that the LCA wall is significantly thicker than the RCA wall in both untreated and vehicle-only conditions, but co-treatment renders LCA thickness statistically similar to RCA thickness. FIGS. 17C and 10D show the systolic and diastolic vessel diameters in mice across various experimental cohorts. The systolic vessel diameter of the ligated LCA was 0.53±0.02 mm in untreated mice, 0.48±0.01 mm in vehicle-only mice, and 0.48±0.01 mm in co-treated mice (FIG. 17C). The diastolic vessel diameter was 0.46±0.02 mm for untreated mice, 0.42±0.01 mm for vehicle-only mice, and 0.43±0.02 mm for co-treated mice (FIG. 17D). In the RCA, the systolic vessel diameter was 0.49±0.02 mm in untreated conditions, 0.53±0.02 mm in vehicle-only conditions, and 0.50±0.02 mm in co-treated conditions (FIG. 17C). The RCA diastolic vessel diameter was 0.40±0.02 mm in untreated conditions, 0.46±0.02 mm in vehicle-only conditions, and 0.45±0.02 mm in co-treated conditions (FIG. 17D). No statistically significant differences were observed across all experimental conditions for systolic and diastolic diameters. Since the systolic and diastolic diameters of the LCA and RCA remained similar across all experimental conditions, no discernible impact of co-treatment on vessel diameters was observed. This indicates that the co-treatments impact on vascular tone is most effectively exerted by normalizing vascular wall thickness.

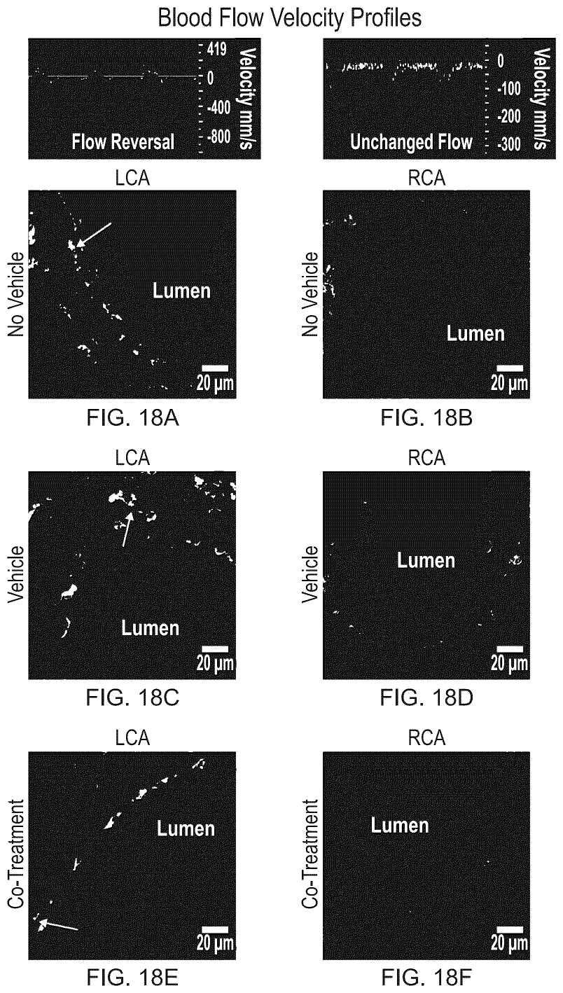

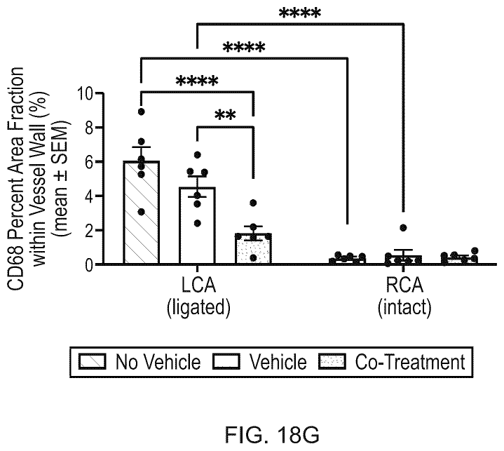

Co-Treatment reduces inflammation and vessel hyperpermeability in atheroprone region in vivo: FIG. 18 shows 40× representative images of positive-CD68 immunofluorescence, which identified macrophages taken up into the vessel walls of the carotid arteries in all cohorts of mice. A percent area fraction was calculated to determine macrophage infiltration within vessel walls of carotid arteries. Macrophage uptake in the ligated LCA of untreated (no vehicle) mice was 6.05±0.80%, vehicle-only mice was 4.52±0.60%, and mice administered the co-treatment was 1.81±0.43%. These outcomes were found to be statistically significantly different. In contrast to the ligated LCA vessels, there was far less macrophage uptake in the non-ligated RCA vessels. RCA vessels were infiltrated by macrophages at 0.35±0.07% in untreated (no vehicle) conditions, 0.53±0.32% in vehicle-only conditions, and 0.40±0.10% in co-treatment conditions. No statistical differences were observed across all cohorts when observing macrophage infiltration of the reference RCA in mice.

### Co-Treatment Mechanism of Action:

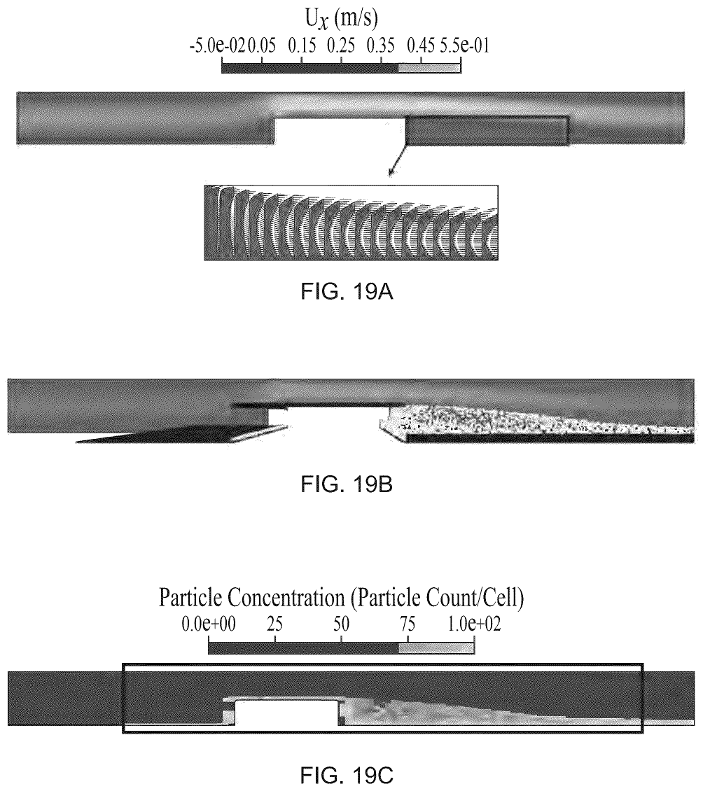

Co-treatment circulates longer and at higher concentrations in regions of DF: Computational fluid simulations confirm a region of recirculating flow downstream of the step within the custom-made in vitro parallel-plate flow chamber device (FIG. 19). To investigate the impact of the step on particle residence time, particles were uniformly released throughout the domain illustrated by the black rectangle in FIG. 19. The number of particles still suspended within the fluid at t=0.2 s after being released were counted. The particle concentration within the circulating region was notably greater than that outside of the recirculating region (FIG. 19C). The greater concentration in the recirculating region shows that particles in this region tend to reside longer than particles outside of this region, which are downstream of the computational domain illustrated. This corroborates the notion that the co-treatment has a higher concentration and longer residence time in the atheroprone DF region in vitro.

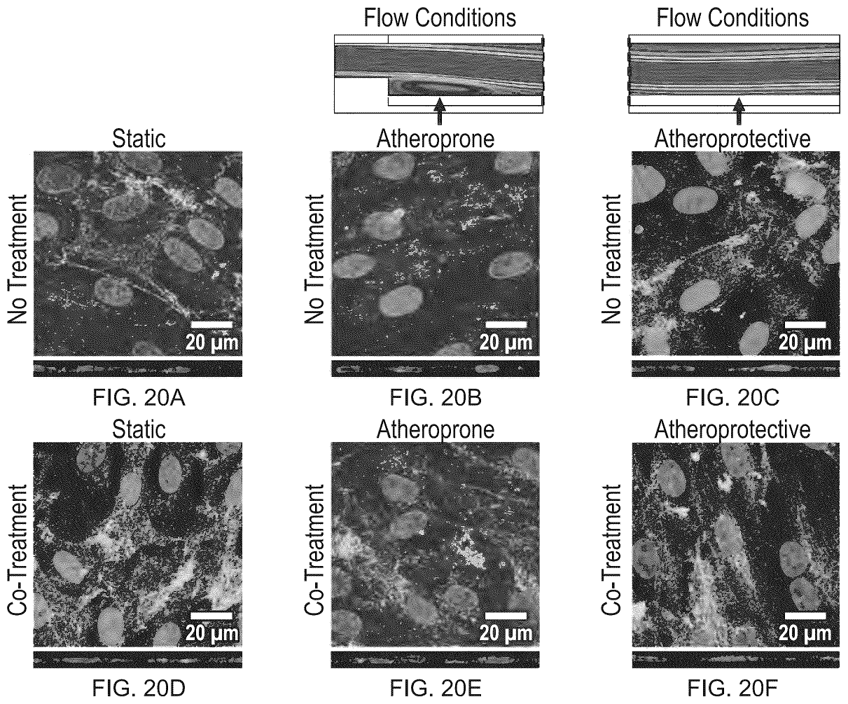

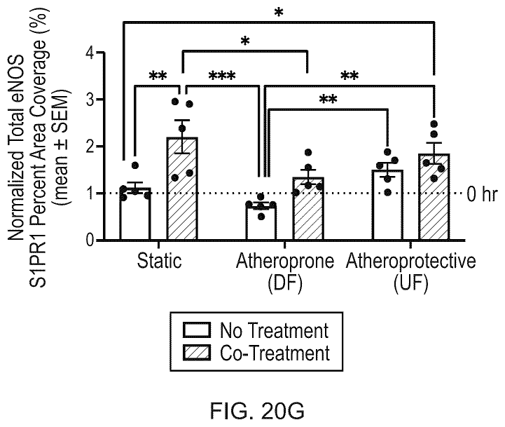

Co-treatment appears to impact SIPRI expression in regions of DF: As previously mentioned, S1PR1 is critical for EC mechanotransduction, regulation of endothelial barrier maintenance, vascular tone, and inflammation [34,58]. S1PR1-associated signaling pathways may explain how the albumin-S1P and heparin co-treatment restores endothelial barrier integrity and vascular tone in the atheroprone-DF region. FIG. 20 presents representative images of S1PR1 expression (green) in HCAECs exposed for 12 hours to different mechanical stimuli, with or without treatment. The full statistical analysis for normalized S1PR1 percent area coverage of HCAECs among the different cohorts can be found in Table 5, while key results are shown in FIG. 20G with the data points normalized with respect to baseline conditions at 0 hours. To summarize, without treatment, normalized S1PR1 expression was found to be 1.12±0.12 under static conditions, 0.74±0.07 under DF conditions, and 1.50±0.15 under UF conditions. The difference between DF and UF S1PR1 expression is statistically significant. With albumin-S1P and heparin co-treatment, compared to untreated results, HCAEC S1PR1 expression increased significantly to 2.21±0.35 under static conditions, and remained sustained with 1.85±0.22 under UF conditions. In DF conditions, co-treatment, compared to no treatment, increased HCAEC S1PR1 expression to 1.35±0.16. Although no statistical significance was present in the DF region when comparing co-treatment vs no treatment, these findings show a promising trend.

Table 5. Full statistical analysis of normalized S1PR1 percent area coverage of HCAECs among the different cohorts. Selected results from this analysis are highlighted in the plots shown in FIG. 20.

| Normalized S1PR1 | | | |
|---|---|---|---|
| Percent Area | | | |
| Coverage | Significance? | Summary | Adjusted P Value |
| Static: NoTreatment | Yes | ** | 0.0080 |
| vs. Static: Co- | | | |
| Treatment | | | |
| Static: NoTreatment | No | ns | 0.9917 |
| vs. DF: NoTreatment | | | |
| Static: NoTreatment | No | ns | >0.9999 |
| vs. DF: Co-Treatment | | | |
| Static: NoTreatment | Yes | * | 0.0403 |
| vs. UF: NoTreatment | | | |
| Static: NoTreatment | No | ns | 0.2940 |
| vs. UF: Co-Treatment | | | |
| Static: Co-Treatment | Yes | *** | 0.0007 |
| vs. DF: NoTreatment | | | |
| Static: Co-Treatment | Yes | * | 0.0254 |
| vs. DF: Co-Treatment | | | |
| Static: Co-Treatment | No | ns | 0.2286 |
| vs. UF: NoTreatment | | | |
| Static: Co-Treatment | No | ns | 0.8061 |
| vs. UF: Co-Treatment | | | |
| DF: NoTreatment vs. | No | ns | 0.8431 |
| DF: Co-Treatment | | | |
| DF: NoTreatment vs. | Yes | ** | 0.0018 |
| UF: NoTreatment | | | |
| DF: NoTreatment vs. | Yes | * | 0.0292 |
| UF: Co-Treatment | | | |
| DF: Co-Treatment | No | ns | 0.9958 |
| vs. UF: NoTreatment | | | |
| DF: Co-Treatment | No | ns | 0.6352 |
| vs. UF: Co-Treatment | | | |
| UF: NoTreatment vs. | No | ns | 0.9991 |
| UF: Co-Treatment | | | |

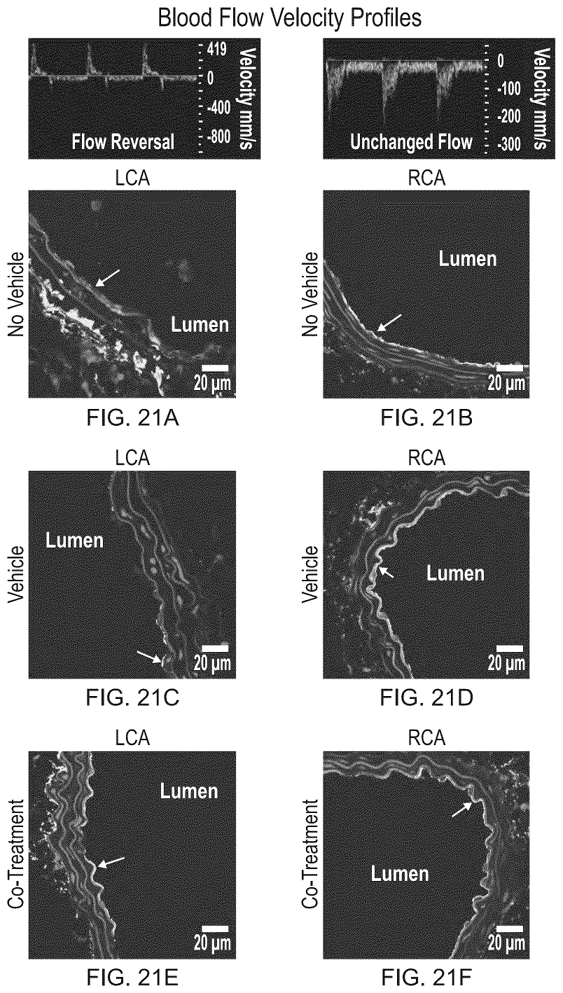

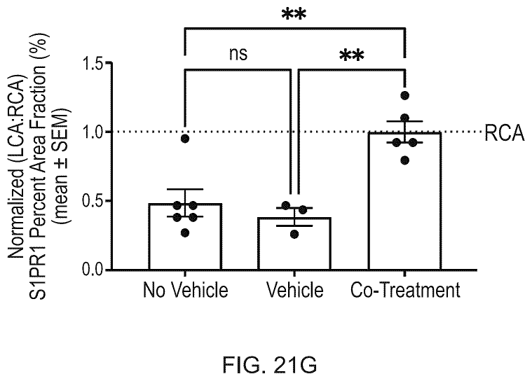

To validate these promising in vitro trends and assess statistically significant differences, in vivo studies were conducted. FIG. 21 shows 40× images of S1PR1 expression (red) in the endothelium of carotid arteries across all mouse cohorts. As usual, the RCA served as the control for each mouse, and LCA data was normalized to the RCA per condition to assess fold change in S1PR1 expression. Normalized S1PR1 expression in LCA vessels, relative to RCA, was 0.48±0.10 in untreated conditions, 0.38±0.07 in vehicle-only conditions, and 1.00±0.08 in co-treatment conditions. The increase in S1PR1 expression with co-treatment was statistically significant compared to both the vehicle and no vehicle groups. This suggests that S1PR1 expression can be positively impacted in DF regions through co-treatment by potentially activating S1PR1-associated signaling pathways to improve endothelial health.

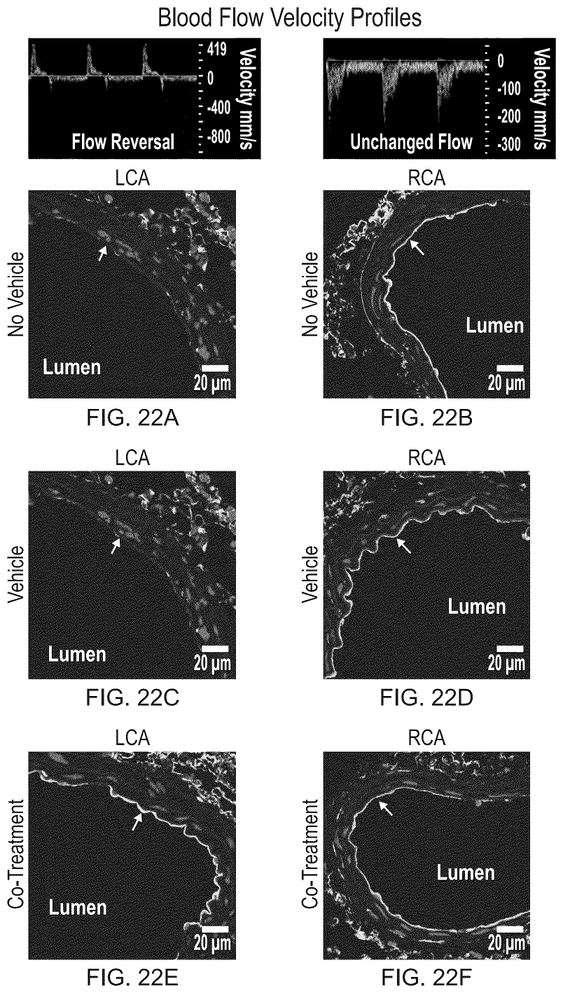

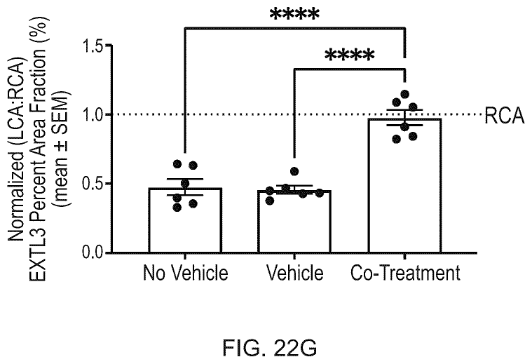

Co-treatment upregulates expression of EXTL3: EXTL3 is part of the exostosin family of genes that encodes glycosyltransferases involved in HS biosynthesis [59]. EXTL3 has dual functions: it adds N-acetylglucosamine (GlcNAc) to the protein linkage region of GAGs, which is its GlcNAc-TI activity, and to the growing HS chain, which is its GlcNAc-TII activity [59]. This makes EXTL3 an enzyme that plays a crucial role in both the initiation and elongation of HS chains [59]. Upregulation of EXTL3 after co-treatment could potentially synthesize new endothelial GCX in atheroprone DF regions. FIG. 22 shows 40× images of EXTL3 (red) in the endothelium of carotid arteries across all mouse cohorts. FIG. 22G shows the normalized percentage area fraction of EXTL3 on the carotid artery endothelium, with LCA data normalized to RCA as a control to assess fold changes in EXTL3 expression. Normalized EXTL3 expression in LCA vessels, relative to RCA, was 0.48±0.060 in untreated conditions, 0.46±0.030 in vehicle-only conditions, and 0.98±0.060 in co-treatment conditions. The increase in EXTL3 expression with co-treatment was statistically significant compared to both untreated and vehicle-only conditions.

### Discussion

This disclosure showed that treating cells with albumin-bound S1P and heparin together can restore the integrity of the GCX and improve endothelial function in regions of the vasculature prone to atherosclerosis. Evidence of GCX improvement came from WGA-labeled GCX, HS, and core proteins like SDC1 and GPC1. Restored endothelial function was marked by improvements in vascular tone and barrier functionality. While previous research has shown that S1P and heparin separately improve EC GCX integrity [36,60-63], the present disclosure is the first to combine these components and demonstrate their effects in complex and clinically relevant models. We found that the co-treatment enhances GCX integrity in primary human coronary artery ECs (HCAECs), which are highly relevant to cardiovascular disease. Additionally, in vivo application of the co-treatment in a ligated left carotid artery (LCA) mouse model under DF conditions showed significant improvements in GCX integrity. Mechanistically, the present disclosure is the first to reveal that the co-treatment upregulates EXTL3, the key enzyme involved in HS synthesis in vivo.

We believe the albumin-bound S1P and heparin co-treatment has pleiotropic effects on EC GCX health by synthesizing and protecting GCX structure. Co-treatment of S1P and heparin can protect and synthesize nascent GCX. Zeng et al. [36,62] have shown that S1P prevents SDC1 shedding via inhibition of activation of matrix metalloproteinases and synthesizes the GCX via phosphoinositide 3-kinase (PI3K) pathway, particularly in rat fat pad ECs. Additionally, heparin is thought to protect the EC GCX from degradation, particularly in sepsis, by acting as an inhibitor of heparanase, an enzyme that cleaves HS from the EC GCX [64]. This was confirmed in a preclinical study that reported GCX shedding in lung microvessels due to HS degradation induced by tumor necrosis factor-alpha (TNF-α) dependent heparanase activation [60]. This was attenuated by heparin treatment in a lipopolysaccharide murine model [60]. Furthermore, Potje et al. [63] stated that heparin can prevent the shedding of the EC GCX in human umbilical vein ECs exposed to plasma samples from COVID-19 patients, and heparin can also restore redox balance by inhibiting the activity of heparanase.

We witnessed in vitro that the albumin-bound S1P and heparin co-treatment stimulated WGA, HS, and SDC1 overexpression beyond the untreated atheroprotective UF-levels that were expected to be the threshold. Since degradation of EC GCX is detrimental to overall vascular function and health, overexpression of EC GCX could also be harmful and lead to pathological disruptions. For instance, an excessively thick EC GCX can impede blood flow and hinder requisite nutrient exchange between circulating blood and surrounding tissues [65,66]. However, no WGA, SDC1, and GPC1 overexpression was observed in the in vivo murine model, as the LCA:RCA normalized levels of percent area coverage and thickness were close to 1 in co-treatment conditions. The differing results can be attributed to the discrepancy in co-treatment exposure times: HCAECs were exposed to the co-treatment for 12 hours in dynamic conditions, while mice were administered the co-treatment for 30 minutes prior to data extraction.

The present disclosure found that co-treatment had pleiotropic effects in combating vascular dysfunction by regulating vascular tone and vessel remodeling and reducing vascular permeability. Initial experiments focused on examining EC morphology, a standard approach in the EC research field. Chronic application of UF induces alignment of EC monolayers to the direction of flow and elongation of cell shape [67], along with the planar polarity of intracellular organelles such as the microtubule and the Golgi apparatus [68]. Furthermore, Jung et al. [34] showed that shear-induced EC alignment can be significantly suppressed through S1PR1 downregulation by shRNA or FTY720-P treatment (potent functional antagonist). Jung's team also determined that the re-expression of S1PR1 restored EC alignment to the direction of flow [34]. However, in the present disclosure, albumin-S1P and heparan co-treatment did not improve EC alignment with flow (OOP metric) or among cells (MPA metric) in atheroprone-DF regions (FIG. 13). This discrepancy may be due to differences in experimental conditions, such as our use of 12-hour flow exposure on human cells versus prolonged or chronic flow in prior studies on other species.

When we examined remodeling at the tissue level via ultrasound, we found that vessel wall thickness was substantially reduced in the ligated LCA (DF) in mice administered with co-treatment. Previous studies have also shown the impact that S1P and heparin can individually have on vessel wall thickness. For instance, S1P has been shown to reduce blood pressure by activating S1PR1-mediated release of vasodilatory NO [69]. In pulmonary arterial hypertension, S1P-treated mesenchymal stem cells reduced right ventricular systolic blood pressure and caused a significant reduction in the right ventricular weight ratio and pulmonary vascular wall thickness [70]. Furthermore, heparin can influence vascular wall structure by inhibiting smooth muscle cell proliferation and migration [71-73]. Snow et al. [74] determined that two weeks of heparin treatment prevented intimal thickening and decreased the elastin content in the extracellular matrix domain in the upper and lower arterial intima after rats were subjected to LCA balloon injury. In the present disclosure, the combination of albumin-bound S1P and heparin decreased vessel wall thickness in just 30 minutes in ligated LCA, suggesting that the co-treatment is more potent than individual treatments of S1P and heparin. While there was detectable vessel wall thickening, and while it could be counteracted on by the albumin-S1P and heparin co-treatment, there were no differences observed in systolic and diastolic vessel diameters throughout all cohorts of mice. Untreated mice did not show vessel luminal narrowing in the LCA within the 5 days after ligation. This can be attributed to this disclosure observing only the early stages of endothelial dysfunction. In contrast to the study by Nam et al. [43], which observed vessel wall thickening in apolipoprotein-E deficient mice on a high-fat diet within one week of surgery, the present disclosure used wild-type C57BL/6 mice on a chow diet. This approach aimed to slow the progression of endothelial dysfunction, allowing us to better observe the effects of the albumin-S1P and heparin co-treatment on early disease conditions. Other studies have shown the impact that S1P and heparin individually have on vessel diameter. Katunaric et al. [75] determined that S1P resulted in significant vessel dilation in human arterioles (50-200 μm in luminal diameter), except when in the presence of S1PR1 inhibitors and when expression of the receptor was reduced. Furthermore, Tangphao et al. [76] witnessed increased vasodilation with heparin administration in the dorsal hand vein of human subjects. Therefore, our future studies should be implemented with an appropriate animal model to determine if the co-treatment can increase systolic and diastolic vessel diameters. With this being said, the mechanism of heparin-induced relaxation involves an increased availability of NO related to the local release of histamine [76], prompting us to examine the NO synthesis pathway.

Corroborating the in vivo vessel wall thickening data and providing clues related to vessel relaxation and constriction, we witnessed a substantial recovery in p-eNOS expression in atheroprone DF regions in both in vitro and in vivo conditions following the administration of the co-treatment. This can be due to the fact that both S1P and heparin have been individually shown to upregulate p-eNOS and endothelial production of NO. Igarashi et al. [77] provides evidence that S1P treatment in bovine aortic ECs activates Akt, a protein kinase implicated in phosphorylation of eNOS that is mediated by G protein-coupled cell surface receptors (EDG), primarily S1P receptor-1 (S1PR1) on ECs. Thus, S1P can induce eNOS phosphorylation and regulate NO production through the PI3K/Akt pathway [78]. However, S1P-dependent eNOS activation is a double-edged sword. Studies have confirmed that S1P-dependent activation of endothelial S1PR1 receptors promotes vasorelaxation responses and antagonizes vasoconstriction by activating eNOS and the production of NO, even in blood vessels where the overall response to S1P (particularly at high doses) leads to vasoconstriction [79,80]. Hence, the usage of 1 μM of albumin-bound S1P was appropriate since no vasoconstriction (no change in systolic or diastolic diameter) was observed throughout the present in vivo studies.

Additional studies have suggested that heparin increases eNOS activity in bovine ECs through a mechanism involving inhibitory guanine nucleotide regulatory protein (Gi) [81,82]. To further elucidate the mechanism of action, Li et al. [83] found that interactions between heparin and TMEM184A, a heparin receptor that interacts with and transduces stimulation from heparin in vascular cells, elicited activation of eNOS by increasing its serine 1177 phosphorylation in a calcium-dependent manner. Upchurch et al. [82] discovered that a high dosage of heparin, similar to the levels used in acute cardiovascular treatments, can reduce the production of NO in ECs through a mechanism involving a decrease in steady-state Nos 3 mRNA and eNOS protein. A similar trend was observed with S1P. Hence, optimizing the albumin-bound S1P and heparin co-treatment concentrations is imperative to ensuring proper therapeutic efficacy. One novelty in our work comes from combining the two active biomolecules and observing that the co-treatment improves p-eNOS expression in HCAECs compared to treating HCAECs with either albumin-bound S1P or heparin individually (see FIG. 16).

Interestingly, the co-treatment did not affect total eNOS expression, although an upward trend in total eNOS expression was observed after co-treatment in vitro in the atheroprone DF region. Previous studies have shown that total eNOS expression is lower in atheroprone DF regions than in atheroprotective UF regions [11,84], which is consistent with the current disclosure's findings of reduced total eNOS expression in DF compared to UF. Harding et al. [11] found that total eNOS was expressed substantially higher in the abdominal aorta, where blood flow is UF, than in the inner curvature of the distal aortic arch, where blood flow is DF. They determined that total eNOS exhibited a drastic increase in the abdominal aorta compared to the aortic arch [11]. Although total eNOS expression does not increase in the atheroprone region after co-treatment, the present disclosure found that the proportion of active eNOS is higher in the atheroprone DF region post-co-treatment (indicated by increased p-eNOS expression). We are the first to discover that this phenomenon occurs with albumin-bound S1P and heparin co-treatment.

In addition to regulating vessel remodeling via upregulation of the eNOS vascular tone agent, the co-treatment of albumin-bound S1P and heparin also improved endothelial barrier function by reducing macrophage uptake within vessel walls and inflammation in vivo in atheroprone-DF conditions. These findings can be corroborated with studies that have identified the bioactivity of S1P and heparin individually in reversing vessel hyperpermeability [85]. Lee et al. [30] determined that S1P contributes to activating tight-junction-associated protein zona occludens-1 (ZO-1), which plays an integral role in regulating barrier integrity. ZO-1 is then redistributed to the lamellipodia and cell-cell junctions via the S1PR1/Gi/Akt/Rac pathway, which enhances barrier integrity downstream. Furthermore, Li et al. [86] discovered that heparin reduced human pulmonary microvascular EC permeability induced by lipopolysaccharide and determined that the angiopoietin (Ang)/Tie2 signaling pathway represents one of the mechanisms through which heparin exerts its protective barrier function effect. Both S1P and heparin are potent anti-inflammatory agents, which is also corroborated in the present disclosure [87,88].

Without being bound to a theory, upregulation of EXTL3 encoded protein could explain how the co-treatment can synthesize new HS in atheroprone DF regions. In this disclosure, we were the first, to our knowledge, to show that in DF regions of the vasculature, downregulation of EXTL3 was present. This can cause a reduction in HS production by ECs. Marques et al. [89] established that glycoengineered gastric cancer cells with EXTL3 gene silencing fully abolished HS expression, further corroborating EXTL3-mediated promotion of HS polymerization. We believe that the heparin portion of the co-treatment acts upon this pathway, as EXTL3 is vital for initiating the synthesis of HS chains [89] and increasing GCX expression (particularly GAG HS expression) in atheroprone-DF regions of the vasculature in vivo.

The S1PR1-mediated downstream signaling pathway is another critical avenue through which the co-treatment could act. We determined that S1PR1 expression was flow-dependent, which is corroborated by other studies [34,90]. We also determined that the co-treatment in atheroprone-DF regions in vitro and in vivo can increase S1PR1 expression, which is necessary for both acute and chronic signaling events induced by laminar shear stress in ECs. The difference in significance between in vitro and in vivo models could be due to the greater systemic relevance of the mice. Furthermore, the phosphorylation state of S1PR1 plays a pivotal role in regulating its function. Phosphorylation at serine residues supports receptor recycling to the cell surface, preserving endothelial barrier integrity [91]. On the other hand, tyrosine phosphorylation can lead to receptor behavior that disrupts barrier function, as the receptor may remain at the endoplasmic reticulum [91].

Although certain biological pathways can be attributed to the potency of the co-treatment's ability to protect and synthesize new GCX and improve complex endothelial functions, the mechanical properties of the co-treatment can also play an important role. We found via fluid and particle simulations in vitro that longer residence times and higher therapeutic concentrations are present in the atheroprone DF region, where endothelial GCX degradation and dysfunction are most prevalent [8,16,92]. Future studies should look at developing a selective targeting carrier to deliver the co-treatment (i.e. nanoparticle) to further improve its potency and efficacy in atheroprone-DF regions.

In conclusion, the endothelial GCX is a complex, protein-polysaccharide layer that assists in various endothelial functions including regulation of the barrier at the interface between the blood circulation and the tissue of the blood vessel wall and overall vascular tone health. This structure is prone to mechanical degradation in DF regions. We determined that the co-treatment of albumin-bound S1P and heparin is a potent therapeutic that can protect and synthesize new endothelial GCX. The co-treatment was also able to improve vessel hyperpermeability and vascular tone via increase in eNOS expression and vessel remodeling. This finding could translate to therapy for preventing atherosclerosis and motivate future development of new therapies targeted at the GCX to treat the early stages of vascular disease.

### REFERENCES

[1]P. Libby, J. E. Buring, L. Badimon, G. K. Hansson, J. Deanfield, M. S. Bittencourt, L. Tokgozoglu, E. F. Lewis, Atherosclerosis, Nature Reviews Disease Primers. 5 (2019) 56.
[2]W. C. R. C. W. Group, World Health Organization cardiovascular disease risk charts: revised models to estimate risk in 21 global regions, Lancet Glob Health. 7 (2019) e1332-e1345.
[3]R. Mitra, A. Nersesyan, K. Pentland, M. M. Melin, R. M. Levy, E. E. Ebong, Diosmin and its glycocalyx restorative and anti-inflammatory effects on injured blood vessels, FASEB J. 36 (2022) e22630.
[4]M. J. Cheng, R. Kumar, S. Sridhar, T. J. Webster, E. E. Ebong, Endothelial glycocalyx conditions influence nanoparticle uptake for passive targeting, Int J Nanomedicine. 11 (2016) 3305-3315.
[5]R. Mitra, J. Qiao, S. Madhavan, G. L. O'Neil, B. Ritchie, P. Kulkarni, S. Sridhar, A. L. van de Ven, E. M. C. Kemmerling, C. Ferris, J. A. Hamilton, E. E. Ebong, The comparative effects of high fat diet or disturbed blood flow on glycocalyx integrity and vascular inflammation, Transl Med Commun. 3 (2018).
[6]B. G. Schwartz, C. Economides, G. S. Mayeda, S. Burstein, R. A. Kloner, The endothelial cell in health and disease: its function, dysfunction, measurement and therapy, Int J Impot Res. 22 (2010) 77-90.
[7]A. H. Henderson, St Cyres lecture. Endothelium in control, Br Heart J. 65 (1991) 116-125.
[8]L. M. Cancel, E. E. Ebong, S. Mensah, C. Hirschberg, J. M. Tarbell, Endothelial glycocalyx, apoptosis and inflammation in an atherosclerotic mouse model, Atherosclerosis. 252 (2016) 136-146.
[9]E. E. Ebong, F. P. Macaluso, D. C. Spray, J. M. Tarbell, Imaging the endothelial glycocalyx in vitro by rapid freezing/freeze substitution transmission electron microscopy, Arterioscler Thromb Vasc Biol. 31 (2011) 1908-1915.
[10]J. M. Tarbell, L. M. Cancel, The glycocalyx and its significance in human medicine, J Intern Med. 280 (2016) 97-113.
[11]I. C. Harding, R. Mitra, S. A. Mensah, I. M. Herman, E. E. Ebong, Pro-atherosclerotic disturbed flow disrupts caveolin-1 expression, localization, and function via glycocalyx degradation, J Transl Med. 16 (2018) 364.
[12]S. Banerjee, J. G. Mwangi, T. K. Stanley, R. Mitra, E. E. Ebong, Regeneration and Assessment of the Endothelial Glycocalyx To Address Cardiovascular Disease, Industrial & Engineering Chemistry Research. 60 (2021) 17328-17347.
[13]J. M. Tarbell, M. Y. Pahakis, Mechanotransduction and the glycocalyx, J Intern Med. 259 (2006) 339-350.
[14]T. Li, X. Liu, Z. Zhao, L. Ni, C. Liu, Sulodexide recovers endothelial function through reconstructing glycocalyx in the balloon-injury rat carotid artery model, Oncotarget. 8 (2017) 91350-91361.
[15]D. Chappell, K. Hofmann-Kiefer, M. Jacob, M. Rehm, J. Briegel, U. Welsch, P. Conzen, B. F. Becker, TNF-alpha induced shedding of the endothelial glycocalyx is prevented by hydrocortisone and antithrombin, Basic Res Cardiol. 104 (2009) 78-89.
[16]S. A. Mensah, A. A. Nersesyan, I. C. Harding, C. I. Lee, X. Tan, S. Banerjee, M. Niedre, V. P. Torchilin, E. E. Ebong, Flow-regulated endothelial glycocalyx determines metastatic cancer cell activity, FASEB J. 34 (2020) 6166-6184.
[17]R. Mitra, G. L. O'Neil, I. C. Harding, M. J. Cheng, S. A. Mensah, E. E. Ebong, Glycocalyx in Atherosclerosis-Relevant Endothelium Function and as a Therapeutic Target, Curr Atheroscler Rep. 19 (2017) 63.
[18]R. N. Cao, L. Tang, Z. Y. Xia, R. Xia, Endothelial glycocalyx as a potential theriapeutic target in organ injuries, Chin Med J (Engl). 132 (2019) 963-975.
[19]A. R. Pries, T. W. Secomb, P. Gaehtgens, The endothelial surface layer, Pflugers Arch. 440 (2000) 653-666.
[20]H. H. Lipowsky, The endothelial glycocalyx as a barrier to leukocyte adhesion and its mediation by extracellular proteases, Ann Biomed Eng. 40 (2012) 840-848.
[21]A. Ushiyama, H. Kataoka, T. Iijima, Glycocalyx and its involvement in clinical pathophysiologies, J Intensive Care. 4 (2016) 59.
[22]G. S. Offeddu, C. Hajal, C. R. Foley, Z. Wan, L. Ibrahim, M. F. Coughlin, R. D. Kamm, The cancer glycocalyx mediates intravascular adhesion and extravasation during metastatic dissemination, Commun Biol. 4 (2021) 255.
[23]H. Vink, A. A. Constantinescu, J. A. Spaan, Oxidized lipoproteins degrade the endothelial surface layer: implications for platelet-endothelial cell adhesion, Circulation. 101 (2000) 1500-1502.
[24]J. M. Tarbell, E. E. Ebong, The endothelial glycocalyx: a mechano-sensor and -transducer, Sci Signal. 1 (2008) pt8.
[25]B. M. Fu, J. M. Tarbell, Mechano-sensing and transduction by endothelial surface glycocalyx: composition, structure, and function, Wiley Interdiscip Rev Syst Biol Med. 5 (2013) 381-390.
[26]P. Tessari, D. Cecchet, A. Cosma, M. Vettore, A. Coracina, R. Millioni, E. Iori, L.
Puricelli, A. Avogaro, M. Vedovato, Nitric oxide synthesis is reduced in subjects with type 2 diabetes and nephropathy, Diabetes. 59 (2010) 2152-2159. [27]M. Hermann, A. Flammer, T. F. Luscher, Nitric oxide in hypertension, J Clin Hypertens (Greenwich). 8 (2006) 17-29.
[28]S. A. Mensah, M. J. Cheng, H. Homayoni, B. D. Plouffe, A. J. Coury, E. E. Ebong,
Regeneration of glycocalyx by heparan sulfate and sphingosine 1-phosphate restores inter-endothelial communication, PLoS One. 12 (2017) e0186116. [29]S. Reitsma, D. W. Slaaf, H. Vink, M. A. van Zandvoort, M. G. oude Egbrink, The endothelial glycocalyx: composition, functions, and visualization, Pflugers Arch. 454 (2007) 345-359.
[30]J. F. Lee, Q. Zeng, H. Ozaki, L. Wang, A. R. Hand, T. Hla, E. Wang, M. J. Lee, Dual roles of tight junction-associated protein, zonula occludens-1, in sphingosine 1-phosphate-mediated endothelial chemotaxis and barrier integrity, J Biol Chem. 281 (2006) 29190-29200.
[31]R. D. Veenstra, Sphingosine-1-phosphate signals the way for Cx43-mediated cardioprotection, Cardiovasc Res. 93 (2012) 8-9.
[32]L. Zhang, M. Zeng, J. Fan, J. M. Tarbell, F. R. Curry, B. M. Fu, Sphingosine-1-phosphate Maintains Normal Vascular Permeability by Preserving Endothelial Surface Glycocalyx in Intact Microvessels, Microcirculation. 23 (2016) 301-310.
[33]K. Venkataraman, Y. M. Lee, J. Michaud, S. Thangada, Y. Ai, H. L. Bonkovsky, N. S. Parikh, C. Habrukowich, T. Hla, Vascular endothelium as a contributor of plasma sphingosine 1-phosphate, Circ Res. 102 (2008) 669-676.
[34]B. Jung, H. Obinata, S. Galvani, K. Mendelson, B. S. Ding, A. Skoura, B. Kinzel, V. Brinkmann, S. Rafii, T. Evans, T. Hla, Flow-regulated endothelial S1P receptor-1 signaling sustains vascular development, Dev Cell. 23 (2012) 600-610.
[35]A. Cartier, T. Leigh, C. H. Liu, T. Hla, Endothelial sphingosine 1-phosphate receptors promote vascular normalization and antitumor therapy, Proc Natl Acad Sci USA. 117 (2020) 3157-3166.
[36]Y. Zeng, X. H. Liu, J. Tarbell, B. Fu, Sphingosine 1-phosphate induced synthesis of glycocalyx on endothelial cells, Exp Cell Res. 339 (2015) 90-95.
[37]R. Yang, M. Chen, J. Zheng, X. Li, X. Zhang, The Role of Heparin and Glycocalyx in Blood-Brain Barrier Dysfunction, Front Immunol. 12 (2021) 754141.
[38]E. I. Oduah, R. J. Linhardt, S. T. Sharfstein, Heparin: Past, Present, and Future, Pharmaceuticals (Basel). 9 (2016).
[39]C. J. Gimblet, J. W. Ernst, K. D. Bos, A. K. Stroud, A. J. Donato, D. I. Jalal, G. L. Pierce, Effect of acute heparin administration on glycocalyx thickness and endothelial function in healthy younger adults, J Appl Physiol (1985). 136 (2024) 330-336.
[40]H. H. Lipowsky, A. Lescanic, Inhibition of inflammation induced shedding of the endothelial glycocalyx with low molecular weight heparin, Microvasc Res. 112 (2017) 72-78.
[41]S. L. Swendeman, Y. Xiong, A. Cantalupo, H. Yuan, N. Burg, Y. Hisano, A. Cartier, C. H. Liu, E. Engelbrecht, V. Blaho, Y. Zhang, K. Yanagida, S. Galvani, H. Obinata, J. E. Salmon, T. Sanchez, A. Di Lorenzo, T. Hla, An engineered S1P chaperone attenuates hypertension and ischemic injury, Sci Signal. 10 (2017).
[42]S. Galvani, M. Sanson, V. A. Blaho, S. L. Swendeman, H. Obinata, H. Conger, B. Dahlback, M. Kono, R. L. Proia, J. D. Smith, T. Hla, HDL-bound sphingosine 1-phosphate acts as a biased agonist for the endothelial cell receptor SlPl to limit vascular inflammation, Sci Signal. 8 (2015) ra79.
[43]D. Nam, C. W. Ni, A. Rezvan, J. Suo, K. Budzyn, A. Llanos, D. Harrison, D. Giddens, H. Jo, Partial carotid ligation is a model of acutely induced disturbed flow, leading to rapid endothelial dysfunction and atherosclerosis, Am J Physiol Heart Circ Physiol. 297 (2009) H1535-1543.
[44]M. J. Cheng, R. Mitra, C. C. Okorafor, A. A. Nersesyan, I. C. Harding, N. N. Bal, R. Kumar, H. Jo, S. Sridhar, E. E. Ebong, Targeted Intravenous Nanoparticle Delivery: Role of Flow and Endothelial Glycocalyx Integrity, Ann Biomed Eng. 48 (2020) 1941-1954.
[45]D. Nam, C. W. Ni, A. Rezvan, J. Suo, K. Budzyn, A. Llanos, D. G. Harrison, D. P. Giddens, H. Jo, A model of disturbed flow-induced atherosclerosis in mouse carotid artery by partial ligation and a simple method of RNA isolation from carotid endothelium, J Vis Exp. (2010).
[46]J. W. VanTeeffelen, J. Brands, C. Jansen, J. A. Spaan, H. Vink, Heparin impairs glycocalyx barrier properties and attenuates shear dependent vasodilation in mice, Hypertension. 50 (2007) 261-267.
[47]J. Fisher, F. Kahn, E. Wiebe, P. Gustafsson, T. Kander, L. Mellhammar, P. Bentzer, A. Linder, The Dynamics of Circulating Heparin-Binding Protein: Implications for Its Use as a Biomarker, J Innate Immun. 14 (2022) 447-460.
[48]A. Allen, A. Neuberger, N. Sharon, The purification, composition and specificity of wheat-germ agglutinin, Biochemical Journal. 131 (1973) 155-162.
[49]J. Jin, F. Fang, W. Gao, H. Chen, J. Wen, X. Wen, J. Chen, The Structure and Function of the Glycocalyx and Its Connection With Blood-Brain Barrier, Front Cell Neurosci. 15 (2021) 739699.
[50]E. Michalaki, V. N. Surya, G. G. Fuller, A. R. Dunn, Perpendicular alignment of lymphatic endothelial cells in response to spatial gradients in wall shear stress, Commun Biol. 3 (2020) 57.
[51]L. F. L. Wilson, T. Dendooven, S. W. Hardwick, A. Echevarria-Poza, T. Tryfona, K. Krogh, D. Y. Chirgadze, B. F. Luisi, D. T. Logan, K. Mani, P. Dupree, The structure of EXTL3 helps to explain the different roles of bi-domain exostosins in heparan sulfate synthesis, Nat Commun. 13 (2022) 3314.
[52]D. Pretorius, R. P. Richter, T. Anand, J. C. Cardenas, J. R. Richter, Alterations in heparan sulfate proteoglycan synthesis and sulfation and the impact on vascular endothelial function, Matrix Biol Plus. 16 (2022) 100121.
[53]Y. Zeng, M. Waters, A. Andrews, P. Honarmandi, E. E. Ebong, V. Rizzo, J. M. Tarbell, Fluid shear stress induces the clustering of heparan sulfate via mobility of glypican-1 in lipid rafts, Am J Physiol Heart Circ Physiol. 305 (2013) H811-820.
[54]Y. Zeng, M. Waters, A. Andrews, P. Honarmandi, E. Ebong, V. Rizzo, J. M. Tarbell, Fluid Shear Stress induces the Clustering of Heparan Sulfate via Mobility of Glypican-1 in Lipid Rafts, Am J Physiol Heart Circ Physiol. 305 H811-820.
[55]A. Koo, C. F. Dewey, Jr., G. Garcia-Cardena, Hemodynamic shear stress characteristic of atherosclerosis-resistant regions promotes glycocalyx formation in cultured endothelial cells, Am J Physiol Cell Physiol. 304 (2013) C137-146.
[56]S. E. Basehore, S. Bohlman, C. Weber, S. Swaminathan, Y. Zhang, C. Jang, Z. Arany, A. M. Clyne, Laminar Flow on Endothelial Cells Suppresses eNOS O-GlcNAcylation to Promote eNOS Activity, Circ Res. 129 (2021) 1054-1066.
[57]C. F. Yeh, S. H. Cheng, Y. S. Lin, T. P. Shentu, R. T. Huang, J. Zhu, Y. T. Chen, S. Kumar, M. S. Lin, H. L. Kao, P. H. Huang, E. Rosello-Sastre, F. Garcia, H. Jo, Y. Fang, K. C. Yang, Targeting mechanosensitive endothelial TXNDC5 to stabilize eNOS and reduce atherosclerosis in vivo, Sci Adv. 8 (2022) eab18096.
[58]N. Wang, J. Y. Li, B. Zeng, G. L. Chen, Sphingosine-1-Phosphate Signaling in Cardiovascular Diseases, Biomolecules. 13 (2023).
[59]M. Busse, A. Feta, J. Presto, M. Wilen, M. Gronning, L. Kjellen, M. Kusche-Gullberg, Contribution of EXT1, EXT2, and EXTL3 to heparan sulfate chain elongation, J Biol Chem. 282 (2007) 32802-32810.
[60]E. P. Schmidt, Y. Yang, W. J. Janssen, A. Gandjeva, M. J. Perez, L. Barthel, R. L. Zemans, J. C. Bowman, D. E. Koyanagi, Z. X. Yunt, L. P. Smith, S. S. Cheng, K. H. Overdier, K. R. Thompson, M. W. Geraci, I. S. Douglas, D. B. Pearse, R. M. Tuder, The pulmonary endothelial glycocalyx regulates neutrophil adhesion and lung injury during experimental sepsis, Nat Med. 18 (2012) 1217-1223.
[61]R. Uchimido, E. P. Schmidt, N. I. Shapiro, The glycocalyx: a novel diagnostic and therapeutic target in sepsis, Crit Care. 23 (2019) 16.
[62]Y. Zeng, R. H. Adamson, F. R. Curry, J. M. Tarbell, Sphingosine-1-phosphate protects endothelial glycocalyx by inhibiting syndecan-1 shedding, Am J Physiol Heart Circ Physiol. 306 (2014) H363-372.
[63]S. R. Potje, T. J. Costa, T. F. C. Fraga-Silva, R. B. Martins, M. N. Benatti, C. E. L. Almado, K. S. G. de Sa, V. L. D. Bonato, E. Arruda, P. Louzada-Junior, R. D. R. Oliveira, D. S. Zamboni, C. Becari, M. Auxiliadora-Martins, R. C. Tostes, Heparin prevents in vitro glycocalyx shedding induced by plasma from COVID-19 patients, Life Sci. 276 (2021) 119376.
[64]J. Hogwood, E. Gray, B. Mulloy, Heparin, Heparan Sulphate and Sepsis: Potential New Options for Treatment, Pharmaceuticals (Basel). 16 (2023).
[65]H. Hara, T. Takahashi, S. Serada, M. Fujimoto, T. Ohkawara, R. Nakatsuka, E. Harada, T. Nishigaki, Y. Takahashi, S. Nojima, Y. Miyazaki, T. Makino, Y. Kurokawa, M. Yamasaki, H. Miyata, K. Nakajima, S. Takiguchi, E. Morii, M. Mori, Y. Doki, T. Naka, Overexpression of glypican-1 implicates poor prognosis and their chemoresistance in oesophageal squamous cell carcinoma, Br J Cancer. 115 (2016) 66-75.
[66]S. Weinbaum, L. M. Cancel, B. M. Fu, J. M. Tarbell, The Glycocalyx and Its Role in Vascular Physiology and Vascular Related Diseases, Cardiovasc Eng Technol. 12 (2021) 37-71.
[67]S. Noria, D. B. Cowan, A. I. Gotlieb, B. L. Langille, Transient and steady-state effects of shear stress on endothelial cell adherens junctions, Circ Res. 85 (1999) 504-514.
[68]E. Tzima, W. B. Kiosses, M. A. del Pozo, M. A. Schwartz, Localized cdc42 activation, detected using a novel assay, mediates microtubule organizing center positioning in endothelial cells in response to fluid shear stress, J Biol Chem. 278 (2003) 31020-31023.
[69]J. Ouyang, Z. Shu, S. Chen, H. Xiang, H. Lu, The role of sphingosine 1-phosphate and its receptors in cardiovascular diseases, J Cell Mol Med. 24 (2020) 10290-10301.
[70]H. Kang, K. H. Kim, J. Lim, Y. S. Kim, J. Heo, J. Choi, J. Jeong, Y. Kim, S. W. Kim, Y. M. Oh, M. S. Choo, J. Son, S. J. Kim, H. J. Yoo, W. Oh, S. J. Choi, S. W. Lee, D. M. Shin, The Therapeutic Effects of Human Mesenchymal Stem Cells Primed with Sphingosine-1 Phosphate on Pulmonary Artery Hypertension, Stem Cells Dev. 24 (2015) 1658-1671.
[71]A. W. Clowes, M. J. Karnowsky, Suppression by heparin of smooth muscle cell proliferation in injured arteries, Nature. 265 (1977) 625-626.
[72]J. R. Guyton, R. D. Rosenberg, A. W. Clowes, M. J. Karnovsky, Inhibition of rat arterial smooth muscle cell proliferation by heparin. In vivo studies with anticoagulant and nonanticoagulant heparin, Circ Res. 46 (1980) 625-634.
[73]A. W. Clowes, M. M. Clowes, Kinetics of cellular proliferation after arterial injury. II. Inhibition of smooth muscle growth by heparin, Lab Invest. 52 (1985) 611-616.
[74]A. D. Snow, R. P. Bolender, T. N. Wight, A. W. Clowes, Heparin modulates the composition of the extracellular matrix domain surrounding arterial smooth muscle cells, Am J Pathol. 137 (1990) 313-330.
[75]B. Katunaric, G. SenthilKumar, M. E. Schulz, N. De Oliveira, J. K. Freed, S1P (Sphingosine-1-Phosphate)-Induced Vasodilation in Human Resistance Arterioles During Health and Disease, Hypertension. 79 (2022) 2250-2261.
[76]O. Tangphao, S. Chalon, H. J. Moreno, Jr., A. K. Abiose, T. F. Blaschke, B. B. Hoffman, Heparin-induced vasodilation in human hand veins, Clin Pharmacol Ther. 66 (1999) 232-238.
[77]J. Igarashi, T. Michel, Sphingosine 1-phosphate and isoform-specific activation of phosphoinositide 3-kinase beta. Evidence for divergence and convergence of receptor-regulated endothelial nitric-oxide synthase signaling pathways, J Biol Chem. 276 (2001) 36281-36288.
[78]Y. Rikitake, K. Hirata, S. Kawashima, M. Ozaki, T. Takahashi, W. Ogawa, N. Inoue, M. Yokoyama, Involvement of endothelial nitric oxide in sphingosine-1-phosphate-induced angiogenesis, Arterioscler Thromb Vasc Biol. 22 (2002) 108-114.
[79]J. Igarashi, T. Michel, S1P and eNOS regulation, Biochim Biophys Acta. 1781 (2008) 489-495.
[80]D. Kerage, R. B. Gombos, S. Wang, M. Brown, D. G. Hemmings, Sphingosine 1-phosphate-induced nitric oxide production simultaneously controls endothelial barrier function and vascular tone in resistance arteries, Vascul Pharmacol. 140 (2021) 106874.
[81]P. C. Kouretas, R. L. Hannan, N. K. Kapur, R. Hendrickson, E. M. Redmond, A. K. Myers, Y. D. Kim, P. A. Cahill, J. V. Sitzmann, Non-anticoagulant heparin increases endothelial nitric oxide synthase activity: role of inhibitory guanine nucleotide proteins, J Mol Cell Cardiol. 30 (1998) 2669-2682.
[82]G. R. Upchurch, Jr., G. N. Welch, J. E. Freedman, A. J. Fabian, A. Pigazzi, A. M. Scribner, C. S. Alpert, J. F. Keaney, Jr., J. Loscalzo, High-dose heparin decreases nitric oxide production by cultured bovine endothelial cells, Circulation. 95 (1997) 2115-2121.
[83]Y. Li, L. M. Talotta-Altenburg, K. A. Silimperi, G. O. Ciabattoni, L. J. Lowe-Krentz, Endothelial nitric oxide synthase activation is required for heparin receptor effects on vascular smooth muscle cells, Am J Physiol Cell Physiol. 318 (2020) C463-C475.
[84]Z. Wang, Q. Lv, H. Liu, Y. Wu, Y. Bai, Y. Cheng, Y. Su, Y. Cai, J. Yu, J. Ma, J. Bao, Caveolae Depletion Contributes to Vasorelaxant Effects of Chenodeoxycholic Acid, Cell Physiol Biochem. 42 (2017) 1013-1024.
[85]L. Wang, S. M. Dudek, Regulation of vascular permeability by sphingosine 1-phosphate, Microvasc Res. 77 (2009) 39-45.
[86]X. Li, Z. Zheng, Y. Mao, X. Ma, Unfractionated heparin promotes LPS-induced endothelial barrier dysfunction: a preliminary study on the roles of angiopoietin/Tie2 axis, Thromb Res. 129 (2012) e223-228.
[87]J. Fettel, B. Kuhn, N. A. Guillen, D. Surun, M. Peters, R. Bauer, C. Angioni, G. Geisslinger, F. Schnutgen, D. Meyer Zu Heringdorf, O. Werz, P. Meybohm, K. Zacharowski, D. Steinhilber, J. Roos, T. J. Maier, Sphingosine-1-phosphate (S1P) induces potent anti-inflammatory effects in vitro and in vivo by S1P receptor 4-mediated suppression of 5-lipoxygenase activity, FASEB J. 33 (2019) 1711-1726.
[88]E. Young, The anti-inflammatory effects of heparin and related compounds, Thromb Res. 122 (2008) 743-752.
[89]C. Marques, J. Pocas, C. Gomes, I. Faria-Ramos, C. A. Reis, R. R. Vives, A. Magalhaes, Glycosyltransferases EXTL2 and EXTL3 cellular balance dictates heparan sulfate biosynthesis and shapes gastric cancer cell motility and invasion, J Biol Chem. 298 (2022) 102546.
[90]A. Cantalupo, A. Gargiulo, E. Dautaj, C. Liu, Y. Zhang, T. Hla, A. Di Lorenzo, S1PR1 (Sphingosine-1-Phosphate Receptor 1) Signaling Regulates Blood Flow and Pressure, Hypertension. 70 (2017) 426-434.
[91]M. Anwar, D. Mehta, Post-translational modifications of S1PR1 and endothelial barrier regulation, Biochimica et Biophysica Acta (BBA)—Molecular and Cell Biology of Lipids. 1865 (2020) 158760.
[92]A. Milusev, R. Rieben, N. Sorvillo, The Endothelial Glycocalyx: A Possible Therapeutic Target in Cardiovascular Disorders, Front Cardiovasc Med. 9 (2022) 897087.
[93] Sigma-Aldrich. https_www_sigmaaldrich_com/US/en/product/sial/h3393.
[94] Galvani S et al. HDL-bound sphingosine 1-phosphate acts as a biased agonist for the endothelial cell receptor SIPl to limit vascular inflammation. Sci Signal. 2015 Aug. 11; 8(389):ra79. Doi: 10.1126/scisignal.aaa2581.
[95]U.S. Patent Application Publication No. US 2020/0023001 A1.
[96] International Application No. PCT/US2017/046916.
[97] Mitra, R., Pentland, K., Kolev, S., Eden, M., Levine, E., Oakes, J. M. and Ebong, E. E., 2025. Co-therapy with S1P and heparan sulfate derivatives to restore endothelial glycocalyx and combat pro-atherosclerotic endothelial dysfunction. Life Sciences, p. 123662.

### Embodiments

1. A composition comprising: a complex, wherein the complex comprises sphingosine-1-phosphate, a derivative thereof, or a pharmaceutically acceptable salt thereof, bound to a protein; and heparin, a derivative thereof, or a pharmaceutically acceptable salt thereof.

2. The composition of Embodiment 1, wherein the protein is apolipoprotein M or albumin.

3. The composition of Embodiment 1 or 2, wherein the protein is apolipoprotein M.

4. The composition of Embodiment 1 or 2, wherein the protein is albumin.

5. The composition of any one of Embodiments 1-4, wherein the derivative thereof is heparin sodium.

6. The composition of any one of Embodiments 1-5, wherein the complex has a concentration of about 0.1 μM to about 10 μM.

7. The composition of any one of Embodiments 1-6, wherein the complex has a concentration of about 1.5 μM.

8. The composition of any one of Embodiments 1-7, wherein the heparin, a derivative thereof, or a pharmaceutically acceptable salt thereof, has a concentration of about 0.1 U/mL to about 10 U/mL.

9. The composition of any one of Embodiments 1-8, wherein the heparin, a derivative thereof, or a pharmaceutically acceptable salt thereof, has a concentration of about 2 U/mL.

10. The composition of any one of Embodiments 1-9, wherein the composition comprises: the complex; and heparin or a pharmaceutically acceptable salt of heparin.

11. The composition of Embodiment 10, wherein the pharmaceutically acceptable salt of heparin is heparin sodium.

12. A conjugate, comprising sphingosine-1-phosphate, a derivative thereof, or a pharmaceutically acceptable salt thereof, conjugated to heparin, a derivative thereof, or a pharmaceutically acceptable salt thereof, through a linker.

13. The conjugate of Embodiment 12, wherein the linker is a triazole linker.

14. The conjugate of Embodiment 12 or 13, wherein the conjugate is

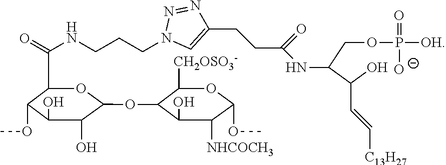

15. A method of regenerating glycocalyx of a cell, comprising contacting the cell with: i) a complex, wherein the complex comprises sphingosine-1-phosphate, a derivative thereof, or a pharmaceutically acceptable salt thereof, bound to a protein; and ii) heparin, a derivative thereof, or a pharmaceutically acceptable salt thereof, or the conjugate of any one of Embodiments 12-14.

16. A method of increasing expression of a connexin by a cell, comprising contacting the cell with: i) a complex, wherein the complex comprises sphingosine-1-phosphate, a derivative thereof, or a pharmaceutically acceptable salt thereof, bound to a protein; and ii) heparin, a derivative thereof, or a pharmaceutically acceptable salt thereof, or the conjugate of any one of Embodiments 12-14.

17. A method of increasing stability of the plasma membrane of a cell, comprising contacting the cell with: i) a complex, wherein the complex comprises sphingosine-1-phosphate, a derivative thereof, or a pharmaceutically acceptable salt thereof, bound to a protein; and ii) heparin, a derivative thereof, or a pharmaceutically acceptable salt thereof, or the conjugate of any one of Embodiments 12-14.

18. A method of improving gap junction function of a cell, comprising contacting the cell with: i) a complex, wherein the complex comprises sphingosine-1-phosphate, a derivative thereof, or a pharmaceutically acceptable salt thereof, bound to a protein; and ii) heparin, a derivative thereof, or a pharmaceutically acceptable salt thereof, or the conjugate of any one of Embodiments 12-14.

19. A method of increasing intercellular communication between two or more cells, comprising contacting at least one of the two or more cells with an effective amount of: i) a complex, wherein the complex comprises sphingosine-1-phosphate, a derivative thereof, or a pharmaceutically acceptable salt thereof, bound to a protein; and ii) heparin, a derivative thereof, or a pharmaceutically acceptable salt thereof, or the conjugate of any one of Embodiments 12-14.

20. A method of inhibiting glycocalyx degradation of a cell, comprising contacting the cell with: i) a complex, wherein the complex comprises sphingosine-1-phosphate, a derivative thereof, or a pharmaceutically acceptable salt thereof, bound to a protein; and ii) heparin, a derivative thereof, or a pharmaceutically acceptable salt thereof, or the conjugate of any one of Embodiments 12-14.

21. The method of any one of Embodiments 15-20, wherein the cell is an endothelial cell.

22. A method of treating a vascular disease in a subject in need thereof, comprising administering to the subject a therapeutically effective amount of: i) a complex, wherein the complex comprises sphingosine-1-phosphate, a derivative thereof, or a pharmaceutically acceptable salt thereof, bound to a protein; and ii) heparin, a derivative thereof, or a pharmaceutically acceptable salt thereof, or the conjugate of any one of Embodiments 12-14.

23. The method of Embodiment 22, wherein the vascular disease is atherosclerosis, a blood clot, a stroke, peripheral artery disease, an aneurysm, a pulmonary embolism, carotid artery disease, arteriovenous malformation, critical limb ischemia, deep vein thrombosis, chronic venous insufficiency, a varicose vein, coronary artery disease, Raynaud's disease or vasculitis.

24. The method of Embodiment 23, wherein the vascular disease is atherosclerosis.

25. The method of any one of Embodiments 15-24, wherein the heparin, the derivative thereof, or the pharmaceutically acceptable salt thereof, is derived from an exogenous source.

26. The method of Embodiment 25, wherein the derivative of heparin is heparan sulfate.

27. The method of Embodiment 26, wherein the pharmaceutically acceptable salt of heparin is heparin sodium.

28. The method of Embodiment 25, wherein the exogenous source is porcine intestinal mucosa.

29. A kit, comprising: a complex, wherein the complex comprises sphingosine-1-phosphate, a derivative thereof, or a pharmaceutically acceptable salt thereof, bound to a protein; and heparin, a derivative thereof, or a pharmaceutically acceptable salt thereof, or the conjugate of any one of Embodiments 12-14.

30. The kit of Embodiment 29, wherein the kit comprises: the complex; and heparin or a pharmaceutically acceptable salt of heparin.

31. The kit of Embodiment 30, wherein the pharmaceutically acceptable salt of heparin is heparin sodium.

32. A method of restoring GCX thickness and/or coverage, elevating vasodilator eNOS (endothelial-type nitric oxide synthase) expression, improving vascular tone, reducing inflammation and/or vessel hyperpermeability, and/or inhibiting vessel wall thickening of a cell, said method comprising contacting the cell with: i) a complex, wherein the complex comprises sphingosine-1-phosphate, a derivative thereof, or a pharmaceutically acceptable salt thereof, bound to a protein; and ii) heparin, a derivative thereof, or a pharmaceutically acceptable salt thereof, or with the conjugate of any one of Embodiments 12-14.

33. A method of making a composition of Embodiment 1.

34. A method of improving p-ENOS expression in a human coronary artery endothelial cell (HCAEC) comprising contacting the cell with i) a complex, wherein the complex comprises sphingosine-1-phosphate, a derivative thereof, or a pharmaceutically acceptable salt thereof, bound to a protein; and ii) heparin, a derivative thereof, or a pharmaceutically acceptable salt thereof, or with the conjugate of any one of Embodiments 12-14.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments described herein.

What is claimed is:

1. A composition comprising:
a complex, wherein the complex comprises sphingosine-1-phosphate, a derivative thereof, or a pharmaceutically acceptable salt thereof, bound to a protein; and
heparin, a derivative thereof, or a pharmaceutically acceptable salt thereof.

2. The composition of claim 1, wherein the protein is apolipoprotein M or albumin.

3. The composition of claim 1, wherein the protein is apolipoprotein M.

4. The composition of claim 1, wherein the protein is albumin.

5. The composition of claim 1, wherein the pharmaceutically acceptable salt thereof is heparin sodium.

6. The composition of claim 1, wherein the complex has a concentration of about 0.1 μM to about 10 μM.

7. The composition of claim 1, wherein the complex has a concentration of about 1.5 μM.

8. The composition of claim 1, wherein the heparin, the derivative thereof, or the pharmaceutically acceptable salt thereof, is present in an amount of about 100 U/kg to about 1000 U/kg based on a subject's weight.

9. The composition of claim 1, wherein the heparin, the derivative thereof, or the pharmaceutically acceptable salt thereof, is present in an amount of about 500 U/kg.

10. A method of regenerating glycocalyx of a cell or inhibiting glycocalyx degradation of a cell, comprising contacting the cell with:
a complex, wherein the complex comprises sphingosine-1-phosphate, a derivative thereof, or a pharmaceutically acceptable salt thereof, bound to a protein; and
heparin, a derivative thereof, or a pharmaceutically acceptable salt thereof.

11. The method of claim 10, wherein the cell is a mammalian cell.

12. A method of treating a vascular disease in a subject in need thereof, comprising administering to the subject a composition comprising:
a complex, wherein the complex comprises sphingosine-1-phosphate, a derivative thereof, or a pharmaceutically acceptable salt thereof, bound to a protein; and
heparin, a derivative thereof, or a pharmaceutically acceptable salt thereof.

13. The method of claim 12, wherein the vascular disease is atherosclerosis, a blood clot, a stroke, peripheral artery disease, an aneurysm, a pulmonary embolism, carotid artery disease, arteriovenous malformation, critical limb ischemia, deep vein thrombosis, chronic venous insufficiency, a varicose vein, coronary artery disease, Raynaud's disease, sepsis, von Willebrand disease or vasculitis.

14. The method of claim 12, wherein the vascular disease is atherosclerosis, carotid artery disease, or coronary artery disease.

15. The method of claim 12, wherein the protein is apolipoprotein M or albumin.

16. The method of claim 12, wherein the complex has a concentration of about 0.1 μM to about 10 μM.

17. The method of claim 12, wherein the complex has a concentration of about 1.5 μM.

18. The method of claim 12, wherein the heparin, the derivative thereof, or the pharmaceutically acceptable salt thereof, is present in an amount of about 100 U/kg to about 1000 U/kg based on the subject's weight.

19. The method of claim 12, wherein the heparin, the derivative thereof, or the pharmaceutically acceptable salt thereof, is present in an amount of about 500 U/kg.

20. A kit, comprising the composition of claim 1.
